(19) United States
(12) Patent Application Publication
(10) Pub. No.: US 2024/0409921 A1
(43) Pub. Date: Dec. 12, 2024

(54) METHODS AND COMPOSITIONS FOR CHARACTERIZING NUCLEIC ACIDS MOLECULES IN INDIVIDUAL CELLS

(71) Applicant: University of Washington, Seattle, WA (US)

(72) Inventor: Zhi-jun Duan, Seattle, WA (US)

(73) Assignee: University of Washington, Seattle, WA (US)

(21) Appl. No.: 18/733,640

(22) Filed: Jun. 4, 2024

Related U.S. Application Data
(60) Provisional application No. 63/471,495, filed on Jun. 6, 2023.

(51) Int. Cl. C12N 15/10; B01L 3/00; C12Q 1/25; C12Q 1/6806; C12Q 1/683; C12Q 1/6869; G01N 33/543

(52) U.S. Cl. CPC C12N 15/1065; B01L 3/502761; C12N 15/1096; C12Q 1/25; C12Q 1/6806; C12Q 1/683; C12Q 1/6869; G01N 33/54326; B01L 2200/16; B01L 2300/0663; B01L 2300/18; B01L 2400/0487; G01N 2333/9015

(57) ABSTRACT

Methods and systems for characterizing the spatial genomic organization and gene expression in single cells is described. The methods described herein is used to generate spatial and transcriptomic libraries to determine the three-dimensional organization associated with the expression of nucleic acids. Implementations described herein can be used for research or diagnostic purposes.

### CROSS-REFERENCE TO RELATED APPLICATION(S)

This application claims the priority of U.S. Provisional Patent Application No. 63/471,495, filed on Jun. 6, 2023, and which is incorporated by reference herein in its entirety.

### STATEMENT REGARDING FEDERALLY SPONSORED RESEARCH OR DEVELOPMENT

This invention was made with government support under Grant No. 1R01HG012303, awarded by the National Human Genome Research Institute and Grant No. 1R61DA047010, awarded by the National Institute of Drug Abuse. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is W149-0053US_Seq.xml. The file is 2,642 bytes, was created Jun. 4, 2024, and is being submitted electronically via Patent Center.

### TECHNICAL FIELD

This application relates to methods for characterizing the spatial arrangement and sequence of nucleic acids, and more particularly, with single cell resolution.

### BACKGROUND

Mammalian genomes are highly organized in the three-dimensional (3D) nuclear space (Dekker et al. 2017), characterized by forming various architectural structures at different genomic scales, such as chromosome territories (CTs) (Cremer and Cremer 2001), large-scale active or repressed compartments (A/B compartments) (Rao et al. 2014), subcompartments (Rao et al. 2014; Xiong and Ma 2019), topologically associating domains (TADs) (Dixon et al. 2012; Nora et al. 2012) and subTADs (Phillips-Cremins et al. 2013; Beagan and Phillips-Cremins 2020), and chromatin loops (Salameh et al. 2020; Tang et al. 2015). Growing evidence has suggested that these genome structural features are intertwined with multiple layers of gene regulation and other genome functions (Oudelaar and Higgs 2021), playing crucial roles in development and disease (Marchal, Sima, and Gilbert 2019; J. Ma and Duan 2019; Zheng and Xie 2019; Misteli 2020; Spielmann, Lupiáñez, and Mundlos 2018). However, it remains poorly understood how the changes of multiscale 3D genome structure in a given single cell inform the cell's transcriptional programming and thereby impact cellular phenotypes in health and disease.

### BRIEF DESCRIPTION OF THE DRAWINGS

Many of the drawings submitted herein are better understood in color. Applicant considers the color versions of the drawings as part of the original submission and reserves the right to present color images of the drawings in later proceedings.

FIG. 1 illustrates an example environment for characterizing the spatial genomic organization and gene expression in a cell.

FIG. 2 illustrates an example process 200 for characterizing the spatial genomic organization and the gene expression in single cells.

FIG. 3 illustrates an example of a system 300 for performing various functions described herein.

FIGS. 4A-4D illustrate the molecular design of GAGE-seq adaptors and the molecular structure of the DNA fragments in GAGE-seq scHi-C and scRNA libraries.

FIGS. 5A-5E illustrate an overview and validation of GAGE-seq.

FIGS. 6A-6K illustrate high-quality scHi-C and scRNA-seq data generated by GAGE-seq.

FIGS. 7A-7F illustrate quality-control assessment of the K562-GM12878 GAGE-seq libraries.

FIGS. 8A-8F illustrate quality-control assessment of the MDS-L GAGE-seq library.

FIGS. 9A-9C illustrate single-cell and pseudo-bulk contact maps from the GAGE-seq datasets at the beta globin locus.

FIGS. 10A-10E illustrate cell cycle analysis of the GAGE-seq K562 cells.

FIG. 11 illustrates aggregated single-cell gene expression profiles of the genes in the GAPDH locus.

FIGS. 12A-12C illustrate a comparison of estimated scRNA and scHi-C library complexities between GAGEseq and HiRES28.

FIG. 13 illustrates a comparison between GAGE-seq and other scHi-C related methods in terms of efficiency.

FIGS. 14A-14D illustrate a quality-control assessment of the GAGE-seq mouse brain cortex library.

FIGS. 15A-15F illustrate a quality-control assessment of the GAGE-seq mouse brain cortex library.

FIGS. 16A-16G illustrate cell types in mouse cortex characterized by GAGE-seq scHi-C and scRNA-seq.

FIG. 17 illustrates high resolution cell type identification in the mouse brain cortex using GAGE-seq.

FIG. 18 illustrates high resolution inhibitory neuron subtypes revealed by GAGE-seq.

FIG. 19 illustrates high resolution excitatory neuron subtypes revealed by GAGE-seq.

FIG. 20 illustrates congruence between GAGE-seq scRNA-seq clusters and scHi-C embeddings.

FIGS. 21A-21D illustrate re-analysis of scRNA profiles of HiRES and downsampled GAGE-seq from the mouse brain.

FIGS. 22A-22H illustrate membership correspondence between GAGE-seq and MERFISH datasets.

FIGS. 23A-23P illustrate high correlation between cortical layer-specific gene expression and the in situ dynamics of the 3D genome features of excitatory neurons.

FIGS. 24A-24G illustrate that 3D genome features inform cell type-specific gene expressions in the mouse cortex.

FIGS. 25A-24E illustrate correlation between cell type-specific single-cell A/B value and gene expression when comparing Pvalb and the other inhibitory neurons.

FIGS. 26A-26C illustrate aggregated single-cell insulation score and scA/B value of the four gene loci, Grik2, Dscam, Rbfox1 and Nrxn3 in the annotated 28 cell subtypes.

FIG. 27 illustrates aggregated contact maps of the Dscam and Nrxn3 gene loci showing cell type-specific domain organization.

FIGS. 28A-28H illustrate the correlation between gene expression and 3D genome features at the single-cell level.

FIGS. 29A-29C illustrate the joint influence of A/B compartment and chromatin accessibility on gene expression.

FIGS. 30A-30F illustrate integrative analysis of GAGE-seq and chromatin accessibility in the mouse cortex.

FIGS. 31A-31C illustrate the refinement of gene-CRE pairs enabled by GAGE-seq.

FIGS. 32A-321 illustrate the interplay between 3D genome variation and gene expression changes in human bone marrow differentiation.

FIGS. 33A-33C illustrate the 3D genome reorganization at the gene loci of the B-NK cell differentially expressed (DE) genes with different gene lengths.

### DETAILED DESCRIPTION

Molecular and cellular heterogeneity is intrinsic to cell differentiation and tissue development. The recent advent of single-cell technologies has been transformative in overcoming cells' heterogeneous nature. For example, high-throughput single-cell RNA-seq (scRNA-seq) analyses have enabled the identification of cell subtypes at unprecedented resolution in complex tissues (Cao et al. 2020; Calderon et al. 2022). Single-cell Hi-C (scHi-C) technologies, which map chromatin interactions in individual cells (Nagano et al. 2013; Ramani et al. 2017; Nagano et al. 2017; Flyamer et al. 2017; Stevens et al. 2017; Tan et al. 2018, 2021; Li et al. 2019), have allowed the characterization of the 3D genome architecture of distinct cell types in complex tissues (Tan et al. 2021). However, to fully understand the causal dependencies between the 3D genome organization and transcriptional activities in a cell, it concurrent measurement of the two molecular properties in the same cell(s) may be required. Although computational methods are able to provide integrative analysis of scHi-C and scRNA-seq to some degree (Tan et al. 2021), (Zhang, Zhou, and Ma 2021), it was not previously possible to faithfully match a cell's 3D genome organization with its gene regulation programs based on separately generated scHi-C and scRNA-seq data. While imaging-based technologies can simultaneously visualize and measure both genome architecture and transcripts in single cells, these methods rely on highly specific equipment and are currently limited in throughput (Cardozo Gizzi et al. 2019; Mateo et al. 2019; Su et al. 2020; Takei et al. 2021). Thus, new high-throughput genomic technologies that are able to co-assay 3D genome and gene expression in the same cell are urgently needed.

It has been shown that single-cell multimodal technologies, which can jointly profile multiple molecular phenotypes/genotypes from the same cell, are able to uncover the underneath connections between the different molecular properties of the cell (Macaulay, Ponting, and Voet 2017; Zhu, Preissl, and Ren 2020; Hao et al. 2021). To interrogate the relationship between genome structure and gene regulation at the single cell level, the present disclosure describes techniques related to GAGE-seq (Genome Architecture and Gene Expression by SEQuencing), a highly scalable approach for individual or joint mapping of single-cell landscapes of chromatin interactions and gene expression at low cost. Implementations of GAGE-seq described herein provide high-throughput, single-cell co-assay methods for concurrent measurement of genome-wide 3D chromatin interactions and transcriptome in the same single cells.

This disclosure also describes experimental validation for GAGE-seq. Using GAGE-seq, four different cell lines and two tissue types, including mouse brain and human bone marrow, were profiled. High-quality GAGE-seq datasets were generated with a wide variety of mouse and human cell lines and primary tissue cells, including GM12878, K562, MDS-L, NIH3T3, mouse brain cortex and human bone marrow CD34+ cells. Both single-cell Hi-C and the scRNA-seq in GAGE-seq show high robustness, specificity, sensitivity and reproducibility. Importantly, GAGE-seq was shown to uniquely reveal genome structure-function relationship in primary tissue context, leading to intricate and dynamic connections between cell type-specific 3D genome features and cell type-specific gene expression in single cells that may inform cell fate decision-making during hematopoiesis. Combining GAGE-seq and in situ spatial transcriptome data in the mouse brain further demonstrates the potential of integrative and multi-omic delineation of complex tissues.

In some implementations, GAGE-seq can be implemented in a microfluidic platform or a microfluidic circuit. The term “microfluidic circuit,” and its equivalents, as used herein, can refer to an apparatus that channels, manipulates, or otherwise is configured to contain volumes of a fluid (e.g., sample and/or reagent) in a range from 0.1 microliters (μL) to 999 μL, such as from 1-100 μL, or from 2-25 μL. Similarly, a “microfluidic cartridge,” and its equivalents, may include various components and channels that are configured to accept, retain, or facilitate passage of microfluidic volumes of sample or reagent. Certain implementations described herein can also function with nanoliter volumes (in the range of 10-500 nanoliters (nL), such as 100 nL).

Various implementations described herein relate to techniques for generating DNA libraries that are indicative of 3D organization of chromatin and simultaneous transcriptional activity. In various cases, permeabilized cells are subjected to a protocol that generates both transcriptomic DNA ((DNA) and spatial DNA (sDNA) before the cells are fully lysed and/or the cellular components are removed. As used herein, the terms “transcriptomic DNA,” “tDNA,” and their equivalents, may refer to DNA molecules whose sequences are indicative of the sequences of RNA present in a cell. As used herein, the terms “spatial DNA,” “sDNA,” and their equivalents, may refer to DNA molecules whose sequences are indicative of 3D chromatin structure and genetic sequences in the cell. For instance, sDNA can be utilized in a HI-C workflow to determine 3D chromatin structure of the cell.

In various implementations, the permeabilized cells/nuclei include cross-linked chromatin and RNA. The DNA in the chromatin, as well as the RNA, may be cross-linked to cellular proteins via protein-protein, protein-DNA and protein-RNA interactions. Thus, the DNA and the RNA are fixed at their respective original position (in situ) within the nucleus. In various implementations, the tDNA may be generated by reverse transcribing the RNA in the permeabilized cells with a primer. The sDNA may be generated by fragmenting the cross-linked chromatin in the cell using at least one first restriction enzyme (RE). Proximity ligation may be performed on the fragments, such that portions of the DNA that are spatially close to one another can be ligated with each other. Subsequently, the sDNA may be generated by further fragmenting the ligated fragments using at least one second restriction enzyme (RE). The tDNA and sDNA may be reverse crosslinked. Reverse crosslinking, for instance, may set free sDNA and tDNA from cellular protein. The tDNA and sDNA may be subsequently isolated from one another into separate libraries, that can be subsequently sequenced (e.g., using nanopore sequencing, sequencing-by-synthesis, or other sequencing techniques known in the art). The sequence read data indicating the sequences of the tDNA library and the sDNA library can be further analyzed in order to determine correlations between 3D chromatin structure in the nucleus of a cell and expression by that cell, for example.

Implementations of the present disclosure are different in several ways from existing technologies. For example, the tDNA and sDNA are generated while the cellular components of the permeabilized cells are present. Thus, various steps of the protocol are performed at temperature ranges, salt concentrations, and other conditions that mimic the physiological conditions of the source of the cells. In some cases, the first RE(s) and/or second RE(s) described herein are different than enzymes used in other techniques. In various examples, techniques described herein are capable of generating tDNA and sDNA in parallel protocols, such that the resulting tDNA and sDNA libraries indicate simultaneous biological processes.

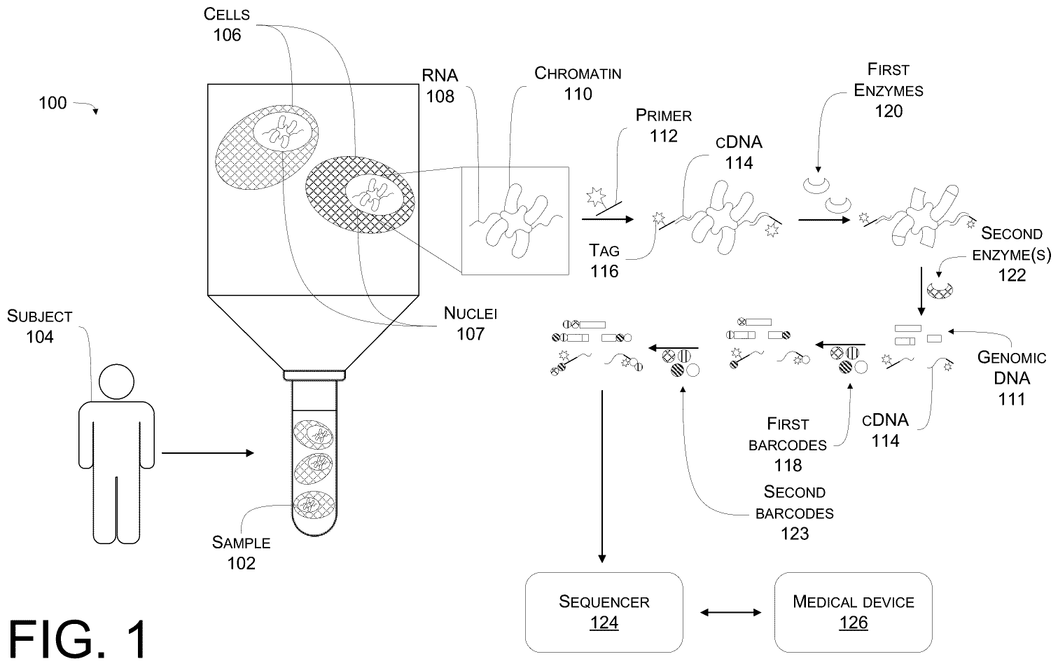

FIG. 1 illustrates an example environment 100 for characterizing the spatial genomic organization and gene expression in a sample 102. In various implementations, the sample 102 is collected from a subject 104. The sample 102 includes, in some cases, cells 106 of the subject 104. For instance, the sample 102 may be a tissue sample, a blood sample, a urine sample, or any other sample derived from the subject 104. The subject 104 may be a human, a non-human primate, a mammal, a rat, a mouse, or any other organism. In some examples, the subject 104 may suffer from a pathogenic condition. In various implementations, the sample 102 includes synthetic or artificially produced cells.

The cells 106, in various cases, include genetic material in the nuclei 107. For instance, the cells 106 may include nucleic acids, such as DNA (e.g., genomic DNA 111, exogenous DNA, or other DNA) and RNA 108 (e.g., messenger RNA (mRNA), transfer RNA (tRNA), ribosomal RNA (rRNA), and the like). In some examples, the cells 106 include chromatin 110. The chromatin 110, in various cases, includes genomic DNA 111 and at least one protein (e.g., histones, such as Histone H2A, Histone H2B, Histone H1, Histone H3, Histone H4, etc.).

The genomic DNA 111, in some examples, wraps around histone proteins to form a nucleosome. The nucleosome structure can be, in various cases, stabilized by additional histone proteins (e.g., H1), and the stabilized structure, for instance, may coil to form a compact structure. Accordingly, the genomic DNA 111 can be accessed, based on the structure of the histones, and used to generate the RNA 108 through the process of transcription, which is indication of various expressive characteristics of the subject 104. Accessing the genomic DNA 111 from chromatin to generate the RNA 108 depends on various factors that are independent of the DNA sequence, including the chromatin structure, various chromatin remodeling complexes, histone modifications, and other factors. These factors have been implicated in a variety of pathologies, including cancer, neurological diseases, cardiovascular diseases, inflammatory and autoimmune diseases, and development disorders, among others.

In various implementations, it may be beneficial to determine the spatial organization and the gene expression of the genetic material of the subject 104. For instance, it may be beneficial to understand the relationship between the three-dimensional organization of the genome in a single cell and the transcriptional activities in the single cell. In some cases, understanding the relationship may facilitate the development of diagnostic, research, and therapeutic tools. These issues can be addressed, in some implementations, by using various methods described herein to characterize the spatial organization of the genome and the gene expression within single cells.

In various implementations of the present disclosure, the cells 106 and/or the nuclei 107 in the sample 102 are crosslinked and permeabilized. For example, the chromatin 110 in the sample 102 may be crosslinked to preserve the spatial organization. Crosslinking may be achieved, in various cases, chemically (e.g., using formaldehyde, or the like) or using another suitable method. Based on crosslinking the cells 106 or the nuclei 107, the cells 106 may be permeabilized via fixation (e.g., acetone fixation, methanol fixation, or the like), using a detergent, or another method known in the art. In some examples, the cells 106 are permeabilized and the nuclei 107 are intact.

The RNA 108 in the permeabilized cells 106 or nuclei 107 is, in some examples, reverse transcribed with a primer 112 that is configured to generate cDNA 114 that includes a tag 116. In various cases, the primer 112 is a poly-thymine (poly-T) primer. The primer 112, in some instances, is a random hexamer primer. The tag 116 may include a biotin, an avidin, a polyhistidine tag, a m6A methyl, an amine group, or the like. For instance, the primer 112 may include a biotinylated nucleotide. The tag 116 may be configured to ligate with first barcodes 118.

In various implementations, the chromatin 110 in the permeabilized cells 106 or nuclei 107 is fragmented using first enzymes 120. The first enzymes 120 are configured to fragment the chromatin 110 to facilitate proximity ligation of the chromatin 110. The first enzymes 120, in some cases, include two four-cut restriction enzymes. In particular examples, the first enzymes 120 include CviQI and MseI. Applying the first enzymes 120, in various cases, is performed at a temperature in a range of 20 degrees Celsius (20° C.) to 30° C. In some cases, the first enzymes 120 are configured to generate a thymine-adenine (−TA) at the 5′ end of the fragmented chromatin 110. Based on the fragmentation, the chromatin 110 may undergo proximity ligation, enabling identification of chromatin interactions based on the ligated DNA sequence. In various cases, the proximity ligation may be performed at a temperature in a range of 10° C. to 20° C.

In various implementations, the chromatin 110 is fragmented using at least one second enzyme 122. The second enzyme(s) 122, in some examples, are configured to fragment the chromatin 110 to generate an adhesive end of the fragmented chromatin 110, enabling ligation to the first barcodes 118. In various examples, the second enzyme(s) 122 include a restriction enzyme, such as Ddel, or any other suitable enzyme. Applying the second enzyme(s) 122, in various cases, is performed at a temperature in a range of 30° C. to 40° C.

Based on the fragmentation with the second enzymes 122, the first barcodes 118 are applied to the sample 102. The first barcodes 118, in some cases, are configured to ligate to the cDNA 114 and the genomic DNA 111 derived from the fragmented chromatin 110. The first barcodes 118, in various examples, include a plurality of polynucleotides. The DNA sequences may, for instance, have a length in a range of 2 to 20 nucleotides. A well plate, a microfluidic devices, tubes, or another mechanism may be used to separate the sample into first volumes. One of the first barcodes 118 may be added to each of the first volumes. For instance, the sample 102 may be added to a 96-well plate, and a distinct barcode may be added to each of 96 wells. In some examples, the same first barcode(s) 118 may be added to more than one of the 96 wells.

After applying the first barcodes 118, in various implementations, second barcodes 123 are applied to the sample 102. The second barcodes 123, in some cases, are configured to ligate to at least one of the first barcodes 118, the cDNA 114, or the genomic DNA 111. For instance, based on applying the first barcodes 118 to the first volumes, the sample 102 may be pooled from the first volumes and redistributed into second volumes. A well plate, a microfluidic devices, tubes, or another mechanism may be used to separate the sample into second volumes. The second barcodes 123, in some cases, include a plurality of polynucleotides. The DNA sequences may, for instance, have a length in a range of 2 to 20 nucleotides. In some examples, the first barcodes 118 and the second barcodes 123 are the same. In some examples, some of the sequences of the first barcodes 118 are the same as some of the sequences of the second barcodes 123.

In response to applying the second barcodes 123, the cDNA 114 may be separated from the genomic DNA 111 using the tag 116. For instance, the sample 102 may be pooled from the second volumes, and the sample 102 may undergo reverse crosslinking. The reverse crosslinking may be achieved using proteinase K, or another suitable technique. After the reverse crosslinking process, the cDNA 114 may be separated from the genomic DNA 111 using a complementary tag. For instance, the cDNA 114 may include biotin, and the complementary tag may include streptavidin. The complementary tag may include any agent configured to bind to the tag. For instance, the complementary tag may include streptavidin, biotin, avidin, nitrilotriacetic acid, or the like. The complementary tag may be linked to beads, a surface, a support, or the like that is configured to be isolated from the sample 102. For instance, the sample 102 may be applied to magnetic beads that are conjugated to the complementary tag, and the beads may be isolated from the sample 102 by applying a magnetic field to the sample 102 to isolate the cDNA 114. The sample 102 that remains, in various examples, includes the genomic DNA 111.

In particular implementations, additional barcodes may be applied. For instance, the second volumes may be pooled and separated into third volumes. Third barcodes may be applied to each of the third volumes. In various examples, three, four, five, six, or more than six barcodes may be applied. The number of barcodes, in some cases, may be determined based on the volume of the sample 102 or the number of cells 106 in the sample 102.

A sequencer 124, in various implementations, generates a transcriptomic library and a spatial library by sequencing the cDNA 114 and the genomic DNA 111, respectively. The transcriptomic and spatial libraries may be sequenced, and the first barcodes 118 and second barcodes 123 may be used to determine which of the sequences are associated (e.g., from the same cell, cell population, physical region of a sample, etc.). The sequencing technique may include at least one of a massively parallel sequencing (MPS) technique, next generation sequencing, targeted sequencing, direct sequencing, Sanger sequencing, sequencing-by-synthesis, nanopore sequencing on the tDNA library, or any other suitable nucleic acid sequencing technique.

In some implementations, at least some of the methods and/or reagents described herein may be incorporated into a medical device 126. For instance, the medical device 126 may include reservoirs configured to hold at least one of the sample 102, the primer 112, the first enzymes 120, the second enzyme(s) 122, the first barcodes 118, the second barcodes 123, or any other reagent used in the methods described herein. In some examples, the medical device 126 is configured to introduce a reagent to the sample 102. For example, the medical device 126 may be configured to introduce the sample 102 and the tagged primer 112, the first enzymes 120, the second enzyme(s) 122, or any other reagent described herein to a container. In some examples, the medical device 126 is configured to separate the sample 102 into the first volumes and/or the second volumes. The medical device 126 may be configured to introduce the first barcodes 118 to the first volumes and/or the second barcodes 123 to the second volumes. In some examples, the medical device 126 may be configured to sequence the cDNA 114 and/or the genomic DNA 111. The medical device 126 may be configured to analyze the first and second barcodes 118 and 123 to determine the sequences corresponding to a single cell, a cell population, or the like. In various examples, the medical device 126 may be configured to analyze the transcriptomic and spatial libraries and output a report to a user (e.g., a laboratory technician, a researcher, a trained user, a clinician, a nurse, or the like) or an external device. The medical device 126, in some examples, includes a fluidic device, a microfluidic device, a robotic device, a computer, a processor, the sequencer 124, or any other device that can execute the methods described herein.

In some implementations of the present disclosure, spatial barcodes that are associated with a physical location within the sample 102 are added to the sample 102. For example, the sample 102 may include a tissue slice, and each of the spatial barcodes may be applied to distinct physical regions of the tissue slice. Based on applying the spatial barcodes, the sample 102 may be undergo the processes described herein to generate the transcriptomic library and the spatial library. In various implementations, the physical distribution of the transcriptomic and spatial libraries may be determined using the spatial barcodes.

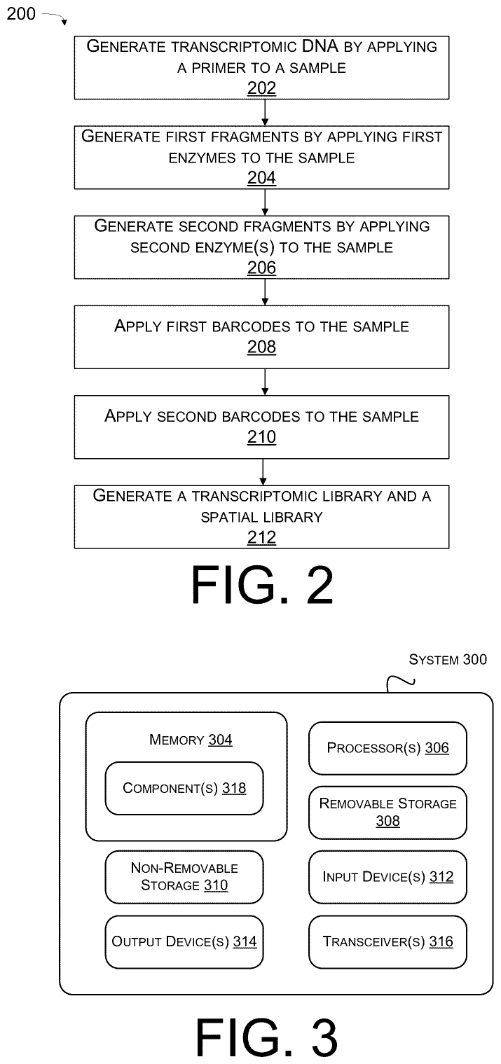

FIG. 2 illustrates an example process 200 for characterizing the spatial genomic organization and the gene expression in single cells. In various implementations, the process 200 may be performed by an entity (e.g., a laboratory technician, a researcher, a trained user, or the like) and/or a device (e.g., a computer, a fluidic device, a robotic device, or the like). In various implementations, some or all of the steps of the process 200 may be omitted.

At 202, transcriptomic DNA is generated by applying a primer (e.g., the primer 112) to a sample (e.g., the sample 102). In various examples, the sample includes cells (e.g., the cells 106). The cells may be derived from a subject, or the cells may include synthetic cells. The sample may include permeabilized cells and crosslinked chromatin (e.g., the chromatin 110). In some implementations, the process 200 may include permeabilizing the cells and/or the nuclei (e.g., the nuclei 107) of the cells. In some implementations, the process 200 may include crosslinking the chromatin in the cells. The primer, in various examples, is configured to generate tagged cDNA (e.g., the cDNA 114) by reverse transcribing RNA in the cell. The cDNA may include a tag (e.g., the tag 116) that is configured to isolate the cDNA from the sample. The primer, in some instances, is configured to facilitate the ligation of first barcodes (e.g., the first barcodes 118) to the cDNA.

At 204, first fragments of genomic DNA (e.g., the genomic DNA 111) are generated by applying first enzymes (e.g., the first enzymes 120) to the sample. In various cases, the first enzymes are configured to fragment the crosslinked chromatin in the sample. In particular implementations, the first enzymes include two four-cut restriction enzymes (e.g., Msel, CviQI, or the like). In various examples, the first fragments include a TA at the 5′ end. The first fragments may be ligated by performing proximity ligation.

At 206, second fragments are generated by applying at least one second enzyme (e.g., the second enzyme(s) 122) to the sample. In some examples, the second enzyme(s) are configured to facilitate the ligation of first barcodes to the second fragments.

At 208, the first barcodes are applied to the sample. In some examples, the sample is separated into first volumes and each of the first barcodes are applied to the first volumes. A particular barcode of the first barcodes may be applied to each of the first volumes. In various cases, a particular barcode of the first barcodes may be applied to more than one of the first volumes. Based on applying the first barcodes, the first volumes may be pooled into the sample.

At 210, second barcodes (e.g., the second barcodes 123) are applied to the sample. In some examples, the sample is separated into second volumes and each of the second barcodes are applied to the second volumes. A particular barcode of the second barcodes may be applied to each of the second volumes. In various cases, a particular barcode of the second barcodes may be applied to more than one of the second volumes. Based on applying the second barcodes, the second volumes may be pooled into the sample.

At 212, a transcriptomic library and a spatial library of the sample are generated. For example, based on applying the second barcodes, the chromatin in the sample may be reverse crosslinked to generate genomic DNA sequences that include the first and second barcodes. The cDNA that includes the first and second barcodes, in some cases, is isolated from the sample by using a complementary tag. The complementary tag, in various examples, is configured to bind to the tag. For instance, a complementary tag may be linked to a magnetic bead and applied to the sample. A magnetic field may be applied to the sample to isolate the magnetic beads, thereby isolating the cDNA from the genomic DNA sequences. Based on isolating the cDNA, the transcriptomic library can be generated by sequencing the cDNA, and the spatial library can be generated by sequencing the genomic DNA sequences (e.g., by using the sequencer 124). In various implementations, the associated cDNA and genomic DNA sequences (e.g., from the same cell, cell population, physical region of the sample, etc.) can be determined using the first and second barcodes.

FIG. 3 illustrates an example of a system 300 for performing various functions described herein. In some cases, the system 300 can represent the medical device 126 described above with reference to FIG. 1.

As illustrated, the system 300 can include a memory 302. In various implementations, the memory 302 is volatile (including a component such as Random Access Memory (RAM)), non-volatile (including a component such as Read Only Memory (ROM), flash memory, etc.) or some combination of the two. The memory 302 may include various data, such as at least one component 304. The component(s) 304 can include methods, threads, processes, applications, or any other sort of executable instructions. For instance, the component(s) 304 may include instructions for performing any of the functionality described above with reference to FIGS. 1-2. The instructions, and various other elements stored in the memory 302, can also include other files and databases.

The memory 302 may include various instructions (e.g., among the component(s) 304), which can be executed by at least one processor 306 to perform operations. In some implementations, the processor(s) 306 includes a Central Processing Unit (CPU), a Graphics Processing Unit (GPU), or both CPU and GPU, or other processing unit or component known in the art.

The system 300 can also include additional data storage devices (removable and/or non-removable) such as, for example, magnetic disks, optical disks, or tape. Such additional storage can include removable storage 308 and non-removable storage 310. Tangible computer-readable media can include volatile and nonvolatile, removable and non-removable media implemented in any method or technology for storage of information, such as computer readable instructions, data structures, program modules, or other data. The memory 302, removable storage 308, and non-removable storage 310 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the system 300. Any such tangible computer-readable media can be part of the system 300.

The system 300 also can include input device(s) 312, such as a keypad, a cursor control, a touch-sensitive display, voice input device, etc., and output device(s) 314 such as a display, speakers, printers, etc. In various implementations, the input device(s) 312 can include the sequencer 124 and/or the medical device 126. These devices are well known in the art and need not be discussed at length here. In particular implementations, a user can provide input to the system 300 via a user interface associated with the input device(s) 312 and/or the output device(s) 314.

The system 300 can also include one or more wired or wireless transceiver(s) 316. For example, the transceiver(s) 316 can include a Network Interface Card (NIC), a network adapter, a Local Area Network (LAN) adapter, or a physical, virtual, or logical address to connect to the various base stations or networks contemplated herein, for example, or the various user devices and servers. To increase throughput when exchanging wireless data, the transceiver(s) 316 can utilize Multiple-Input/Multiple-Output (MIMO) technology. The transceiver(s) 316 can include any sort of wireless transceivers capable of engaging in wireless, Radio Frequency (RF) communication. The transceiver(s) 316 can also include other wireless modems, such as a modem for engaging in Wi-Fi, WiMAX, Bluetooth, or infrared communication.

In some implementations, the transceiver(s) 316 can be used to communicate between various functions, components, modules, or the like, that are included in the system 300. For instance, the transceiver(s) 316 can be used to transmit data between the system 300 and the sequencer 124, the medical device 126, an external user equipment (UE), analysis device, or the like.

Implementations of the present disclosure will now be described with reference to an Experimental Example.

### EXPERIMENTAL EXAMPLE

This Experimental Example describes GAGE-seq (genome architecture and gene expression by sequencing), a highly scalable and cost-effective method for simultaneously profiling of chromatin interactions and gene expression in single cells. GAGE-seq, due to its combinatorial barcoding strategy, offers higher methodological throughput, as well as greater efficiency and effectiveness than recent technologies such as HiRES (Liu Z, et al. Science 2023; 380:1070-6). GAGE-seq was applied to profile 9,190 cells across diverse mammalian cell lines and tissues, including mouse brain and human bone marrow. Specifically, an experimental and analytical framework was developed to elucidate the connections between multiscale 3D genome features and cell type-specific gene expression, as well as their spatial and temporal interplay.

### Materials and Methods.

### Ethics Statement.

The present study complies with all pertinent ethical regulations. All the mice used in this study received humane care in compliance with the principles stated in the Guide for the Care and Use of Laboratory Animals, NIH Publication, 1996 edition, and the protocols were approved by the Institutional Animal Care Committee (IACUC) at the University of Washington (Seattle, WA).

Cell lines used.

K562 (#CCL-243, ATCC), GM12878 (#GM12878, Coriell) and NIH3T3 cells (CRL-1658, ATCC) were purchased from the respective vendors. The myelodysplastic cell line MDS-L was a gift from Dr. Kaoru Tohyama (Kawasaki University of Medical Welfare, Japan).

GAGE-seq experimental details.

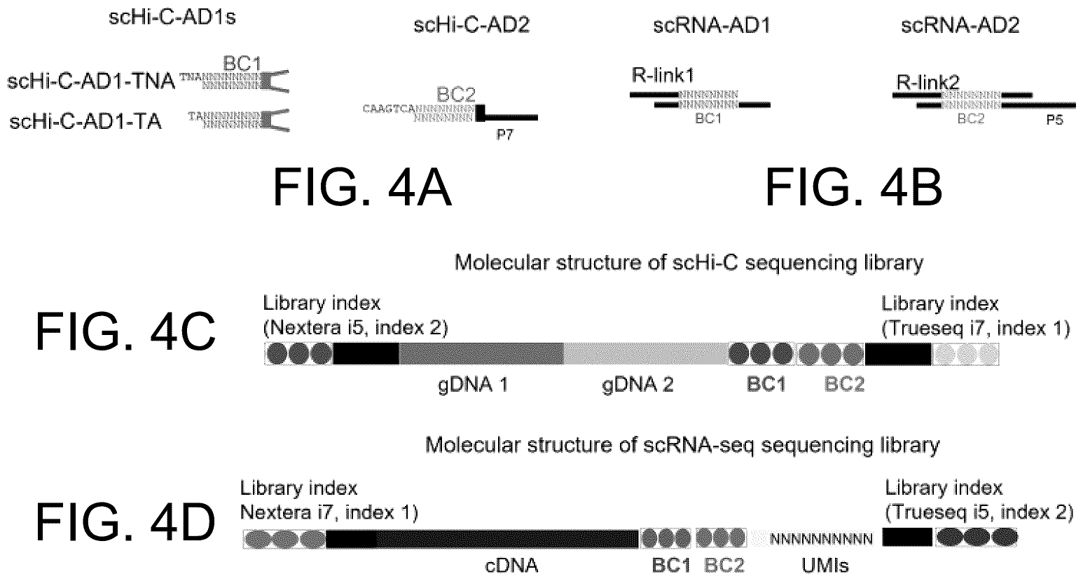

FIGS. 4A-4D illustrate the molecular design of GAGE-seq adaptors and the molecular structure of the DNA fragments in GAGE-seq scHi-C and scRNA libraries. FIGS. 4A, 4B illustrate the structure of the two-round barcoded adaptors used in scHi-C (4A) and scRNA-seq (4B). The molecular structure of the four adaptors is similar, with each of them containing three different functional parts. For the scHi-C-AD1 adaptor, (i) its 5′-sticky ends (5′-TNA or 5-TA) are designed to be compatible with the 5′-ends of genomic DNA fragments generated by Ddel or CviQI/Msel digestion; (ii) the middle sequence is the 1st-round barcodes (BC1); and (iii) its 3′-end is Y-shaped, with its two strands possessing different functions. One strand contains the Illumina Nextera i5 sequence, designed for read 1 and index 2 sequencing of the Hi-C libraries, and the other strand provides the sequence TGACTTG for ligating the scHi-C-AD2 adaptor. For the scHi-C AD2 adaptor, (i) its 5′-CAAGTCA end is for ligating with the scHi-C-AD1 adaptors; (ii) its middle sequence is the 2nd-round barcodes (BC2); and (iii) its 3′-end contains the Illumina trueseq i sequence for read 2 and index 1 sequencing of the Hi-C libraries. For the scRNAAD1 adaptor, (i) its 5′-R-link1 end is for ligating with the cDNAs; (ii) its middle sequence is the 1stround barcodes (BC1); and (iii) its 3′-end sequence is for ligating with the scRNA-AD2 adaptor. For the scRNA-AD2 adaptor, (i) its 5′-R-link2 end is for ligating with the scRNA-AD1 adaptors; (ii) its middle sequence is the 2nd-round barcodes (BC2); and (iii) its 3′-end contains the Illumina trueseq i5 sequence for read 1 and index 2 sequencing of the RNA libraries. (FIGS. 4C, 4D) illustrate the overall molecular structure of DNA fragments in GAGE-seq scHi-C (4C) and scRNA-seq (4D) libraries. The DNA fragments in the two library types are structurally designed to use different sequencing primers for NGS, effectively preventing cross-contamination between them.

Preparation of the 96-well plates of barcoded adaptors. Two separate barcoding rounds of ligation reactions are used in GAGE-seq. The design of the scRNA-seq part barcodes resembles that of Split-seq (Rosenberg A B, et al. Science 2018; 360:176-82) and SHARE-seq (Ma S, et al. Cell 2020; 183:1103-1116). The molecular structure of the scHi-C part barcodes is depicted in (FIGS. 4A-4D).

Cell lysis. Crosslinked cells of K562, NIH3T3, GM12878, MDS-L, human bone marrow Cd34+ cells were thawed from −80° C. or liquid nitrogen. 0.2 ml of high-salt lysis buffer 1 (50 mM HEPES pH 7.4, 1 mM EDTA pH 8.0, 1 mM EgTA pH 8.0, 140 mM NaCl, 0.25% Triton X-100, 0.5% IGEPAL CA-630, 10% glycerol, and 1× proteinase inhibitor cocktail (PIC)) was added per 1×10⁶ cells. The cell solution was mixed thoroughly and incubated on ice for 10 min. After this, cells were pelleted at 500×gravity (×g) for 2 min at 4° C. and then resuspended in 0.2 ml of high-salt lysis buffer 2 (10 mM Tris-HCl PH 8, 1.5 mM EDTA, 1.5 mM EgTA, 200 mM NaCl, 1×PIC). The solution was incubated on ice for 10 min. Following this, cells were then pelleted at 500×g for 2 min at 4° C. and then resuspended in 200 ul of 1×T4 DNA ligase buffer (NEB, B0202S) containing 0.2% SDS. They are then incubated at 58° C. for 10 min. To quench the reaction, 200 μL ice-cooled 1×NWB and 10 μl 10% Triton X-100 (MilliporeSigma, 93443) were added to the tube. Finally, cells were spun at 500×g for 4 min at 4° C. For crosslinked mouse brain cortex cells, the treatment was simplified. The step involving high-salt lysis buffer 1 and high-salt lysis buffer 2 was omitted, and 0.1% SDS was used for cell lysis.

Reverse transcription. SDS treated cells were resuspended in 400 μL of RT mix (final concentration of 1×RT buffer, 500 mM dNTP, 10 mM Biotinylated RT primers, 7.5% PEG 6000 (VWR, 101443-484), 0.4 U/ml SUPERase⋅In™ RNase Inhibitor, and 25 U/ml Maxima H Minus Reverse Transcriptase (ThermoFisher Scientific of Waltham, MA, EP0752)). The RT primers contain a poly dT tail, a biotin molecule, and a universal ligation overhang. The sample then underwent a series of heating cycles. Initially, it was heated at 50° C. for 10 minutes, then it went through 3 thermal cycles (8° C. for 12 s, 15° C. for 45 s, 20° C. for 45 s, 30° C. for 30 s, 42° C. for 2 min and 50° C. for 3 min). Afterwards, the sample was again incubated at 50° C. for 10 minutes. After reverse transcription, 600 μL of 1×NWB was added, the sample was centrifuged at 500×g for 3 minutes, and the supernatant was then removed.

1st-round chromatin fragmentation, proximity ligation, and 2nd-round chromatin fragmentation. Cells were resuspended in 400 μL of restriction enzyme (RE) digestion mix (1×T4 ligase buffer (New England Biosciences (NEB) of Ipswich, MA, B0202S), 500 U Msel (NEB, R0525M), 240 U CviQI (NEB, R0639L), 0.32 U/mL Enzymatics RNase Inhibitor, 0.05 U/ml SUPERase RNase Inhibitor), and incubated at room temperature (25° C.) for 2 hr. Cells were then centrifuged at 500×g for 3 minutes at 4° C., and the supernatant was removed. The remaining cell pellet was washed twice with 300 μL of 1×NWB, and as much supernatant was removed as possible. Next, the pellet was resuspended in 200 μL of ligation mix (1×T4 ligation buffer (NEB, B0202S), 50 Units T4 DNA ligase (ThermoFisher Scientific, EL0012), 0.32 U/ml Enzymatics RNase Inhibitor, 0.05 U/ml SUPERase RNase Inhibitor) and incubated at 16° C. overnight. This was followed by adding 20 μL 10×T4 ligation buffer, 1 μL SUPERase RNase Inhibitor and 20 μL Ddel (NEB, R0175L). The sample was then incubated at 37° C. for 1 hr and centrifuged at 500×g for 3 minutes, with the supernatant removed afterwards.

Combinatorial cellular barcoding. Cells were resuspended in 330 μL of ligation mix (1×T4 ligase buffer (NEB, B0202S), 100 Units T4 DNA ligase (ThermoFisher Scientific, EL0012), 0.25 mg/ml BSA (ThermoFisher Scientific, AM2618), 5% PEG-4000 (ThermoFisher Scientific, EL0012), 0.32 U/ml Enzymatics RNase Inhibitor, 0.05 U/ml SUPERase RNase Inhibitor) and distributed into each well (3 μL/well) of the first-round barcoding plate, which already contained 2 μL of CARE-seq 1st-round adaptors in each well. This barcoding plate was then incubated at 25° C. for 3 hr. Afterwards, cells from all 96 wells were pooled into three 1.5 ml tubes, and 5 μl of 10% NP-40 (ABCam of Waltham, MA, ab142227) was added to each tube. This is followed by centrifuging at 500×g for 3 minutes at 4° C. The supernatant was then removed and cells were resuspended in 300 μL 1×NWB containing 0.033% SDS and combined into one 1.5 ml tube. Cells were then pelleted at 500×g for 2 minutes at 4° C. After three additional rounds of washing with 300 μL 1×NWB containing 0.033% SDS, cells were resuspended in 200 μl 1×NWB containing 0.1% SDS and filtered with with 10 μm or 20 μm cell ministrainer (PluriStrainer, pluriSelect of Leipzig, Germany, 43-10010-50 or 43-10020-40). Cells were inspected under a microscope and counted with a hemocytometer. 7,500 cells were diluted with 1.25 ml of a dilution buffer containing 0.4×NEBuffer 2 (NEB, B7002S), 2 mg/ml BSA (ThermoFisher Scientific, AM2618), and 0.08 UM RNA ligation-1 block, and distributed into each well (3 μL/well) of a 96-well plate (the 2nd-round barcoding plate). Then, 2 MI of cell lysis buffer (5×NEBuffer 2, 0.625% SDS) were then added to each well of the 2nd-round barcoding plate. The plate was incubated at 60° C. for at least 24 hr.

For the 2nd-round barcoding, 1.5 μL of pre-mixed GAGE-seq adapters (0.2 μM Hi-C-AD2 and 0.17 μM RNA-AD2) were added to the plate, followed by 23.5 μL of ligation mix (3 μL 1×T4 ligase buffer (NEB, B0202S), 0.15 μL 50 mg/ml BSA (ThermoFisher Scientific, AM2618), 1 μL 10% Triton X-100 (MilliporeSigma, 93443), 0.03 μL 20 μM 5′-P-TNA-Nextera-P5-AD, 0.03 μL 20 μM 5′-P-TA-Nextera-P5-AD, 0.03 μl 10 μM RNA ligation-1 block, and 0.8 μl T4 DNA ligase (ThermoFisher Scientific, EL0012)). The ligation was carried out at 25° C. for 24 hr, and then stopped by adding 2 μL of proteinase K digestion mix (0.2 μL proteinase K (ThermoFisher Scientific, AM2546), 0.5 μL 10% SDS and 1.8 μL water) to each well. A reverse crosslinking was carried out at 60° C. for 20 hr.

Reverse crosslinking and separation of scHi-C and scRNA-seq libraries. After reverse crosslinking, the sample in each 96-well plate was pooled into 12 DNA low-binding 1.5 ml tubes (Eppendorf of Hamburg, Germany, 022431021). Genomic DNA (gDNA) and cDNA were precipitated by adding 66 μL 3M Sodium Acetate Solution (pH 5.2) (MilliporeSigma of Burlington, MA, 127-09-3), 1 μL GlycoBlue (ThermoFisher Scientific, AM9515) and 720 μl iso-propanol (MilliporeSigma, 19516) to each tube, followed by incubating at −80° C. for at least 1 hr. The samples were then centrifuged at 15000 rotations per minute (rpm) for 10 min and the pellet in each tube were resuspended in 30 μL 1×NEBuffer 2 containing 0.15% SDS. After incubation at 37° C. for 10 min, the samples were combined into one DNA low-binding tube. gDNA and cDNA were precipitated by adding 66 μL 3M Sodium Acetate Solution (pH 5.2) and 720 μL iso-propanol, followed by incubating at −80° C. for at least 1 hr. The sample was then centrifuged at 15000 rpm for 10 min and the pellet was resuspended in 100 μl buffer EB (Qiagen of Hilden, Germany, 19086). For each sample of a 96-well plate, 5.5 μL of MyOne C1 Dynabeads were washed twice with 1×B&W-T buffer (5 mM Tris pH 8.0, 1M NaCl, 0.5 mM EDTA, and 0.05% Tween 20) and resuspended in uL of 2×B&W buffer (10 mM Tris pH 8.0, 2M NaCl, and 1 mM EDTA) and added to the sample tube. The mixture was incubated at room temperature for 60 min and put on a magnetic stand to separate supernatant and beads.

Library construction and sequencing. Both scHi-C and scRNA-seq libraries were pooled and paired end sequencing (PE 150) were performed on the HiSeq, NextSeq, or NovaSeq platform (Illumina).

### GAGE-Seq Data Processing Workflow.

Demultiplexing. DNA and RNA reads were assigned to wells based on the two rounds of barcodes. For DNA reads, only read 2 was used for demultiplexing, allowing at most 1 mismatch in each of the two rounds of barcodes. DNA reads with more than 5 mismatches in the region between the two rounds of barcodes (the 9th-23rd nucleotides (nt)) were discarded. After demultiplexing, the first 12 nt were removed from read 1 and the first 35 nt were removed from read 2. For RNA reads, only read 1 was used for demultiplexing, allowing at most 1 mismatch in each barcode round. RNA reads with more than 6 mismatches in the region between the two rounds of barcodes (the 19th-48th nt) or with more than 6 mismatches in the region downstream of the first round of barcode (the 57th-71th nt) were discarded.

The two reference genomes were combined into a single reference genome file used for all GAGE-seq libraries. For DNA reads, Burrows-Wheeler Aligner (BWA) (0.7.17) (Li H, Durbin R. Bioinformatics 2009; 25:1754-60) was used for alignment. The combined reference genome was indexed using command bwa index-a bwtsw. Paired, trimmed DNA reads were aligned to the combined reference genome using command bwa mem-SP5M. For RNA reads, Spliced Transcripts Alignment to a Reference (STAR) (2.7.8a) (Dobin A, et al. Bioinformatics 2013; 29:15-21) was used for alignment. The GENCODE annotation files for human (v36) and mouse (vM25) were downloaded and concatenated. The combined reference genome was indexed using command--runMode genomeGenerate--sjdbOverhang 100 with the combined gencode annotation file. Only read 2 of RNA reads was aligned with the command STAR--outSAMunmapped Within.

Identification of contact pairs from DNA reads. Pairtools (0.3.1.dev1) (Goloborodko A, et al. mirnylab/pairtools: v0.2.0. 2018) was used to identify contact pairs from paired DNA reads with command pairtools parse--walks-policy all--no-flip--min-mapq=10. After that, walk reads (i.e., DNA reads containing multiple ligation sites) were further processed. In this Example, it was assumed that any pair of loci in the same DNA read forms a valid contact pair, and these contact pairs were included in the results.

Deduplication of contact pairs. The contact pairs were deduplicated. The genomic positions of the two ends of each contact pair was extracted. Two contact pairs are defined as directly duplicated if the two contact pairs' first ends lie within 500 nt apart and their second ends also within 500 nt. If two contact pairs are not directly duplicated, but are directly or indirectly duplicated with a third contact pair, the first two contact pairs are defined as indirectly duplicated. Among each cluster (i.e., connected component) of (in) directly duplicated contact pairs, the one with the largest difference between its two ends' genomic positions was retained, and the rest were marked as duplicates.

Deduplication of RNA reads. The RNA reads were deduplicated. Two RNA reads are defined as directly duplicated if there is at most 1 mismatch in their UMI and if their genomic positions differ by at most 5 nt. The rest of the process is similar to the deduplication of contact pairs. Only one RNA read from each duplicate cluster is retained.

### GAGE-Seq Integrative Analysis for Mouse Brain Cortex.

Integration with Multiplexed Error-Robust Fluorescence in situ Hybridization (MERFISH) data. Integration of GAGE-seq data and MERFISH data was done with Seurat (4.1.1) (Chidester B, et al. Nat Genet 2023; 55:78-88). Only scRNA-seq profiles from the GAGE-seq data were used for this integration. In the GAGE-seq mouse brain cortex data, the following cell types of excitatory neurons were used: L2/3 IT CTX a, L2/3 IT CTX b, L2/3 IT CTX c, L4 IT CTX, L4/5 IT CTX, L5 IT CTX, L6 IT CTX, L6 CT CTX a, L6 CT CTX b, L5/6 NP CTX, and L6b CTX. In the MERFISH data, cells from L2/3 IT, L4/5 IT, L5 IT, L5/6 NP, L6 CT, L6 IT, and L6b were used. Each time, the selected cells from GAGE-seq were integrated with one slice from the MERFISH data. All genes detected and expressed in both GAGE-seq and MERFISH were used. The ‘FindIntegrationAnchors’ and ‘IntegrateData’ functions were used with default parameters, except that the number of dimensions was set to 10.

Inference of whole-transcriptome expression and 3D genome features for MERFISH cells. The integrated single-cell expression profiles of GAGE-seq data and MERFISH data were scaled by the ‘ScaleData’ function from Seurat with default parameters, and the first 30 PCs were calculated by the ‘RunPCA’ function. A 50-nearest neighbor regressor was created to estimate whole-transcriptome expression and 3D genome features from the 30-dimensional PC space. The regressor was trained on GAGE-seq data and then applied to the MERFISH data. The Gaussian kernel was used as the weight function. For each MERFISH cell, the bandwidth was defined as the 0.3 quantile of the distances to the 50 nearest neighbors.

Integration with Paired-seq data. The integration of GAGE-seq data with Paired-seq data52 was done using Seurat. Only scRNA-seq profiles from the GAGE-seq data and the Paired-seq data were used for this integration. In the GAGE-seq mouse brain cortex data, three cell types were excluded: L2 IT RvPP, L2/3 IT RSP, and L5 IT RSP. In the Paired-seq data, cells from BR_NonNeu_Endothelial, HC_ExNeu_CA1, HC_ExNeu_CA23, HC_ExNeu_DG, HC_ExNeu_Subiculum, and HC_NonNeu_Ependymal were excluded. The ‘SelectIntegrationFeatures’, ‘FindIntegrationAnchors’ and ‘IntegrateData’ functions were used with default parameters.

Inference of accessibility for GAGE-seq cells. The integrated single-cell expression profiles of GAGE-seq data and Paired-seq data were scaled by the ‘ScaleData’ function from Seurat with default parameters. The first 20 PCs were calculated by the ‘RunPCA’ function. To estimate whole-transcriptome expression and 3D genome features from the 40-dimensional PC space, a 50-nearest neighbor regressor was created, which was trained on Paired-seq data and then applied to the GAGE-seq data. The Gaussian kernel was used as the weight function. For each GAGE-seq cell, the bandwidth was set based on the 0.3 quantile of the distances to the 40 nearest neighbors.

### GAGE-Seq Integrative Analysis for Bone Marrow.

Trajectory and pseudotime. The pseudotime of human bone marrow cells was inferred by the ‘sc.tl.diffmap’ and ‘sc.tl.dpt’ function in Scanpy (1.9.3) (Wolf F A, et al. Genome Biol 2018; 19:15), jointly from the paired scRNA-seq profiles and scHi-C profiles. Specifically, cells in the HSC, MPP, MLP, and B-NK clusters were included. The first 5 PCs of the scRNA-seq profiles were used for the scRNA-based pseudotime and the first 2 PCs of the Fast-Higashi embeddings of the scHi-C profiles were used for the scHi-C-based pseudotime. The 5 scRNA-seq PCs and the 2 scHi-C PCs were then concatenated and used for the joint pseudotime. The ‘sc.pp.neighbors’ function was used to construct the neighbor graph with 30 (scRNA-based and joint pseudotime) or 20 (scHi-C-based pseudotime) nearest neighbors per cell. The ‘sc.tl.diffmap’ and ‘sc.tl.dpt’ function was applied with 10 diffusion components to learn a latent representation focusing on the trajectory and to infer the pseudotime for single cells. The origin of the trajectory was set based on the average expression level of HSC marker genes previously identified (Zhang Y, et al. Dev Cell 2022; 57:2745-60).

Unsupervised clustering of genes. The clustering of genes was based on the expression and scA/B value. Genes expressed in at least 20 cells were included. To generate features for genes, 1) the expression levels and scA/B values were z-score normalized per gene among all cells. 2) cells were evenly divided into 10 bins based on the pseudotime, and 3) the average values of the expression and scA/B value in each bin were calculated for each gene. This process led to 20 features for each gene. The Louvain clustering algorithm was then applied to genes with 20 neighbors, a resolution of 1.5. The correlation was used as the distance metric.

### Statistics and Reproducibility.

Boxplots in all figures show the median, first, and third quartiles, and whiskers extend no further than 1.5× interquartile range. The robustness and reproducibility of GAGE-seq were validated extensively by using multiple cell lines and primary tissue cells (both mouse and human). Blinding was not relevant to the study, thus data collection and analysis were not performed blind to the conditions of the experiments. No statistical method was used to predetermine sample size. The experiments were not randomized.

### Data Availability.

All sequencing data from this study have been submitted to GEO under the accession #GSE238001. The following publicly available datasets were used in this Example: in situ Hi-C datasets from Rao et al. (Cell 2014; 159:1665-80) (GSE: GSE63525); scHi-C datasets from Nagano et al. (Nature 2013; 502:59-64) (GEO: GSE48262), Nagano et al. (Nature 2017; 547:61-7) (GEO: GSE94489), Ramani et al. (Nat Methods 2017; 14:263-6) (GEO: GSE84920), Kim et al. (PLOS Comput Biol 2020; 16) (4DN Data Portal: 4DNES4D5MWEZ, 4DNESUE2NSGS, 4DNESIKGI39T, 4DNES1BK1RMQ, and 4DNESTVIP977), Tan et al. (Science 2018; 361:924-8) (GEO: GSE117876), Tan et al. (Nat Struct Mol Biol 2019; 26:297-307) (GEO: GSE121791), Tan et al. (Cell 2021; 184:741-758) (GEO: GSE162511), Flyamer et al. (Nature 2017; 544:110-4) (GEO: GSE80006), Gassler et al. (EMBO J 2017; 36:3600-18) (GEO: GSE100569), Stevens et al. (Nature 2017; 544:59-64) (GEO: GSE80280), Collombet et al. (Nature 2020; 580:142-6) (GEO: GSE129029), Lee et al. (Nat Methods 2019; 16:999-1006) (GEO: GSE124391), Liu et al. (Nature 2021; 598:120-8) (GEO: GSE132489), and Mulqueen et al. (Nat Biotechnol 2021; 39:1574-80) (GEO: GSE174226); scRNA-seq datasets from Chen et al. (Nat Biotechnol 2019; 37:1452-7) (GEO: GSE126074), Plongthongkum et al. (Nat Protoc 2021; 16:4992-5029) (GEO: GSE157660), Chen et al. (Nat Methods 2022; 19:547-53) (GEO: GSE178707), Ma et al. (Cell 2020; 183:1103-1116) (GEO: GSE140203), Xu et al. (Nat Methods 2022; 19:1243-9) (ArrayExpress: E-MTAB-11264), Xiong et al. (Nat Methods 2021; 18:652-60) (GEO: GSE158435), Zhu et al. (Nat Struct Mol Biol 2019; 26:1063-70) (GEO: GSE130399), Zhu et al. (Nat Methods 2021; 18:283-92) (GEO: GSE152020), Cao et al. (Science 2018; 361:1380-5) (GEO: GSE117089), Mimitou et al. (Nat Methods 2019; 16:409-12) (GEO: GSE126310), and Zhang et al. (Dev Cell 2022; 57:2745-2760) (GEO: GSE137864); HiRES co-assayed scHi-C and scRNA-seq datasets from Liu et al. (Science 2023; 380:1070-6) (GEO: GSE223917); MERFISH spatial transcriptome datasets from Zhang et al. (Nature 2021; 598:137-43) (Brain Image Library: cf1c1a431ef8d021); Paired-seq co-assayed scRNA-seq and scATAC-seq from Zhu et al. (Nat Methods 2021; 18:283-92) (GEO: GSE152020).

### Code Availability.

The source code of the GAGE-seq data processing and analysis workflows can be accessed at: https://github.com/ma-compbio/GAGE-seq, which has also been deposited via Zenedo (https://doi.org/10.5281/zenodo.10888453) (Zhou T. GAGE-seq analysis workflow. 2024). In a GitHub repository, notebooks have been provided (https://github.com/ma-compbio/GAGE-seq/tree/main/scripts_analysis) that detail the integration between GAGE-seq and Paired-seq data for single-cell joint analysis of 3D genome structure, chromatin accessibility, and gene expression.

### Results

### Overview of GAGE-Seq.

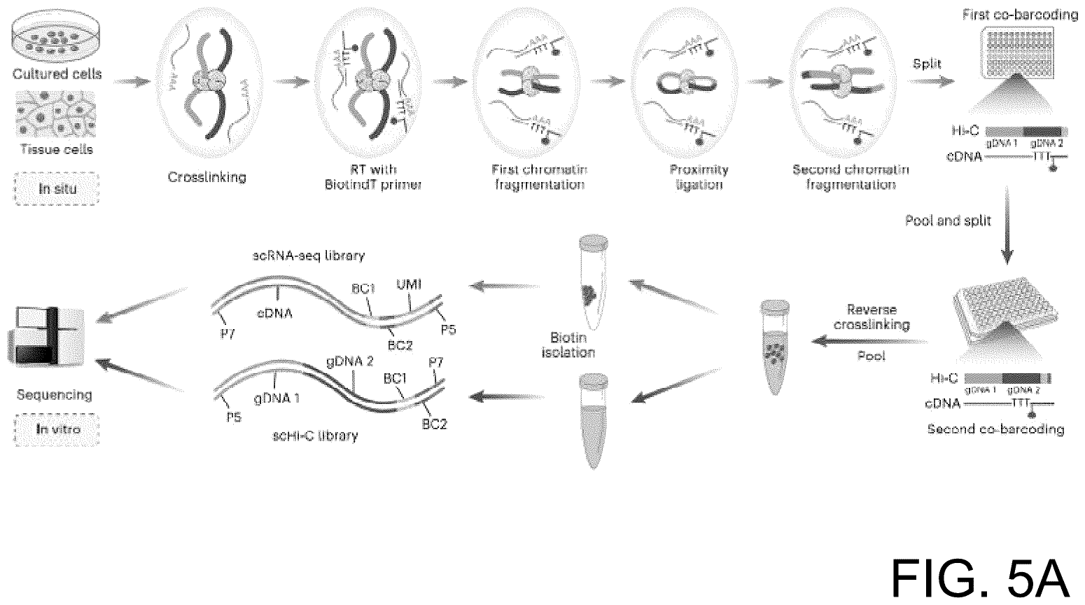

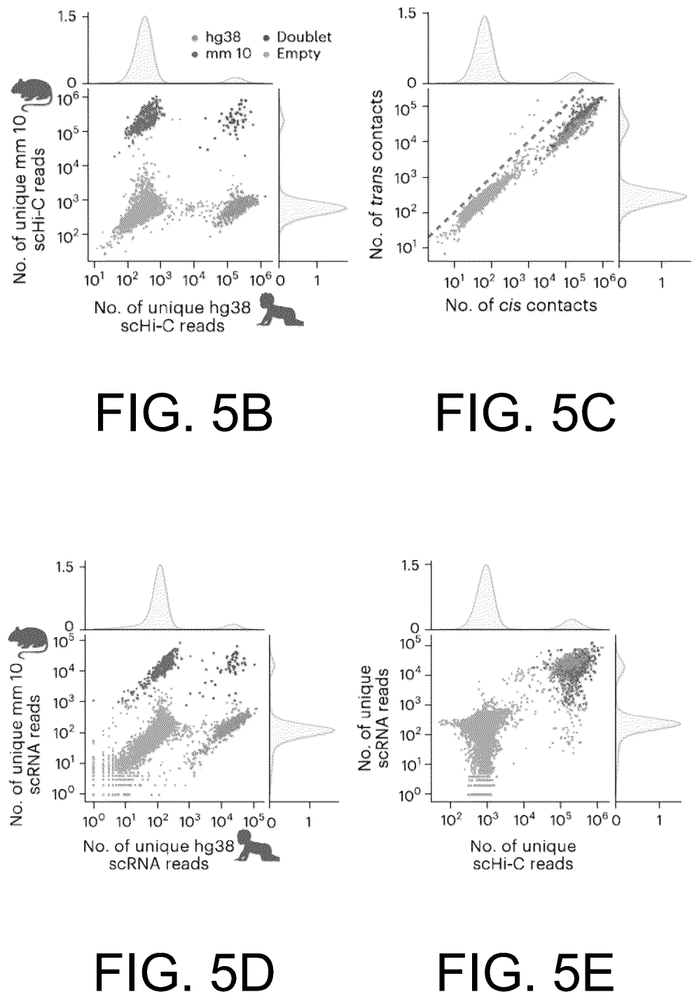

FIGS. 5A-5E illustrate an overview and validation of GAGE-seq. (5A) Schematic representation of the GAGE-seq workflow detailing the simultaneous single-cell profiling of 3D genome architecture and gene expression. (5B-5E) Validations demonstrating the specificity of GAGE-seq using mixed experiments with the human (K562) and mouse (NIH3T3). (5B. 5D) Scatter plots showing the collision level in the GAGE-seq scHi-C (5B) and scRNA-seq (5D) libraries, and histograms showing the binomial distribution of reads mapped to hg38 (top) and mm10 (right). (5C) Scatter plot showing the cis: trans ratio of scHi-C reads. (5E) Scatter plot showing the well-separation of scHi-C and scRNA reads of valid cellular indices from that of empty indices. Mouse is colored in green, human in orange, collisions in red, and empty indices in gray.

GAGE-seq is a high-throughput, effective, and robust single-cell multiomics technology that simultaneously profiles the 3D genome and transcriptome in individual cells (FIG. 5A). GAGE-seq leverages the highly scalable “combinatorial indexing” paradigm previously employed in sci-Hi-C(Ramani V, et al. Methods 2020; 170:61-8; Kim H-J, et al. PLOS Comput Biol 2020; 16; Bonora G, et al. Genome Biol 2021; 22:279), as well as other single-cell methods (Buenrostro J D, et al. Nature 2015; 523:486-90; Cusanovich D A, et al. Science 2015; 348:910-4; Cao J, et al. Science 2017; 357:661-7; Rosenberg A B, et al. Science 2018; 360:176-82) (FIG. 5A). The procedure can be summarized as follows: (i) The RNA in cross-linked and permeabilized cells or nuclei is reverse transcribed (RT) with a biotinylated poly (T) or random hexamer primer containing DNA sequences, facilitating the ligation of the first-round barcoded cDNA adaptors (FIGS. 4A-4D); (ii) Cross-linked chromatins are efficiently fragmented (the first round chromatin fragmentation) using two 4-cut restriction enzymes (RE), CviQI and Msel, both producing the same adhesive DNA end 5′-TA, enabling the identification of chromatin interactions via proximity ligation; (iii) After a second round of chromatin fragmentation to introduce adhesive DNA ends for ligating the first-round barcoded DNA adaptors (FIGS. 4A-4D), cells/nuclei are distributed to a 96-well plate, where the first-round barcodes for DNA or cDNA are introduced through ligation of barcoded adaptors; (iv) Intact cells/nuclei are then pooled, diluted, and redistributed to a second 96-well plate, where the second-round barcodes for DNA or cDNA are introduced through ligation; (v) After reverse-crosslinking to release barcoded nucleic acids, all genomic DNA and cDNA are pooled, and biotinylated cDNA fragments are separated from genomic DNA with streptavidin beads; (vi) Sequencing libraries for scHi-C and scRNA-seq are separately generated and sequenced (Methods); and finally, (vii) Matched scHi-C and scRNA-seq profiles are identified according to the well-specific barcoding combinations (FIGS. 5A, 4A-4D). This combinatorial cellular indexing strategy can be further extended to achieve even larger throughput using additional rounds of ligation-mediated barcoding. Quality validation and benchmarking of GAGE-seq.

To assess the quality and specificity of GAGE-seq data, experiments were performed using a mixture of human (K562) and mouse (NIH3T3) cell lines (FIGS. 5B-5E). Successful separation of human and mouse reads in both scHi-C and scRNA-seq data was demonstrated, identifying 683 human and 568 mouse cells out of 1,500 expected, along with 57 doublets observed in line with the expected 4.4% collision rate (FIGS. 5B-5E). Cells passing stringent quality criteria exhibited an average of 181,240 (K562, 39.2% duplicate rate) and 206,113 (NIH3T3, 38.0% duplicate rate) chromatin contacts (>1 Kb intra-chromosomal) for scHi-C, as well as an average of 24,784 (K562, 35.7% duplicate rate) and 16,596 (NIH3T3, 31.2% duplicate rate) unique molecular identifiers (UMIs) from 3,699 (K562) and 2,256 (NIH3T3) genes per cell for scRNA-seq (FIGS. 5A-5E). These robust results underscore GAGE-seq's ability to concurrently measure single-cell chromatin interactions and transcriptome with high sensitivity and accuracy. In addition, GAGE-seq's efficient fragmentation of crosslinked chromatin before proximity ligation, enabled by two four-cutters (FIG. 5A), allows for efficient detection of multi-way interactions, with >25% of all identified chromatin contacts in each scHi-C library.

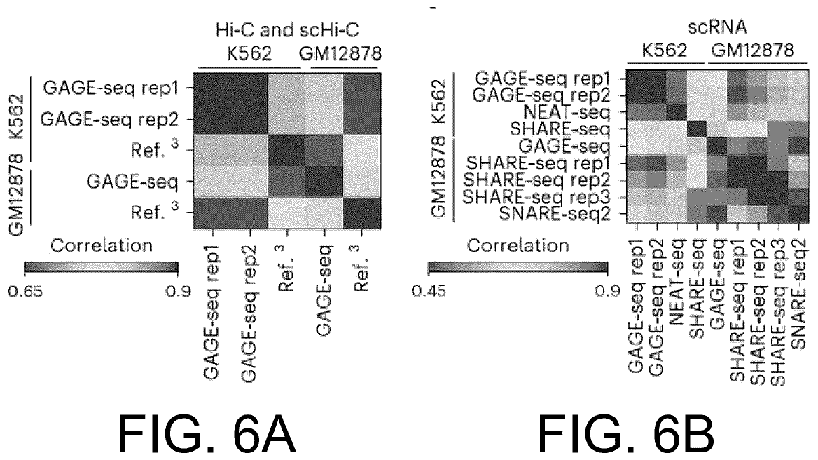

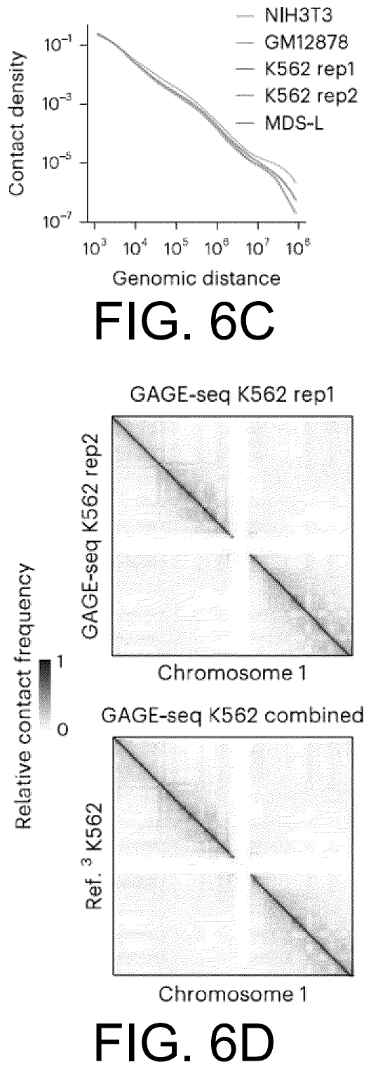

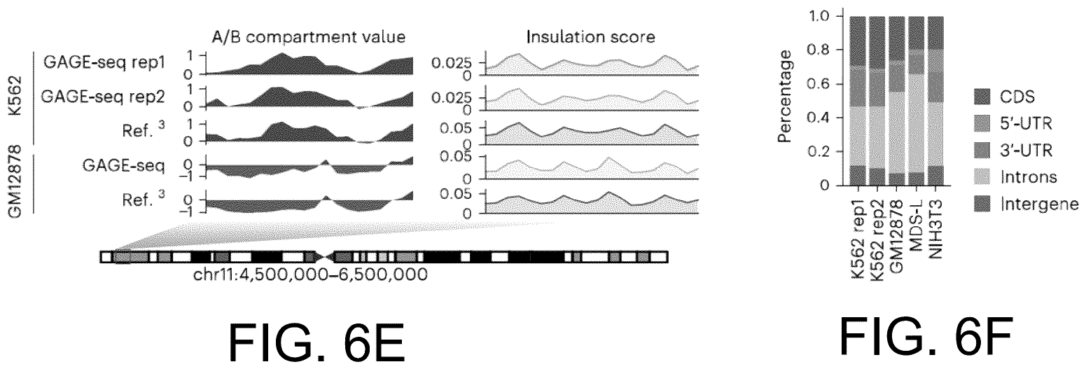

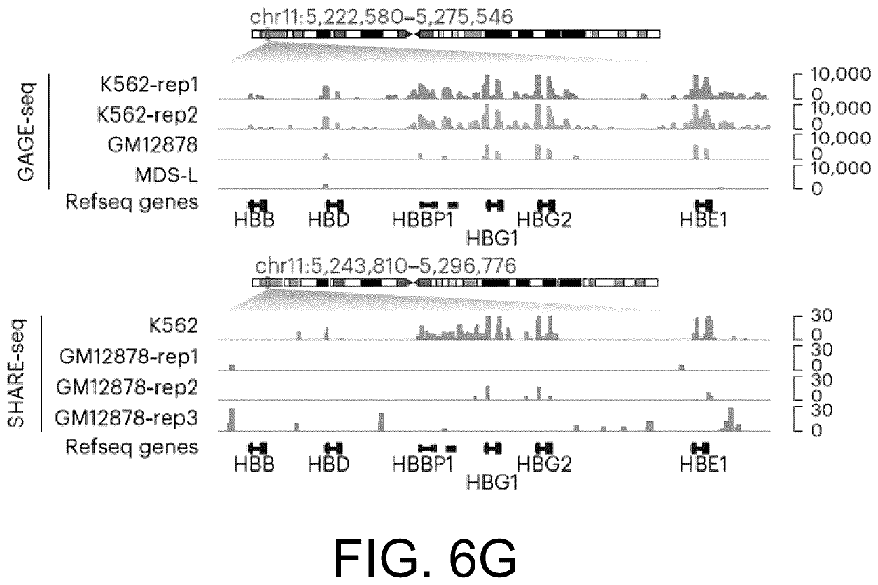

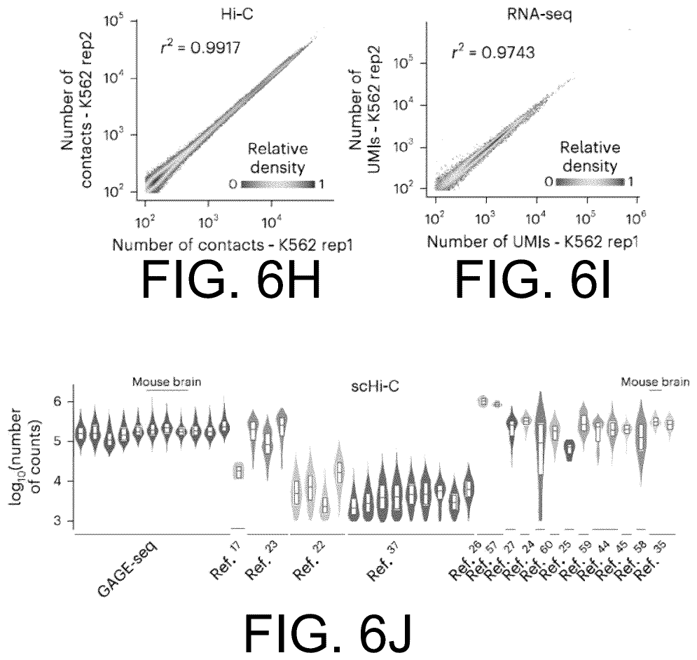

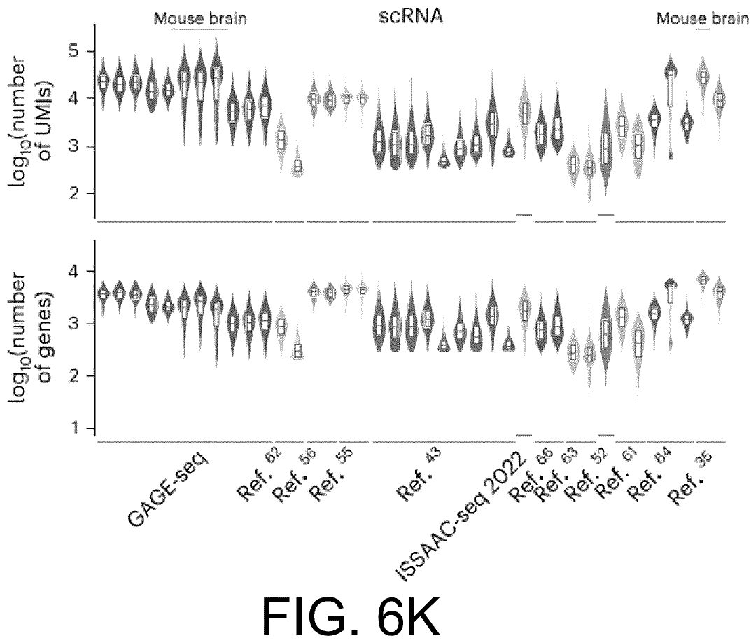

FIGS. 6A-6K illustrate high-quality scHi-C and scRNA-seq data generated by GAGE-seq. (6A) Pearson's correlation between the aggregated scHi-C profiles from GAGE-seq replicates and the bulk in situ Hi-C data (Rao S S P, et al. Cell 2014; 159:1665-80). (6B) Comparison of aggregated scRNA-seq profiles of GAGE-seq replicates with NEAT-seq (Chen A F, et al. Nat Methods 2022; 19:547-53), SHARE-seq (Ma S, et al. Cell 2020; 183:1103-1116), and SNARE-seq2 (Plongthongkum N, et al. Nat Protoc 2021; 16:4992-5029). Pearson's correlation is shown. (6C) Decay curves of chromatin contact for the GAGE-seq scHi-C libraries. (6D) Comparison of aggregated contact maps between two GAGE-seq K562 replicates (upper), and between the combined GAGE-seq K562 library and an in situ Hi-C library (Rao S S P, supra) (lower). (6E) Comparison of A/B compartments and TAD-like domain calling at the human beta-globin locus between GAGE-seq (pseudo bulk) and in situ Hi-C(Rao S S P, supra) (6F) RNA read distribution across gene bodies in the GAGE-seq scRNA libraries. (6G) Aggregated single-cell gene expression profiles at the GAPDH locus. Upper panel: scRNA-seq signals of GAGE-seq libraries of K562, GM12878, and MDS-L cells (hg38). Lower panel: scRNA-seq signals of SHARE-seq in GM12878 cells (hg19) (Ma S, supra). (5H) Reproducibility between two biological replicates of GAGE-seq scHi-C libraries. (5I) Reproducibility between two biological replicates of GAGE-seq scRNA libraries. r2 statistics are shown. (5J) Comparison of GAGE-seq scHi-C library size with published scHi-C(Nagano T, et al. Nature 2013; 502:59-64; Ramani V, et al. Nat Methods 2017; 14:263-6; Nagano T, et al. Nature 2017; 547:61-7; Flyamer I M, et al. Nature 2017; 544:110-4; Stevens T J, et al. Nature 2017; 544:59-64; Tan L, et al. Science 2018; 361:924-8; Tan L, et al. Cell 2021; 184:741-758; Kim H-J, et al. PLOS Comput Biol 2020; 16; Tan L, et al. Nat Struct Mol Biol 2019; 26:297-307; Mulqueen R M, et al. Nat Biotechnol 2021; 39:1574-80; Collombet S, et al. Nature 2020; 580:142-6; Gassler J, et al. EMBO J 2017; 36:3600-18) and co-assay methods (Liu Z, et al. Science 2023; 380:1070-6; Lee D-S, et al. Nat Methods 2019; 16:999-1006; Liu H, et al. Nature 2021; 598:120-8). (6K) Comparison of scRNA-seq library size (upper) and the number of detected genes (lower) with published co-assay methods (Liu Z, supra; Ma S, supra; Zhu C, et al. Nat Methods 2021; 18:283-92; Chen A F, et al. Nat Methods 2022; 19:547-53; Plongthongkum N, et al. Nat Protoc 2021; 16:4992-5029; Cao J, et al. Science 2018; 361:1380-5; Chen S, et al. Nat Biotechnol 2019; 37:1452-7; Zhu C, et al. Nat Struct Mol Biol 2019; 26:1063-70; Mimitou E P, et al. Nat Methods 2019; 16:409-12; Xu W, et al. Nat Methods 2022; 19:1243-9; Xiong H, et al. Nat Methods 2021; 18:652-60).

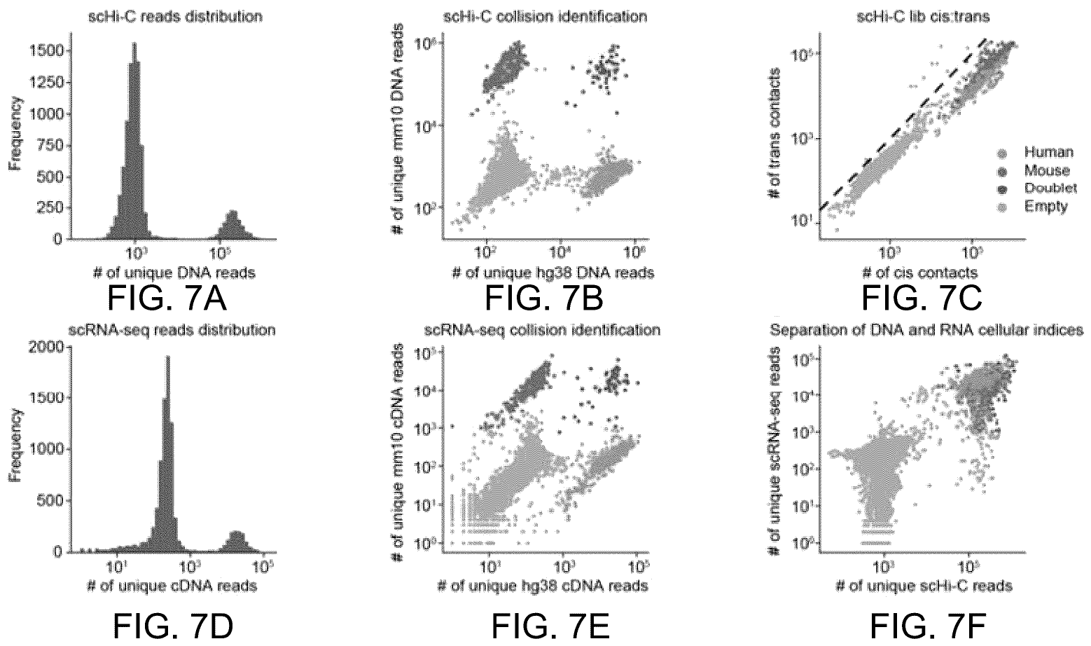

FIGS. 7A-7F illustrate quality-control assessment of the K562-GM12878 GAGE-seq libraries. (7A, 7D) Histogram showing the binomial distribution of GAGE-seq scHi-C (7A) and scRNA-seq reads (7D). (7B, 7E) Scatter plots representing the collision level in the GAGE-seq scHi-C (7B) and scRNA-seq (7E) libraries. (7C) Scatter plot showing the cis: trans ratio of scHi-C reads. (7F) Scatter plot indicating the clear separation of DNA and RNA reads of valid cellular indices from those of empty indices. Human data are colored in orange and empty indices in gray.

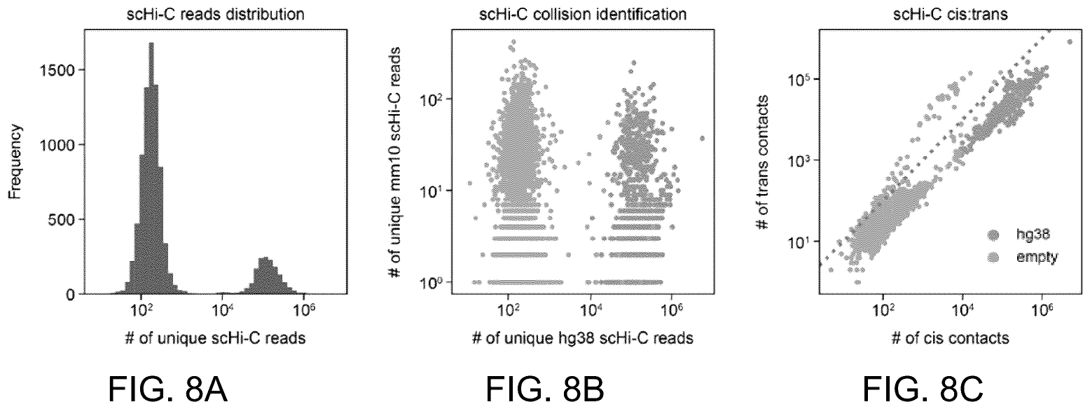

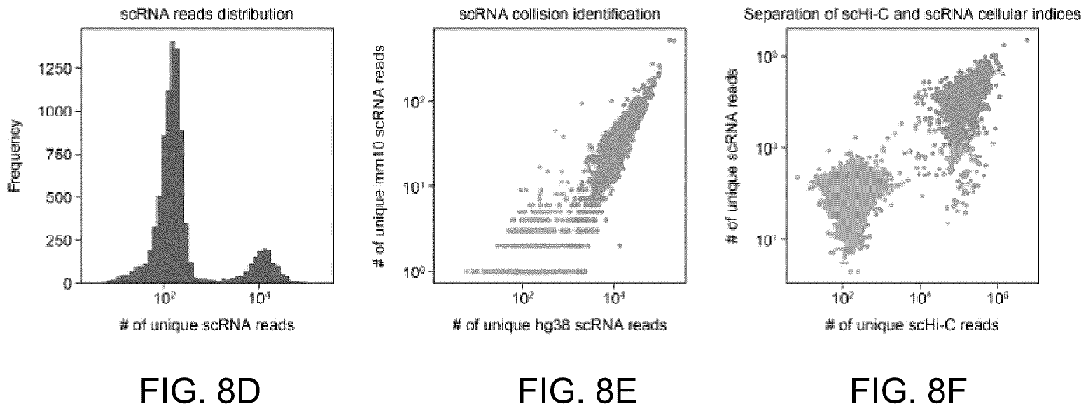

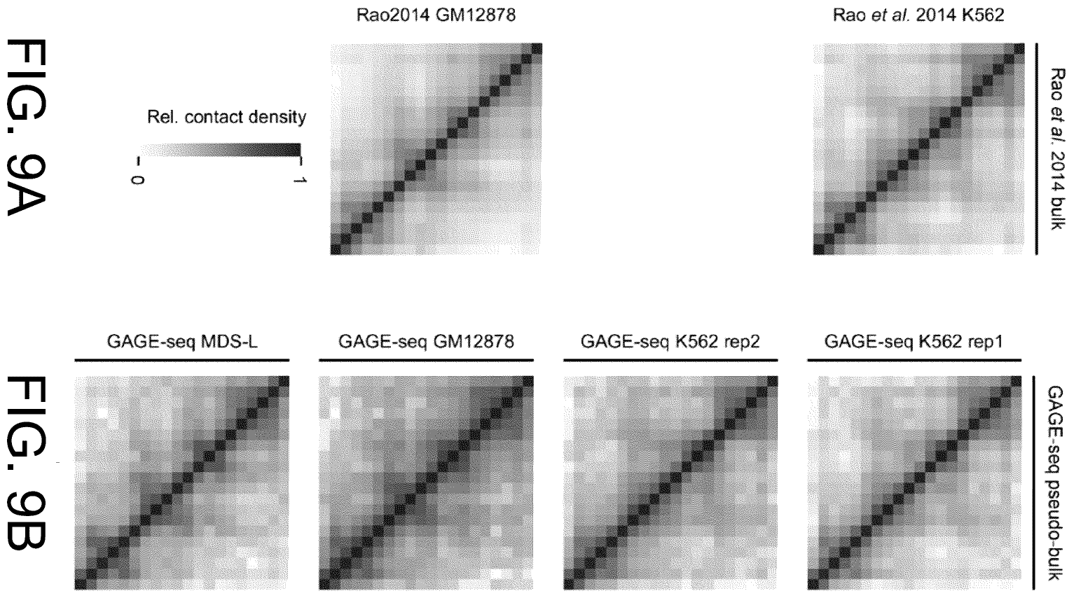

FIGS. 8A-8F illustrate quality-control assessment of the MDS-L GAGE-seq library. (8A, 8D) Histogram showing the binomial distribution of GAGE-seq scHi-C (8A) and scRNA reads (8D). (8B, 8E) Scatter plots showing the collision level in the GAGE-seq scHi-C (8B) and scRNA-seq (8E) libraries. (8C) Scatter plot showing the cis: trans ratio of scHi-C reads. (8F) Scatter plot indicating the clear separation of DNA and RNA reads of valid cellular indices from that of empty indices. Human data are colored in orange and empty indices in gray.

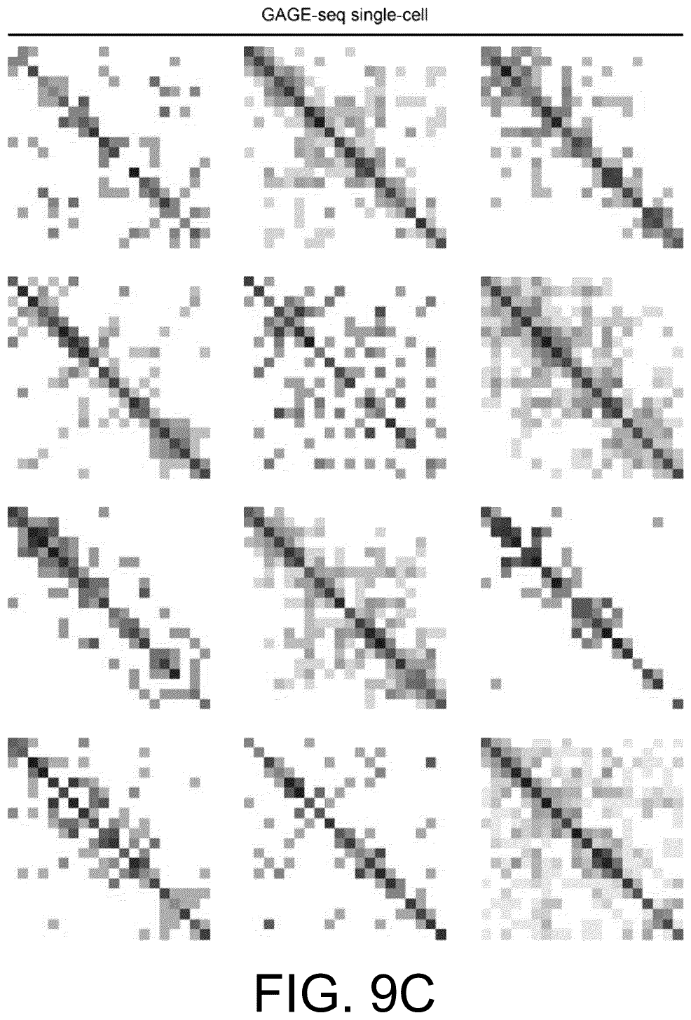

FIGS. 9A-9C illustrate single-cell and pseudo-bulk contact maps from the GAGE-seq datasets at the beta globin locus. (9A) Benchmarking contact maps from Rao et al (Cell 2014; 159:1665-80). (9B) GAGE-seq pseudo-bulk contact maps. (9C) Representative GAGE-seq single-cell contact maps. The displayed genomic location is human chr11: 4.5-6.5 Mb, and the resolution is 100 Kb.

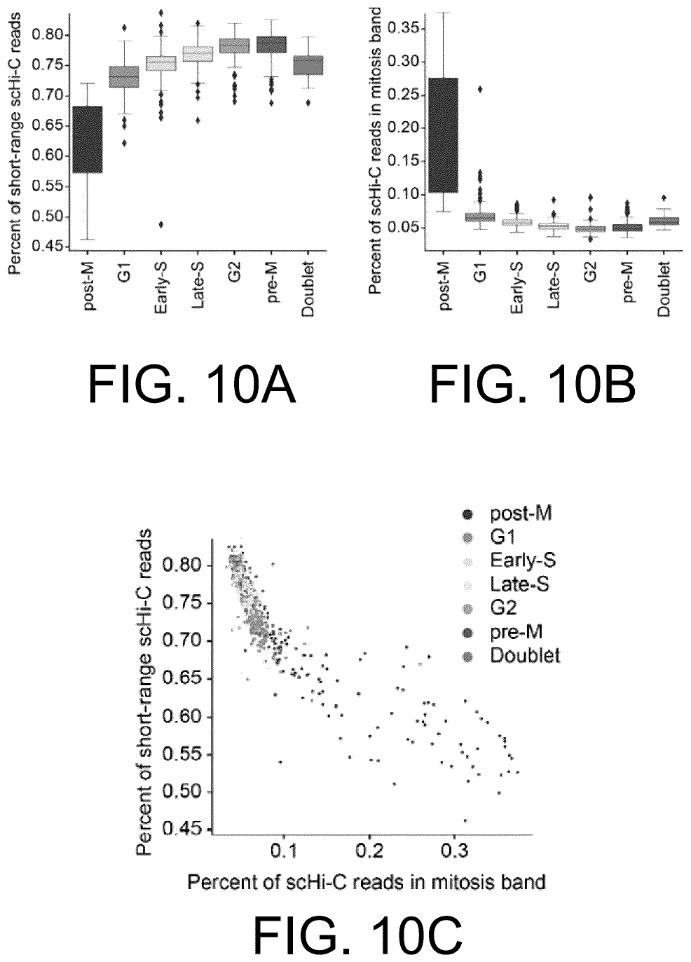

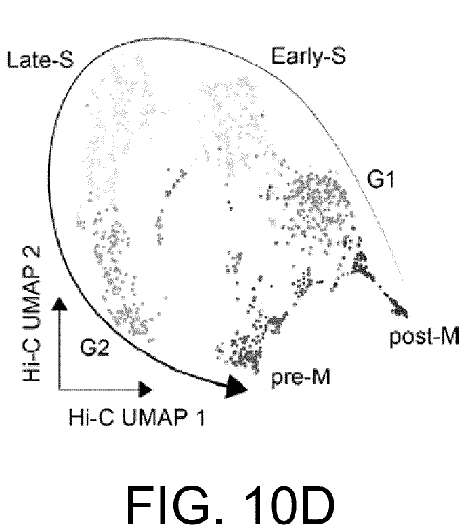

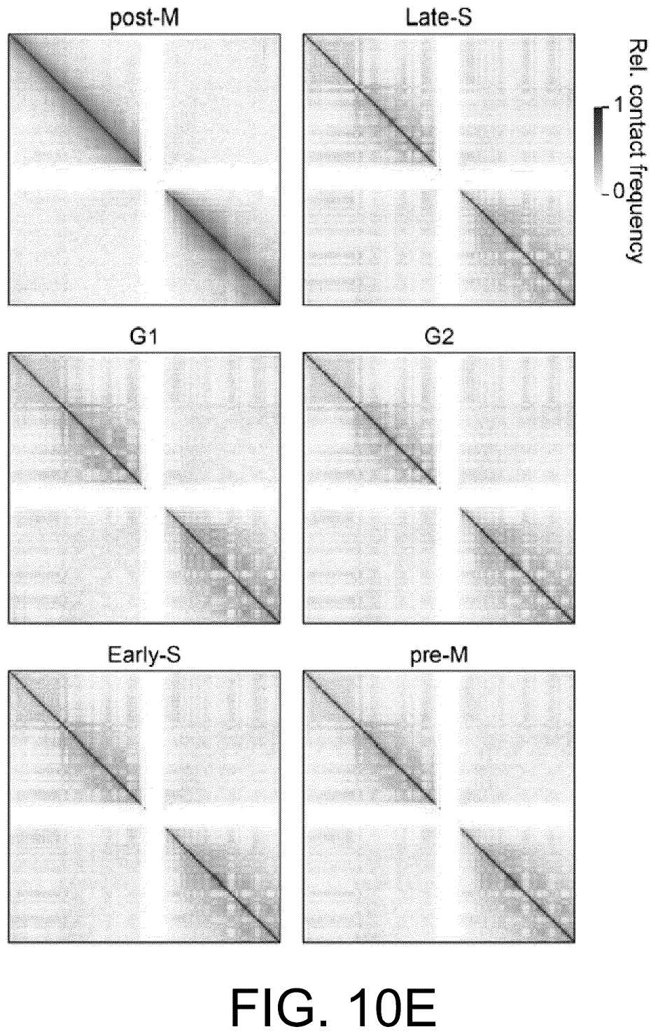

FIGS. 10A-10E illustrate cell cycle analysis of the GAGE-seq K562 cells. (10A) Percentage of short-range contacts (<2 Mb) in single cells. (10B) Percentage of mitosis-band contacts (2 to 12 Mb) in single cells. (10C) Joint visualization of the percentage of short-range and mitosis-band contacts in single cells. (10D) UMAP visualization of the Fast-Higashi embeddings. (10E) Aggregated contact maps of the 6 inferred cell cycle phases on chromosome 1 at 1 Mb resolution. All boxplots show the median, first, and third quartiles, and whiskers extend no further than 1.5× interquartile range.

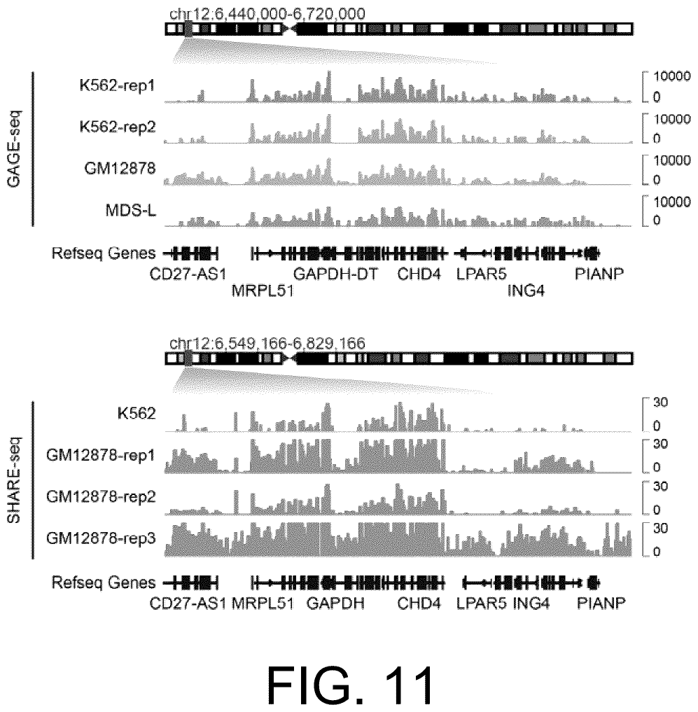

FIG. 11 illustrates aggregated single-cell gene expression profiles of the genes in the GAPDH locus. Upper panel: scRNA-seq signals from GAGE-seq libraries of K562, GM12878, and MDS-L cells (hg38). Lower panel: scRNA-seq signals from SHARE-seq in K562 and GM12878 cells (hg19) (Tan L, et al. Cell 2021; 184:741-758).

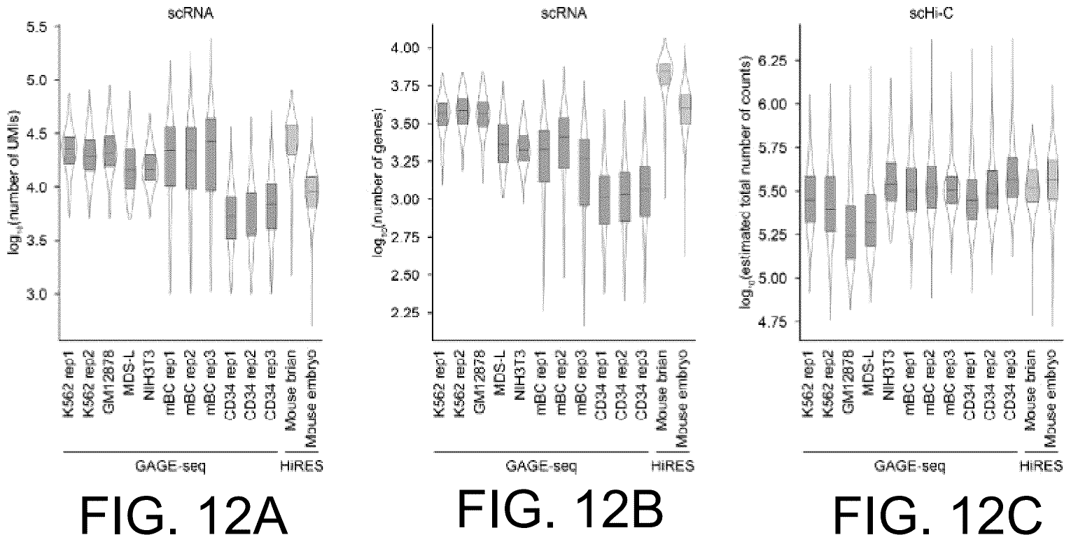

FIGS. 12A-12C illustrate a comparison of estimated scRNA and scHi-C library complexities between GAGEseq and HiRES28. (12A) The number of UMIs detected in single cells. (12B) The number of genes detected in single cells. (12C) The estimated number of chromatin contacts detected in the scHi-C libraries. All boxplots show the median, first, and third quartiles.

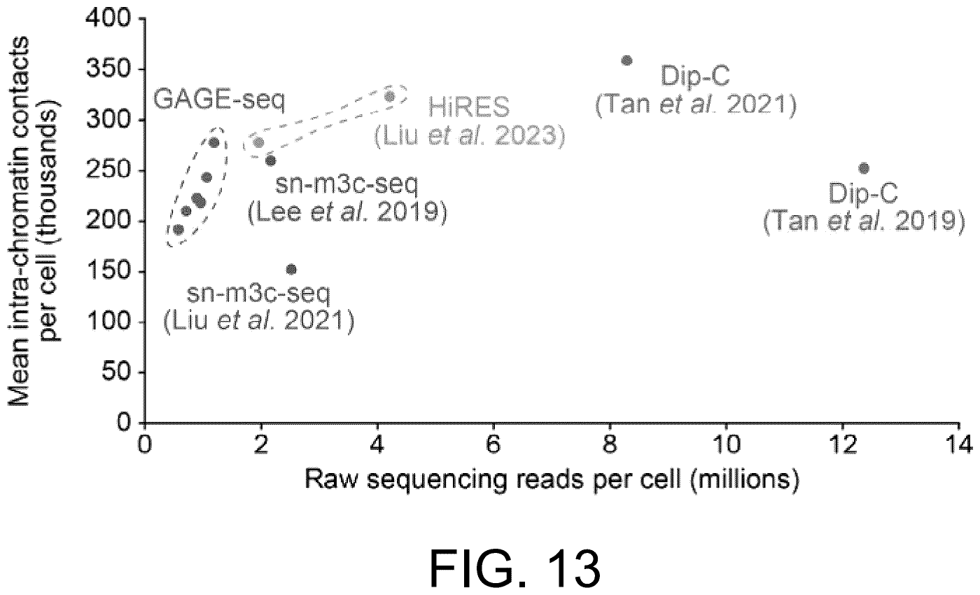

FIG. 13 illustrates a comparison between GAGE-seq and other scHi-C related methods in terms of efficiency. Intra-chromosomal chromatin contacts per single cell is shown against sequence depth between GAGE-seq scHi-C (blue), Dip-C (green) (Xiong K, Ma J. Nat Commun 2019; 10:5069; Dixon J R, et al. Nature 2012; 485:376-80) and sn-m3C-seq (red) (Zhang R, et al. Nat Biotechnol 2022; 40:254-61; Luo C, et al. Cell Genom 2022; 2 Luo C, et al. Cell Genom 2022; 2) and HiRES (orange) (Li G, et al. Nat Methods 2019; 16:991-3). The six blue dots represent the GAGE-seq libraries with mouse brain cortex and human CD34+ cells from this work.

Validating GAGE-seq in additional cell lines, GM12878 and MDS-L, further confirmed its robustness, specificity, sensitivity, and reproducibility (FIGS. 6A-6K, 7A-7F, 8A-8F). Whole-genome and whole-library level analysis showed GAGE-seq's chromatin interaction and gene expression profiles strongly correlating with published datasets (FIGS. 6A, 6B). Low collision rate (FIG. 5B), binomial distribution of scHi-C reads (FIGS. 5B, 7A, 8A), typical chromatin contact decay curve (FIG. 6C), high cis-trans ratio (FIGS. 50, 7C, 8C), and aggregated pseudobulk and single-cell chromatin contact maps (FIGS. 6D, 9A-9C, 10A-10E), as well as pseudobulk and single-cell A/B compartment scores and insulation scores (FIG. 6E), further confirmed the specificity of the GAGE-seq scHi-C signals. The specificity of the GAGE-seq scRNA-seq signals was demonstrated through low collision rate (4.6% in the K562/NIH3T3 library) (FIG. 5D), binomial distribution of RNA reads (FIGS. 5D, 7D, 8D), and the fact that the majority of RNA reads (86%) mapped to the gene body (FIG. 6F), complemented by the pseudobulk and single-cell RNA signal distribution at individual gene loci (FIGS. 6G, 11). Notably, similar to SHARE-seq43, GAGE-seq scRNA-seq reads were found to be 25%-50% intronic (FIG. 6F), indicating enriched nascent RNA. The high reproducibility across replicates was demonstrated at multiple levels (FIGS. 6A, B, D, E, G, H, I), and its methodological resolution (library complexity) of scHi-C matched existing lower-throughput, unimodal methods, such as Dip-C (Tan L, et al. Science 2018; 361:924-8; Tan L, et al. Cell 2021; 184:741-758), as well as sn-m3C-seq (Lee D-S, supra; Liu H, supra) (FIG. 6J). GAGE-seq scRNA-seq data quality was also comparable to existing methods (FIG. 6K). In line with previous scHi-C studies (Nagano, supra; Ramani V, et al. Methods 2020; 170:61-8), GAGE-seq scHi-C data revealed cell cycle stages (FIGS. 10A-10E). Compared to the recent HiRES method (Liu Z, supra), GAGE-seq offers major advantages in throughput, efficiency, and cost-effectiveness (FIGS. 6J, 6K, 12A-12C, 13), as well as in resolving rare cell types in complex tissues.

### GAGE-Seq Reveals Complex Cell Types in Mouse Cortex.

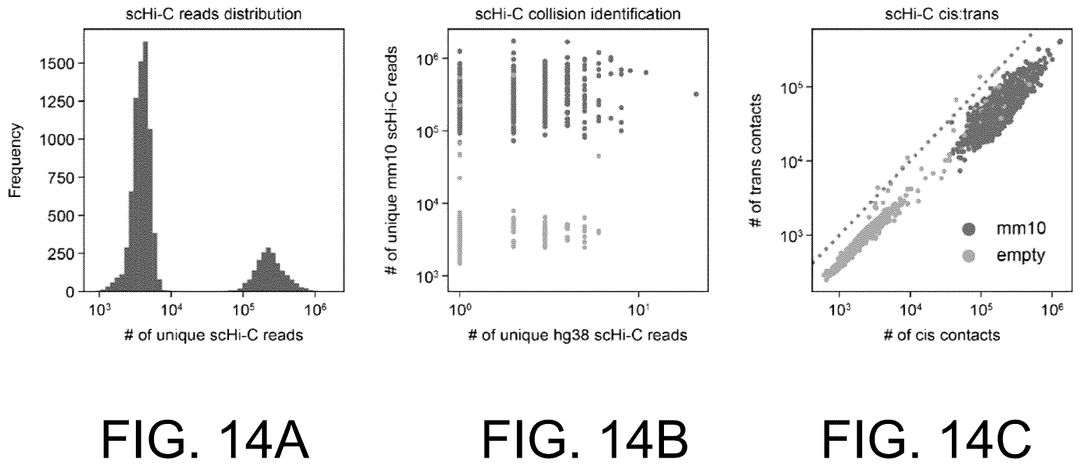

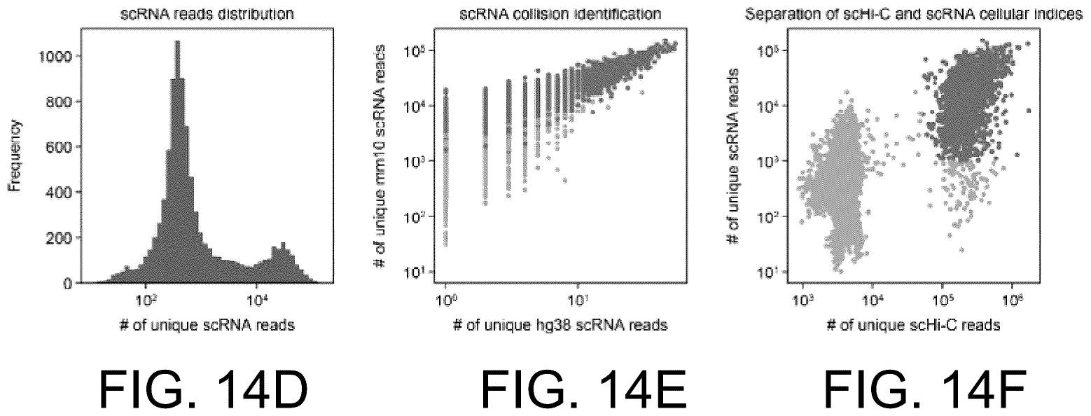

FIGS. 14A-14D illustrate a quality-control assessment of the GAGE-seq mouse brain cortex library (replicate 1). (14A, 14D) Histogram showing the binomial distribution of GAGE-seq scHi-C (14A) and scRNA-seq reads (14D). (14B, 14E) Scatter plots showing the collision level in the GAGE-seq scHi-C (14B) and scRNAseq (14E) libraries. (14C) Scatter plot showing the cis: trans ratio of scHi-C reads. (14F) Scatter plot showing the clear separation of DNA and RNA reads of valid cellular indices from that of empty indices. Mouse data are colored in green and empty indices in gray.

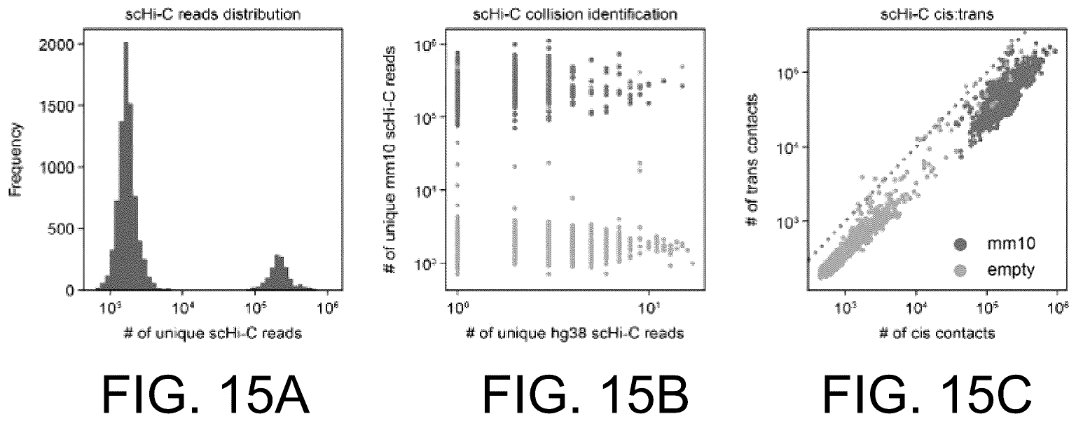

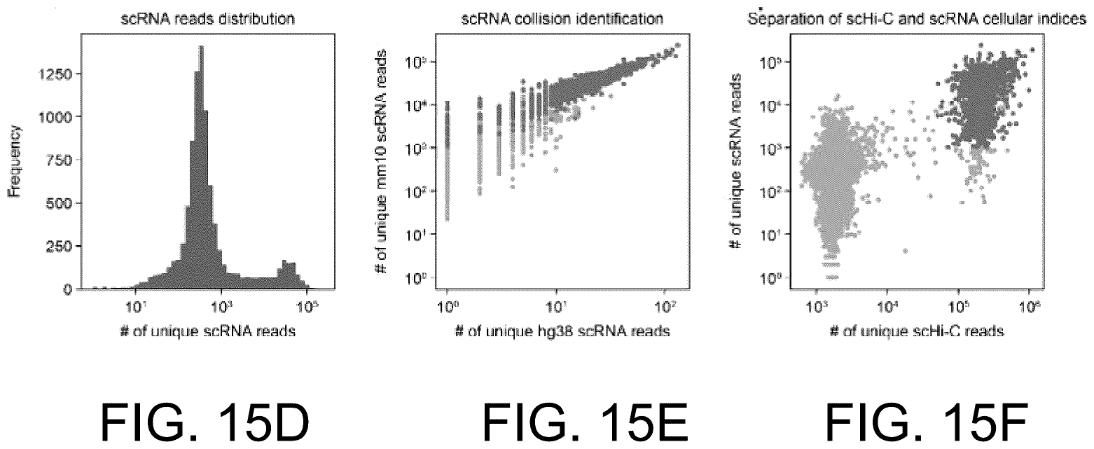

FIGS. 15A-15F illustrate a quality-control assessment of the GAGE-seq mouse brain cortex library (replicate 3). (15A, 15D) Histogram showing the binomial distribution of GAGE-seq scHi-C (15A) and scRNA-seq reads (15D). (15B, 15E) Scatter plots showing the collision level in the GAGE-seq scHi-C (15B) and scRNAseq (15E) libraries. (15C) Scatter plot showing the cis: trans ratio of scHi-C reads. (15F) Scatter plot showing the well-separation of DNA and RNA reads of valid cellular indices from that of empty indices. Mouse data are colored in green and empty indices in gray.

To demonstrate the utility of GAGE-seq in unveiling complex cell types based on single-cell 3D genome features and gene expression within a tissue context, the focus was turned to the adult mouse brain cortex, known for its cell type diversity. Applying GAGE-seq on cells from the mouse cortex (8-9 weeks old), 3,296 high-quality joint single-cell profiles of chromatin interactions and transcriptomes were generated. On average, each cell displayed 231,136 chromatin contacts (at 50% duplication rate), with 20,160 UMIs and 1,883 genes per cell (59% duplication rate), in line with the adult mouse whole brain data from the recently published HiRES data (FIGS. 14A-14D, 15A-15D, 6J, 6k).

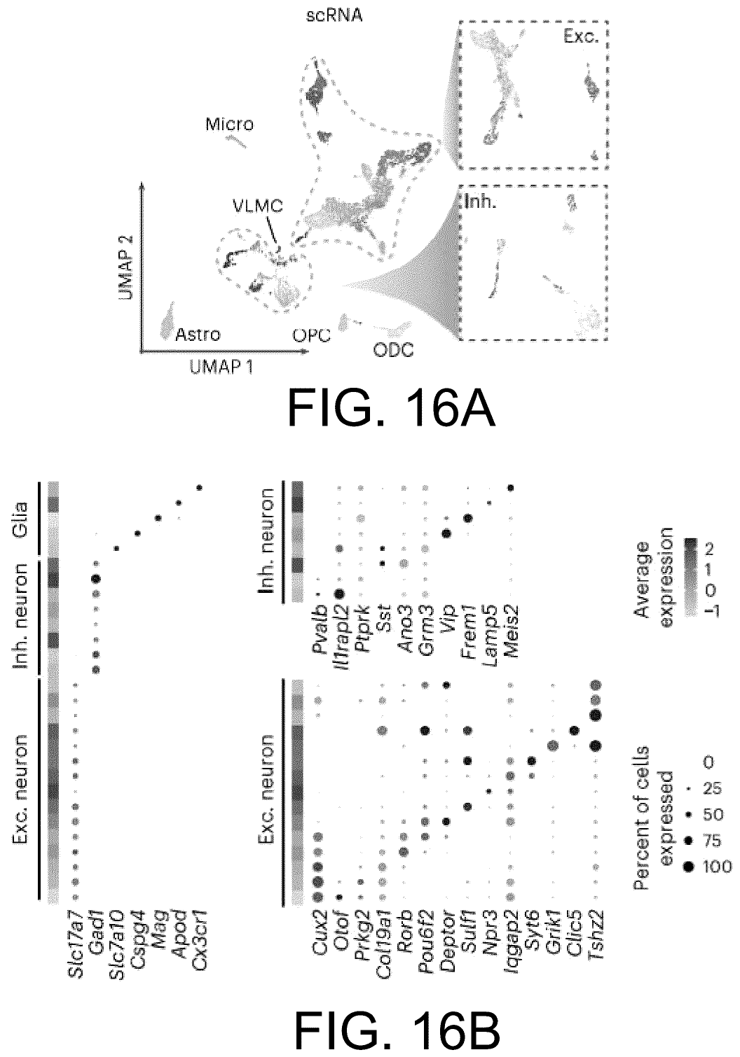

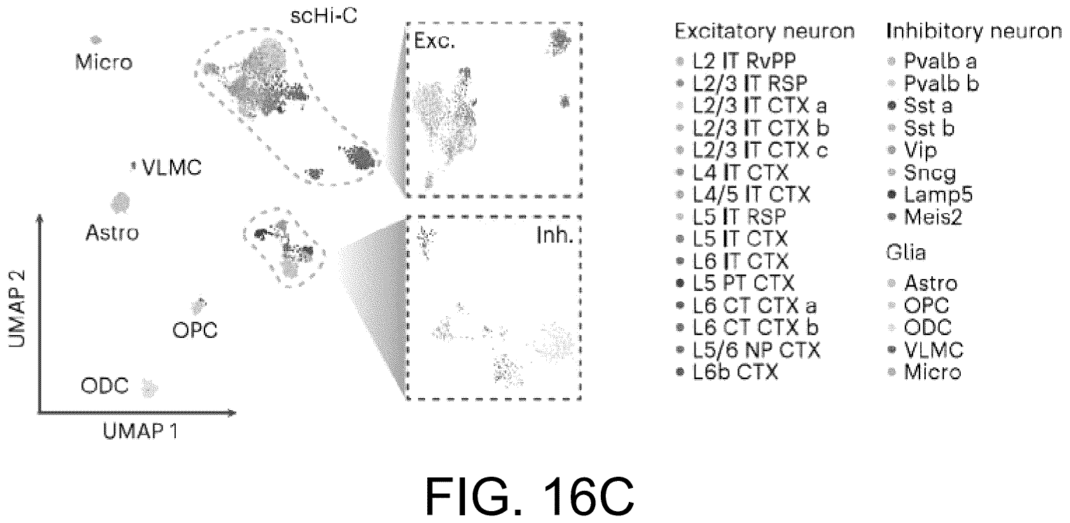

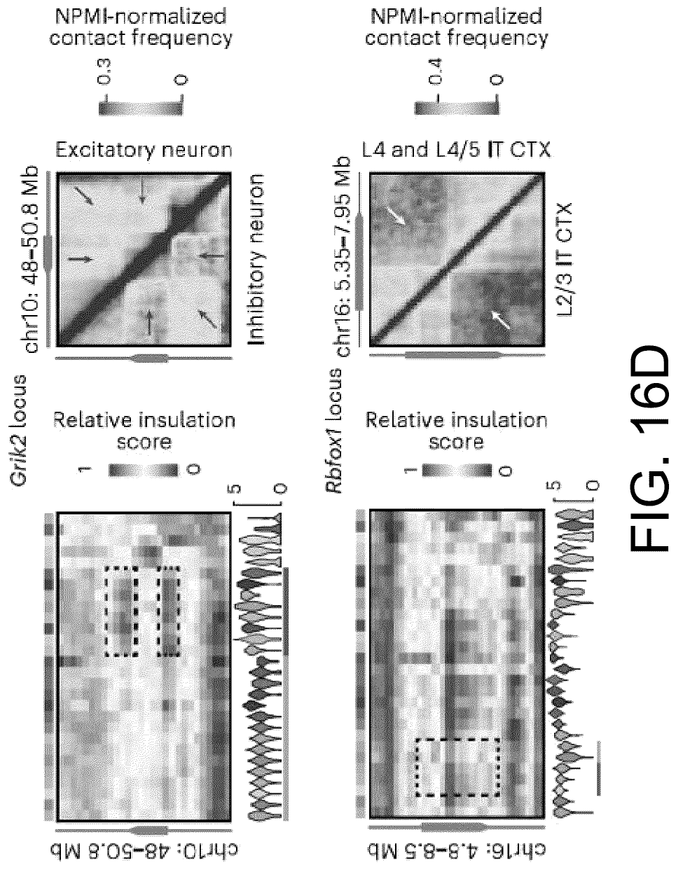

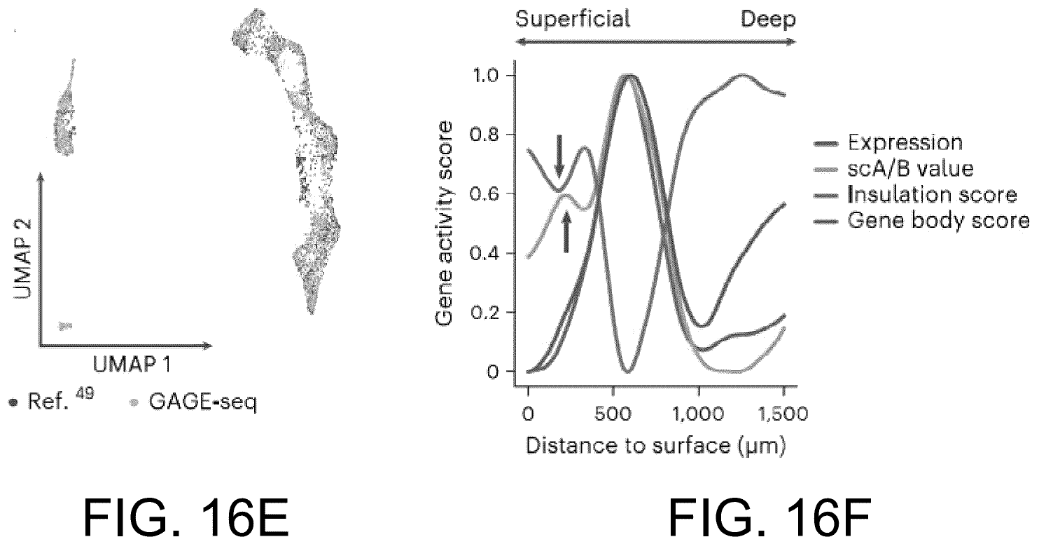

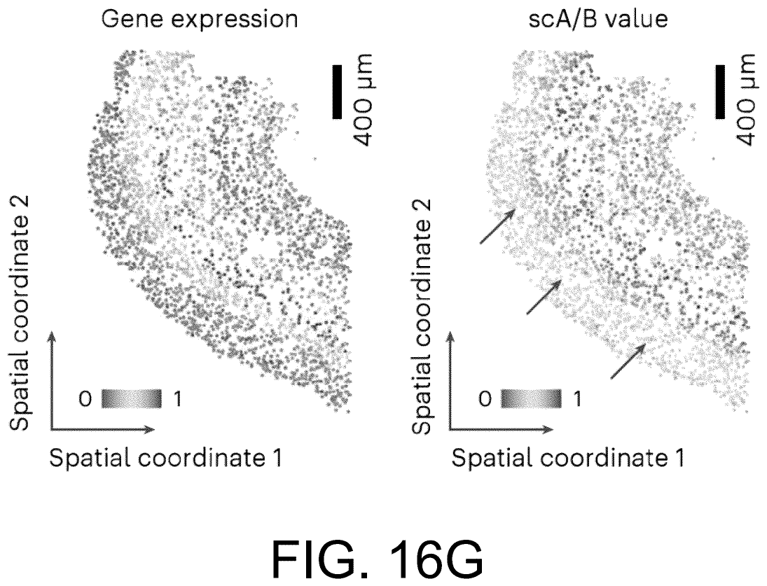

FIGS. 16A-16G illustrate cell types in mouse cortex characterized by GAGE-seq scHi-C and scRNA-seq. (16A, 16C). UMAP visualization of mouse cortex scRNA-seq (16A) and scHi-C profiles (16C) from GAGE-seq. Insets: UMAP visualization of excitatory neuron subtypes (top) and inhibitory neuron subtypes (bottom). (16B) Cell type-specific expression (based on scRNA-seq in GAGE-seq) of known marker genes, including glial types, neuronal types, and neuron subtypes. (16D) Visualization of cell type-specific 3D chromatin architecture and gene expression at representative gene loci. Left: aggregated single-cell insulation score (100-Kb resolution, upper) and gene expression (lower) at the Girk2 locus and the Rbfox1 locus. Right: aggregated contact maps (50-Kb resolution) of the Girk2 locus (top panel, excitatory vs inhibitory neurons) and the Rbfox1 locus (low panel, L4 & L4/5 IT CTX vs L2/3 CTX). Cell types selected in the right panels are highlighted by green lines (higher expression) or red lines (lower expression) in the corresponding left panels. (16E) UMAP visualization of the integration of GAGE-seq and a MERFISH dataset (Zhang M, et al. Nature 2021; 598:137-43). (16F) Inferred spatial patterns of gene expression and 3D genome features of L5 IT CTX marker genes. (16G) In situ plots of inferred single-cell gene expression (left) and scA/B value (right) for L5 IT CTX marker genes. Layer 3 was highlighted by black arrows in panels (16F) and (16G). The cell type abbreviations are based on the naming convention used in (Yao Z, et al. Cell 2021; 184:3222-3241).

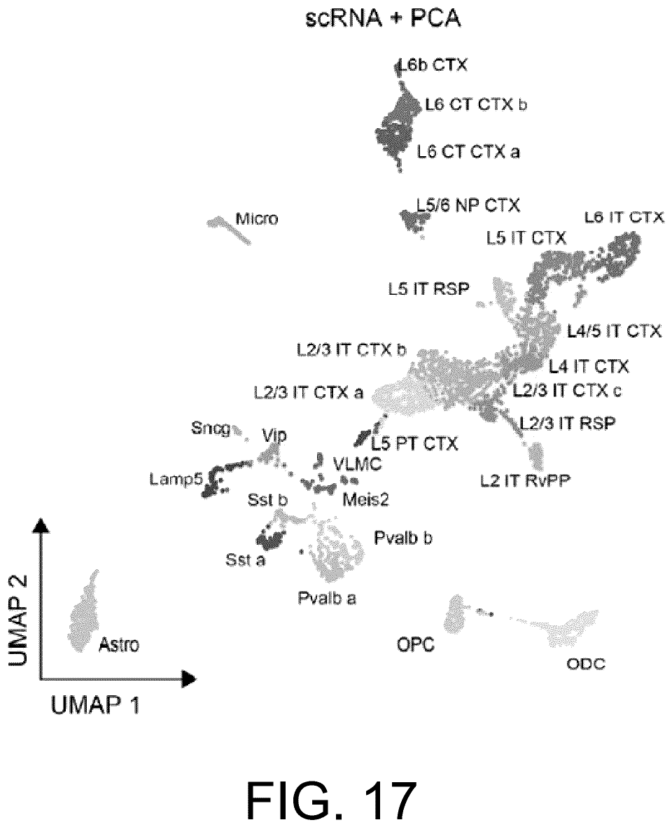

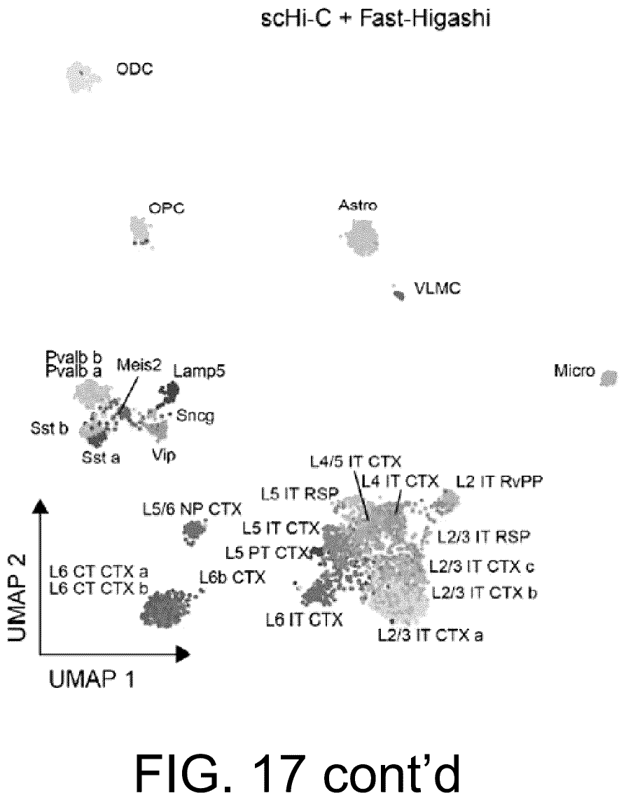

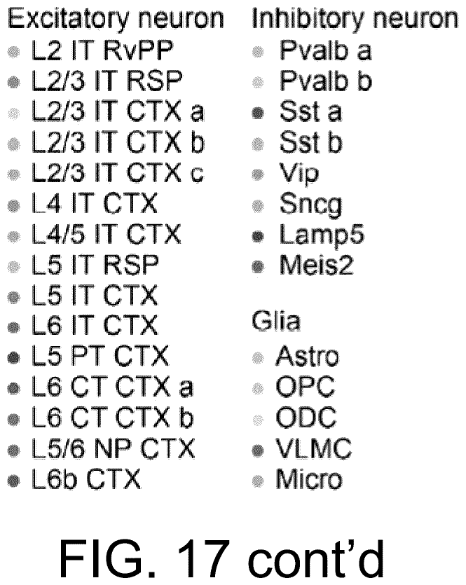

FIG. 17 illustrates high resolution cell type identification in the mouse brain cortex using GAGE-seq. First panel: UMAP visualization of the PCA embeddings of GAGE-seq scRNA-seq profiles. Second panel, UMAP visualization of the Fast-Higashi embeddings of GAGE-seq sHi-C profiles. A strong correlation between the “structure” (scHi-C-based) and transcriptome (scRNA-based) cell types is observed.

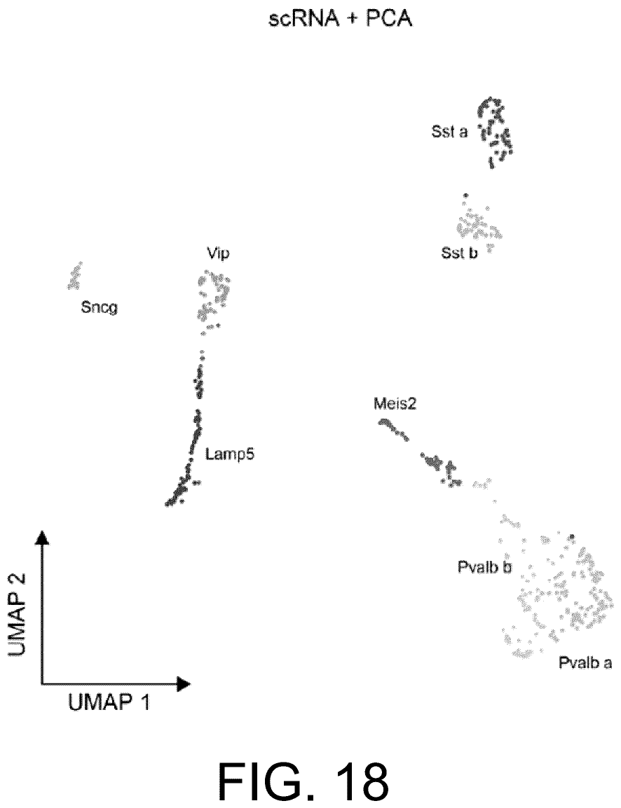

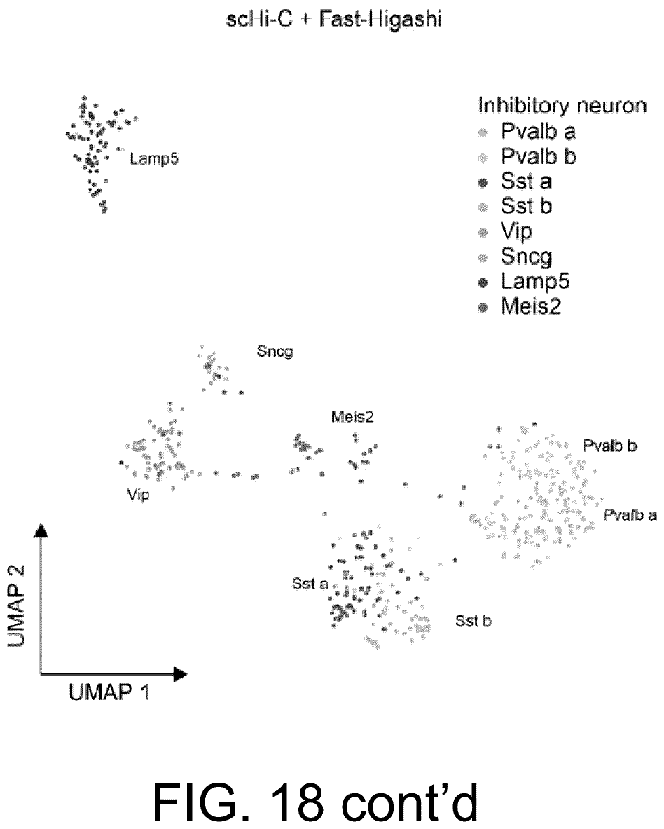

FIG. 18 illustrates high resolution inhibitory neuron subtypes revealed by GAGE-seq. First panel, UMAP visualization of the PCA embeddings of GAGE-seq scRNA-seq profiles. Second panel, UMAP visualization of the Fast-Higashi embeddings of GAGE-seq sHi-C profiles. A strong correlation between the “structure” (scHi-C-based) and transcriptome (scRNA-based) cell types is observed.

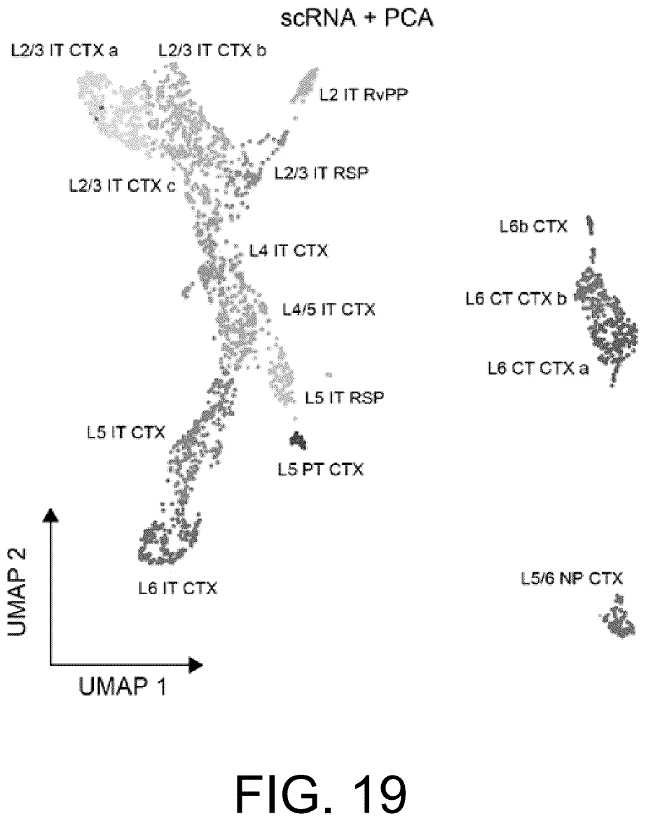

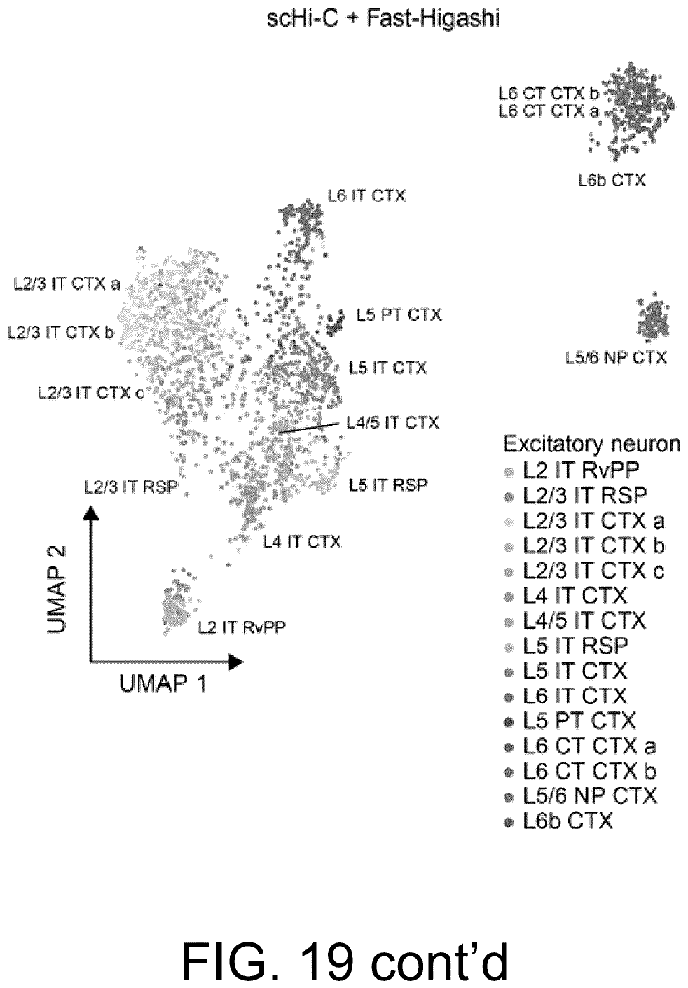

FIG. 19 illustrates high resolution excitatory neuron subtypes revealed by GAGE-seq. First panel, UMAP visualization of the PCA embeddings of GAGE-seq scRNA-seq profiles. Second panel, UMAP visualization of the Fast-Higashi embeddings of GAGE-seq sHi-C profiles. A strong correlation between the “structure” (scHi-C-based) and transcriptome (scRNA-based) cell types is observed.

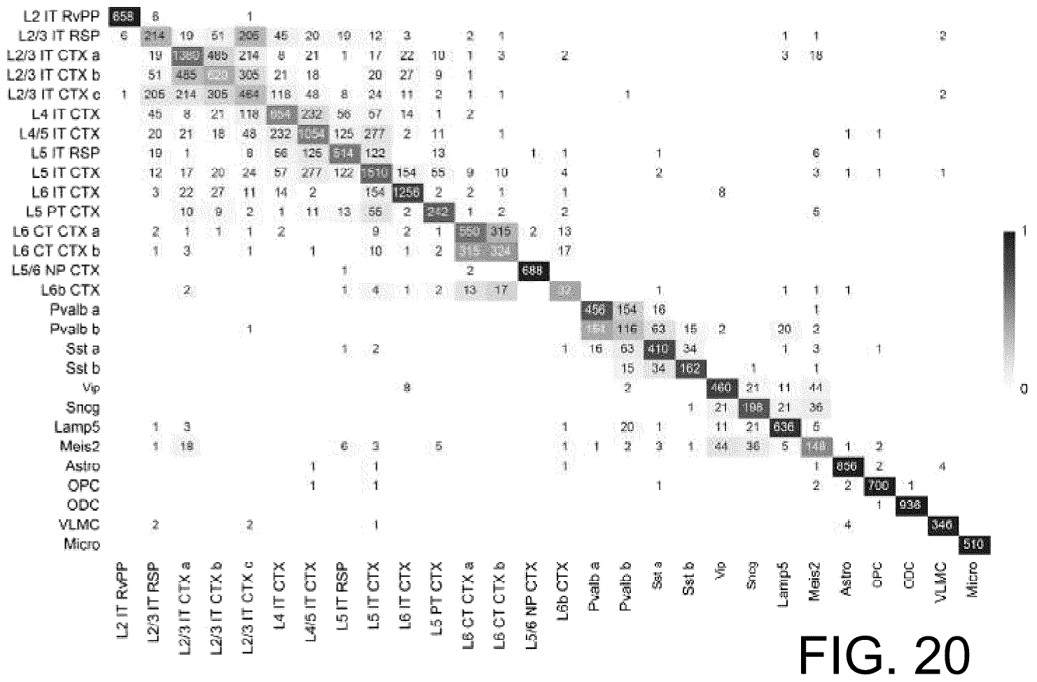

FIG. 20 illustrates congruence between GAGE-seq scRNA-seq clusters and scHi-C embeddings. The presented adjacency matrix shows the mutual 20-nearest neighbor graph of the Fast-Higashi embeddings, aggregated by cell types. Color intensity is normalized per row. When comparing the pairwise adjacency and self-adjacency, Fisher's exact test was applied to the 2-by-2 adjacency matrix for each pair of cell types. The maximum P-value of the one-sided Fisher's exact test was 2e-7, indicating all 28 cell types can be separated in the Fast-Higashi embeddings.

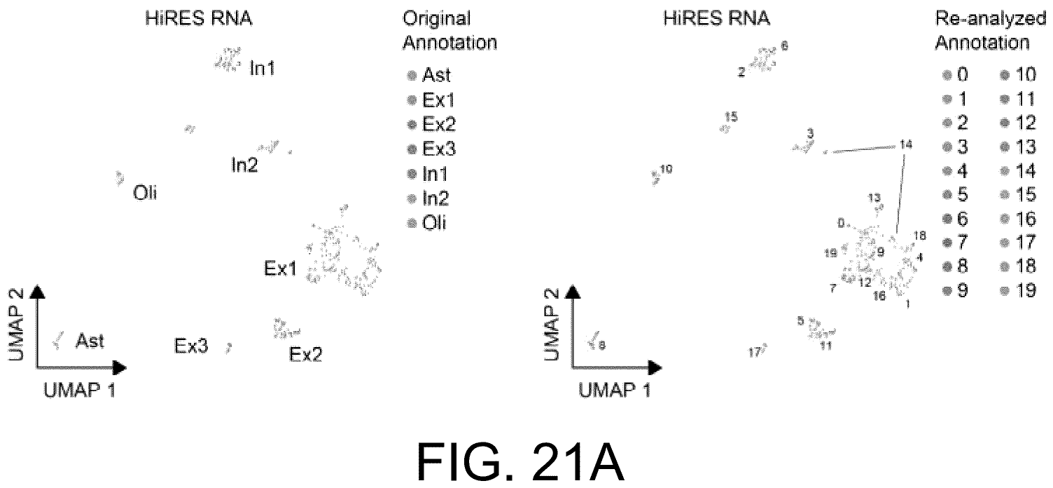

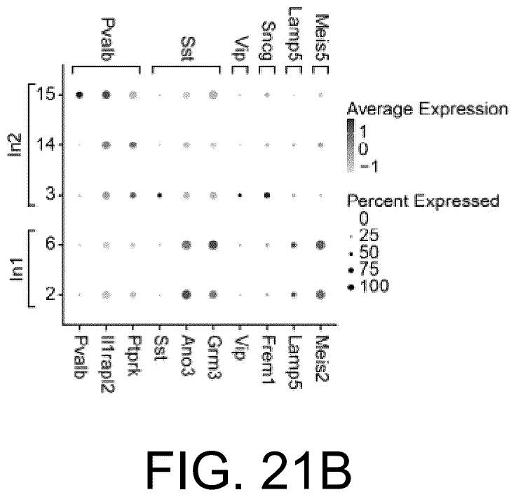

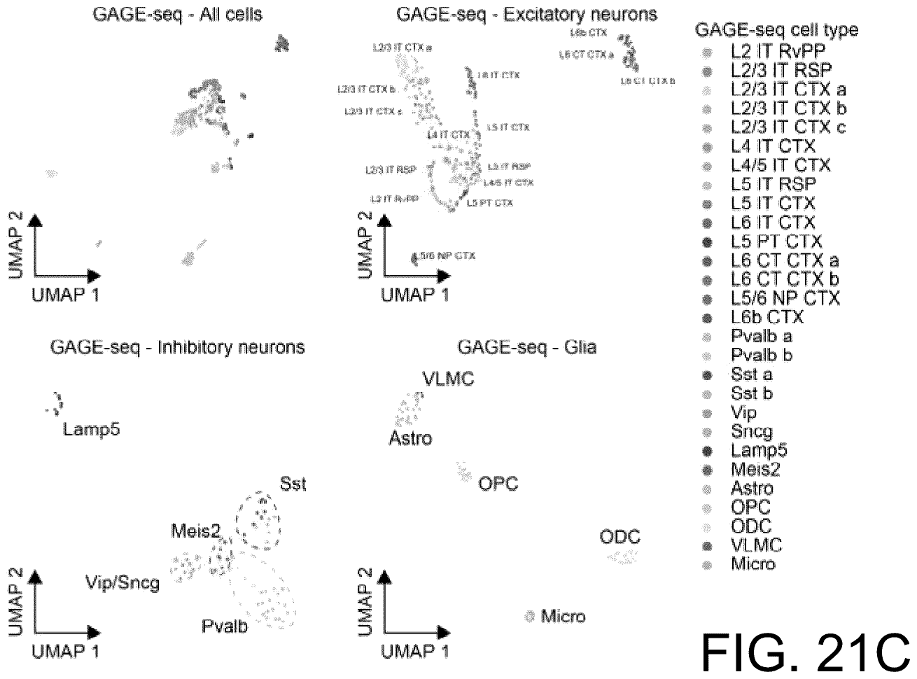

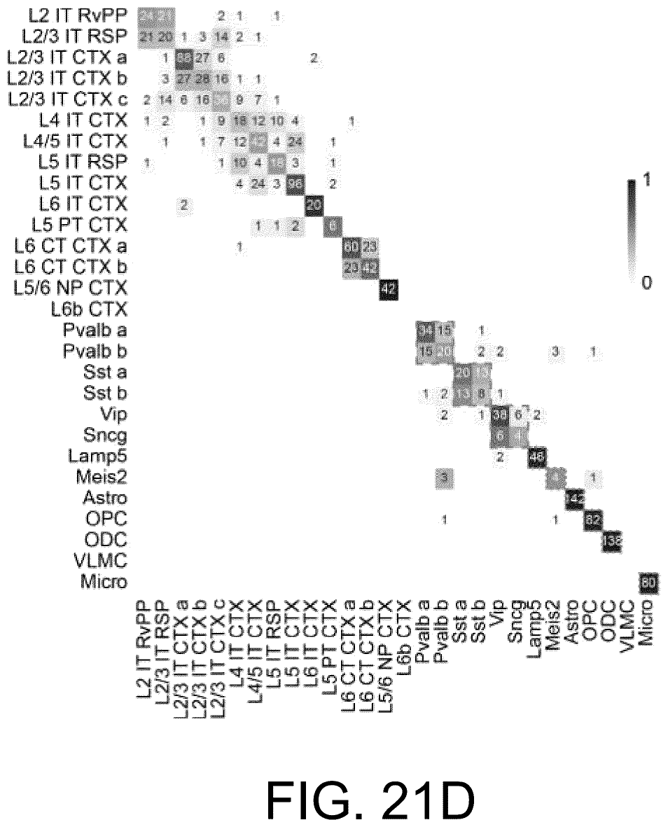

FIGS. 21A-21D illustrate re-analysis of scRNA profiles of HiRES and downsampled GAGE-seq from the mouse brain. (21A) UMAP visualization of HiRES scRNA embeddings generated by the described pipeline. Cells are colored by original cell type annotations (left) and clusters inferred by the pipeline (right), respectively. (21B) Expressions of inhibitory marker genes in the 5 finer inhibitory clusters inferred by the pipeline. (21C) UMAP visualization of downsampled GAGE-seq scRNA embeddings, colored by the original cell type annotations. (21D) The adjacency matrix of the mutual k-Nearest Neighbor graph of the downsampled GAGE-seq scRNA embeddings. Green rectangles highlight 5 inhibitory subtypes and 4 glial types that can be identified from the downsampled GAGE-seq data.

The disclosed GAGE-seq scRNA-seq data identified 28 known cell types across three major lineages in the mouse cortex, including 15 excitatory neuron subtypes, 8 inhibitory neuron subtypes, and 5 glial cell subtypes, such as astrocytes and oligodendrocytes (FIGS. 16A, 16B, 17, 18). These cell identities were confirmed by unique marker gene expressions (FIG. 16B). Notably, GAGE-seq scRNA-seq data enabled the delineation of many rare neuronal subtypes not identified by HiRES35, such as L5 PT CTX, Sncg, and Meis2 (FIGS. 16A, 16B, 18-20). Reanalysis of HiRES mouse brain data with Fast-Higashi (Zhang R, et al. Cell Syst 2022; 13:798-807) further confirmed the superior performance of GAGE-seq in identifying complex cell subtypes, despite a lower sequencing depth in GAGE-seq (FIGS. 21A-21D). Although 3D genome features are known to encode cell identity (Liu Z, supra; Winick-Ng W, et al. Nature 2021; 599:684-91), scHi-C often identified fewer cell types in complex tissues than scRNA-seq (Tan L, et al. Cell, supra; Lee D-S supra; Liu H, supra; Heffel M G, et al. BioRxiv 2022:2022.10.07.511350). Utilizing Fast-Higashi for scHi-C embedding, GAGE-seq distinguished all 28 transcriptome-defined cell types, including the aforementioned L5 PT CTX, Sncg, and Meis2 rare subtypes (FIGS. 16C, 18-20). The scHi-C-based delineation supports these cell types with distinct 3D genome features, with insulation scores surrounding gene bodies showing cell type-specific connection with gene expression (FIG. 16D).

### Spatial Integration Reveals In Situ 3D Genome Variation.

Using GAGE-seq to map the 3D genome and transcriptome of single cells, the in situ variation of the 3D genome in the adult mouse cortex was explored. GAGE-seq scRNA-seq was leveraged as a “bridge” for this analysis. Recently, the spatial transcriptomics method MERFISH successfully discerned the spatial organization of distinct cell populations in the mouse primary motor cortex (Zhang M, supra). This was started by integrating the disclosed GAGE-seq scRNA-seq data with the MERFISH data using Seurat (Chidester B, et al. Nat Genet 2023; 55:78-88), enabling the establishment of a connection between the two datasets.

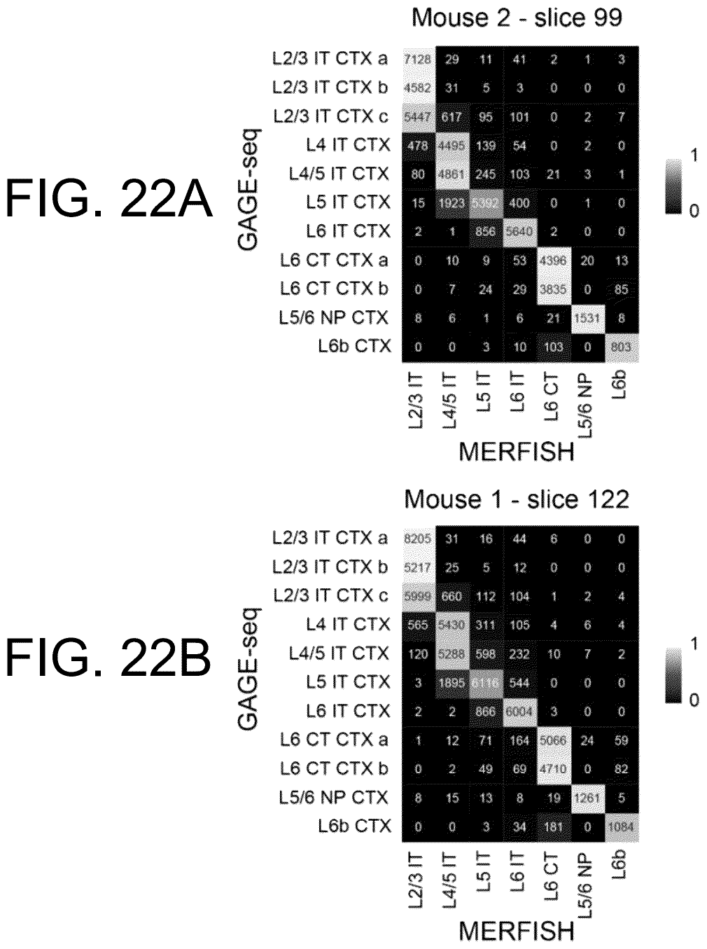

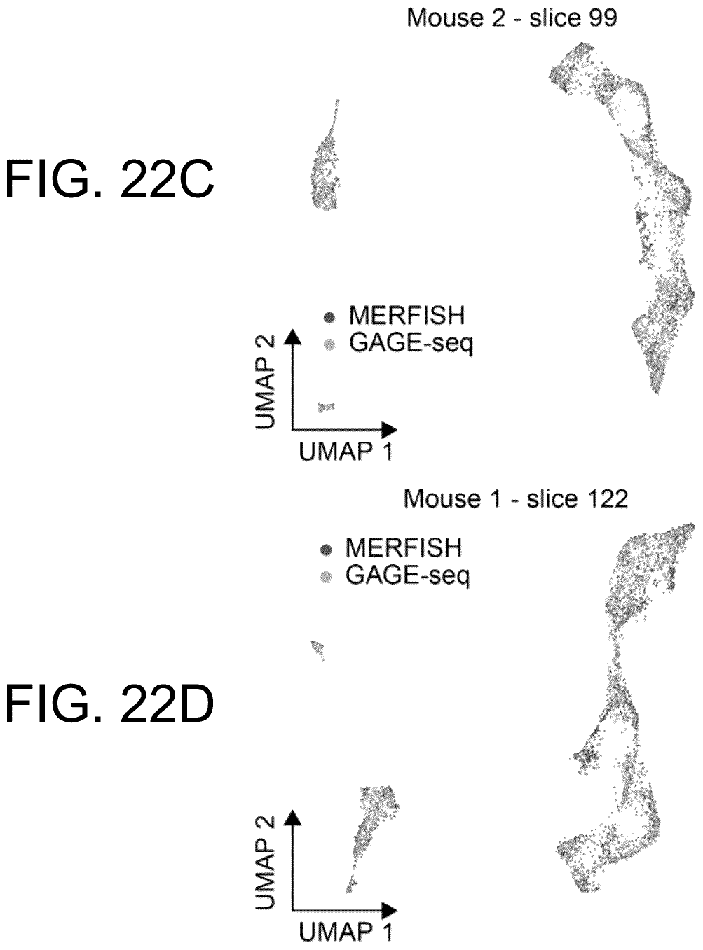

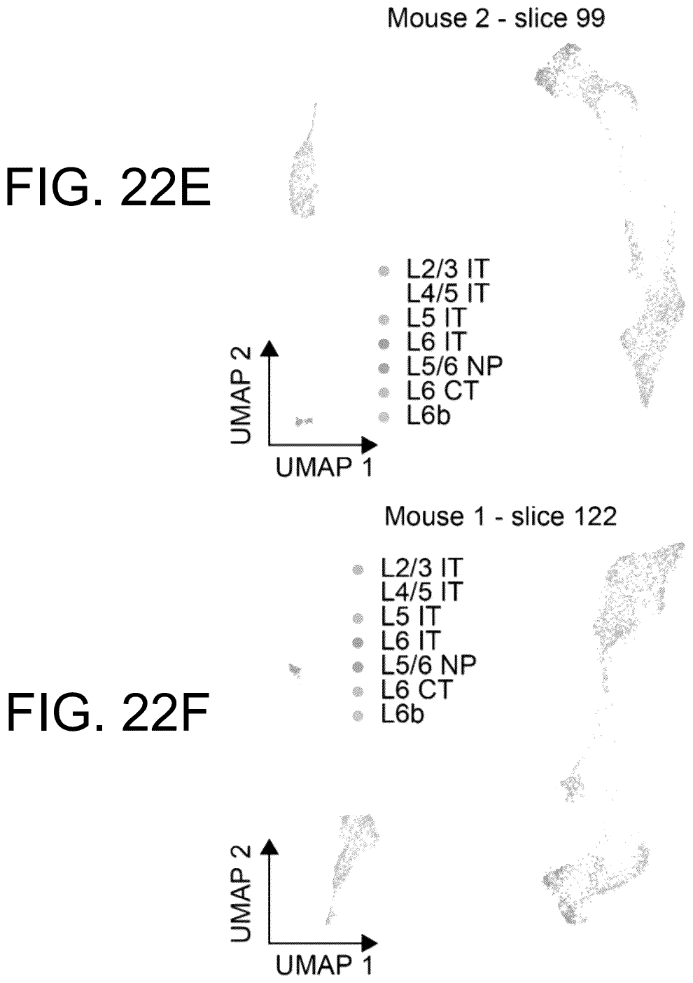

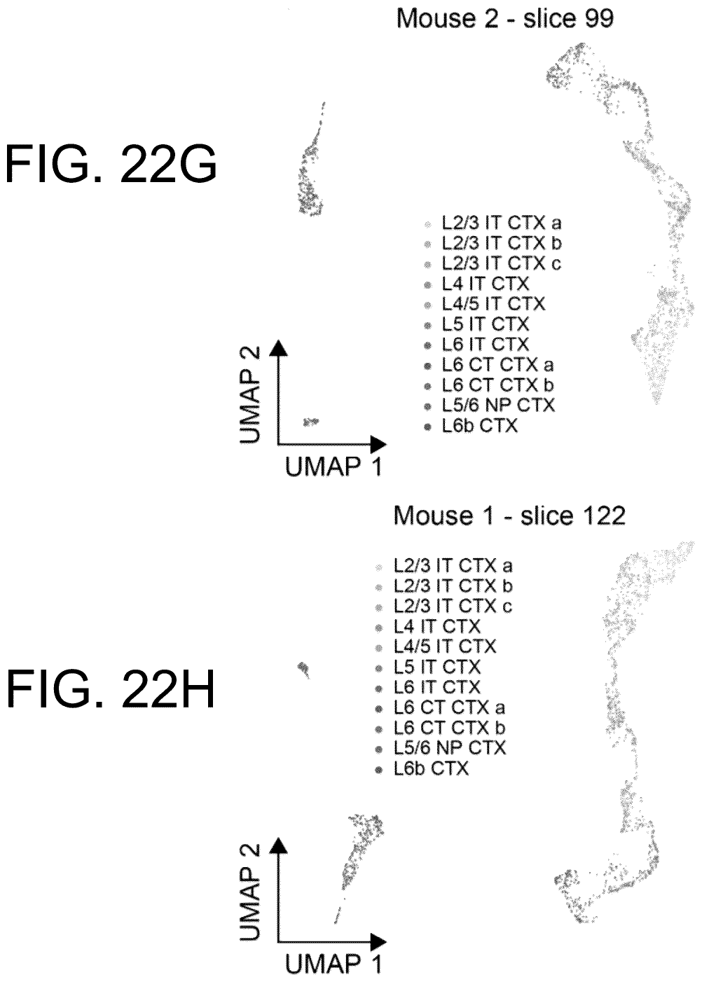

FIGS. 22A-22H illustrate membership correspondence between GAGE-seq and MERFISH datasets. Two tissue slices with the MERFISH dataset (Cardozo Gizzi A M, et al. Mol Cell 2019; 74:212-222) are shown. (22A, 22C, 22E, 22G). Slice 99 from mouse 2. (22B, 22D, 22F, 22H) Slice 122 from mouse 1. (22A, 22B) Adjacency matrix of the nearest neighbor graph of the integrated embedding space, aggregated by cell type. For each cell from the MERFISH dataset, its 20 nearest neighbors from the GAGE-seq dataset were included. (22C-22H) UMAP visualization of the integrated embedding space. (22C, 22D) Cells from both datasets, colored by dataset. (22E, 22F) Cells from the MERFISH dataset, colored by the cell type annotation from the original analysis. (22G, 22H) Cells from the GAGE-seq dataset, colored by the cell type annotation.

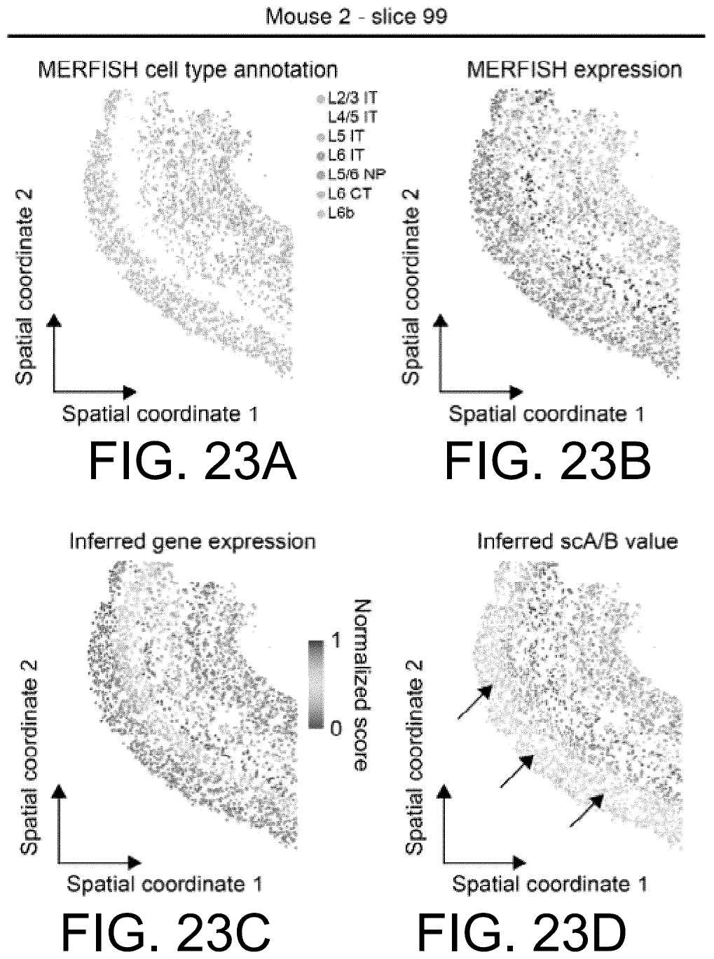

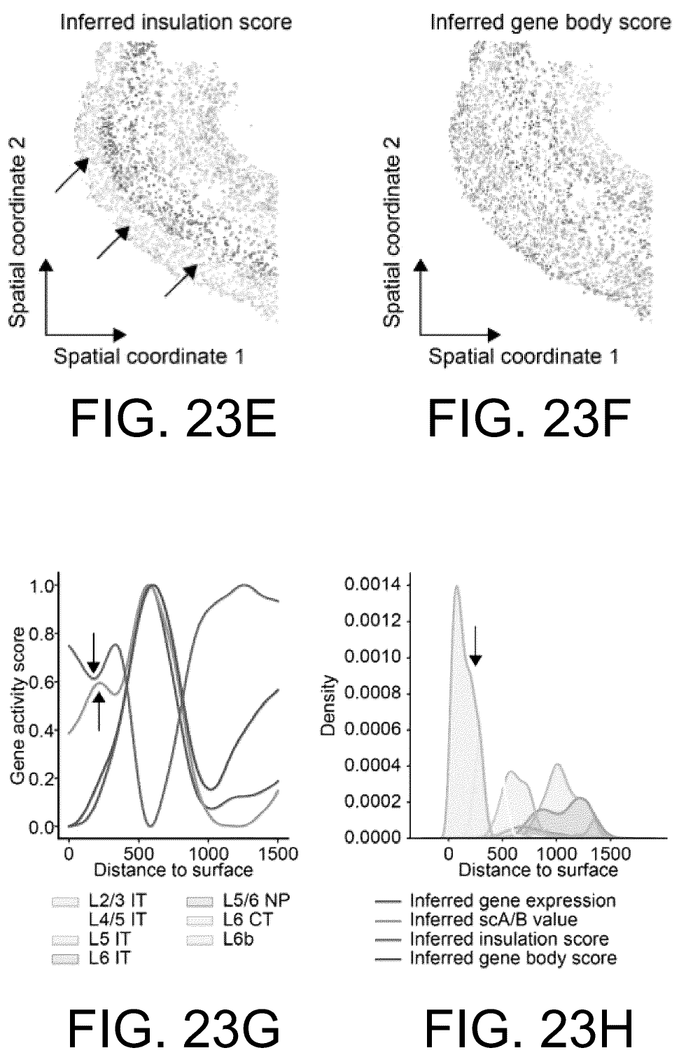

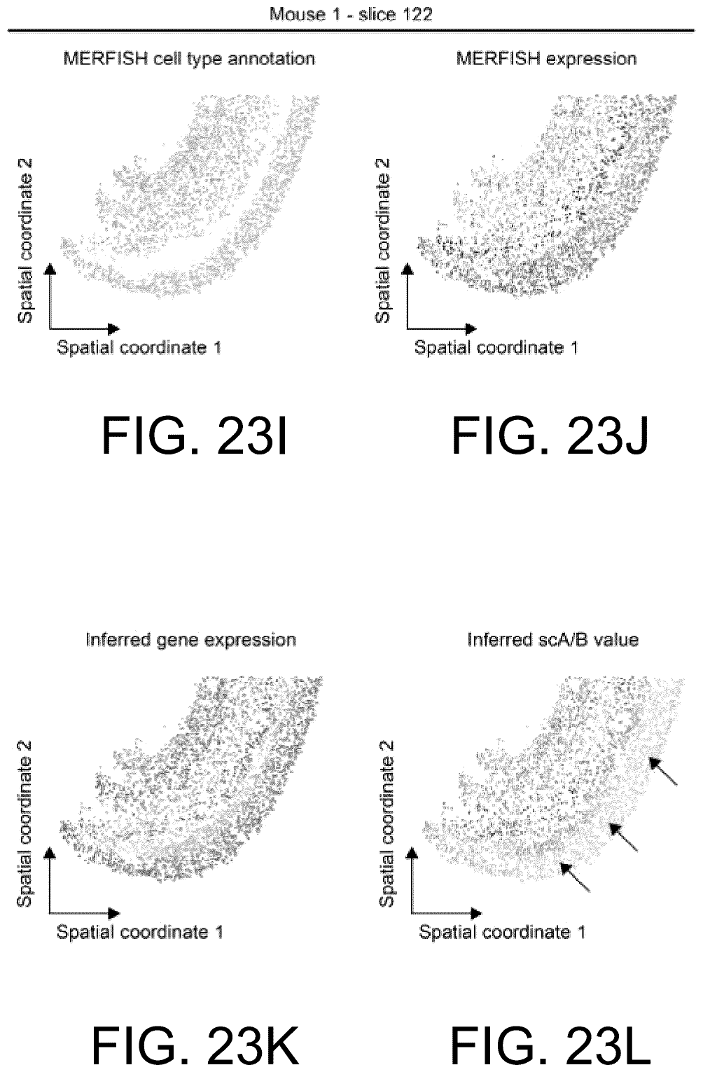

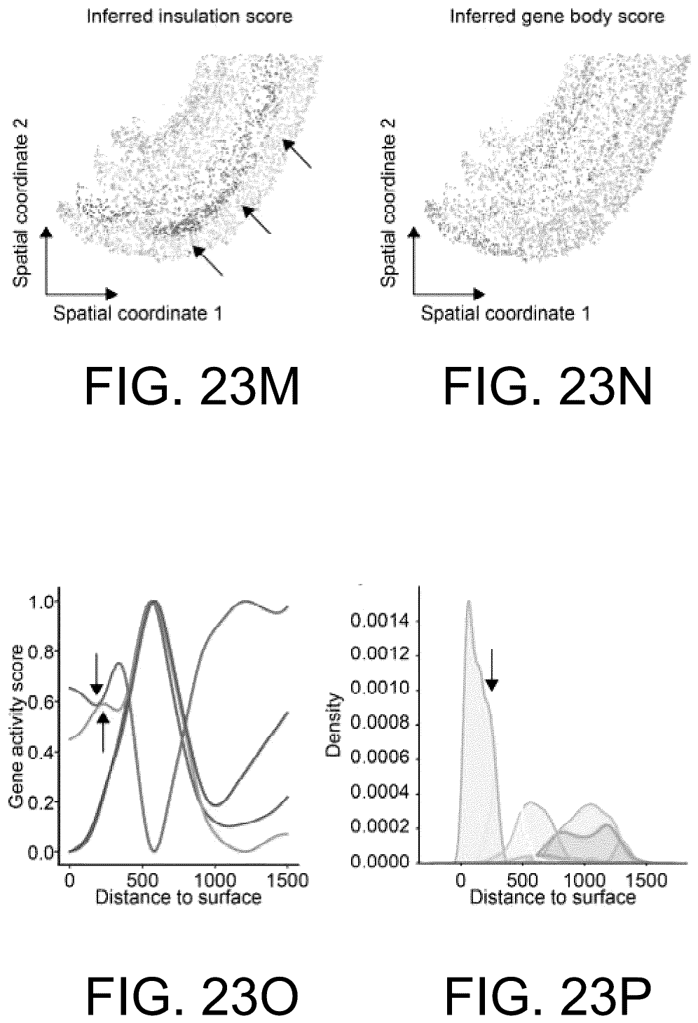

FIGS. 23A-23P illustrate high correlation between cortical layer-specific gene expression and the in situ dynamics of the 3D genome features of excitatory neurons. In situ plots of two tissue slices with the MERFISH dataset (Cardozo Gizzi A M, supra) are shown. (23A-23H) Slice 99 from mouse 2. (23i-23p) Slice 122 from mouse 1. (23A, 23I) in situ plot of cell type annotations from the original analysis. In panels (23B-23G) and (23J-230), multiple activity scores of L5 IT CTX marker genes are shown. Activity scores were averaged across genes for each cell. (23B, 23J) Detected expression level. (23C, 23K) Inferred expression level. (23D, 23L). Inferred scA/B value. (23E, 23M) Inferred single-cell insulation score. (23F, 23N) Inferred gene body score. (23G, 23O) The spatial gradient of different features with respect to the distance to the surface. (23H, 23P) The distribution of cell types with respect to the distance to surface.

The excitatory neuron cell types present in both GAGE-seq and MERFISH datasets were focused on. Within the integrated embedding space, cells primarily clustered by cell type, and cells from both datasets integrated cohesively, indicating high correlation between cell types identified by the two methods (FIGS. 16E, 22A-22H). Next the in situ variation of both marker gene expression and 3D genome features of these maker gene loci in the mouse cortex were characterized. As a proof of principle, the in situ pattern of marker genes was investigated for L5 intratelencephalic (IT) CTX. The observed and inferred gene expression demonstrated a high degree of congruence, further supporting the reliability of the integration (Spearman's r=0.76, two-sided P=0; FIGS. 23B, 23C, 23J, 23K). Layer 5, where L5 IT CTX cells reside, corresponded with the highest expression level, scA/B value27, gene body score (Supplementary Methods), and a low single-cell insulation score (FIGS. 16F, 16G, 23A-23P), reinforcing the overall correlation between expression and 3D genome structure. Despite consistently low expression levels and gene body scores in more superficial layers, the scA/B value increased and the single-cell insulation score decreased slightly around layer 3, a cortical layer containing the L2/3 IT CTX cells that are not adjacent to the tissue boundary, suggesting potential discrepancies of expression and various 3D genome features at finer spatial resolution (highlighted by arrows in FIGS. 16F, 16G, 23A-23P).

### Impact of 3D Genome on Gene Expressions in Single Cells.

Next the relationship between gene expression and various multiscale 3D genome features was rigorously examined in single cells, including A/B compartments, TAD-like domains, and chromatin loops.

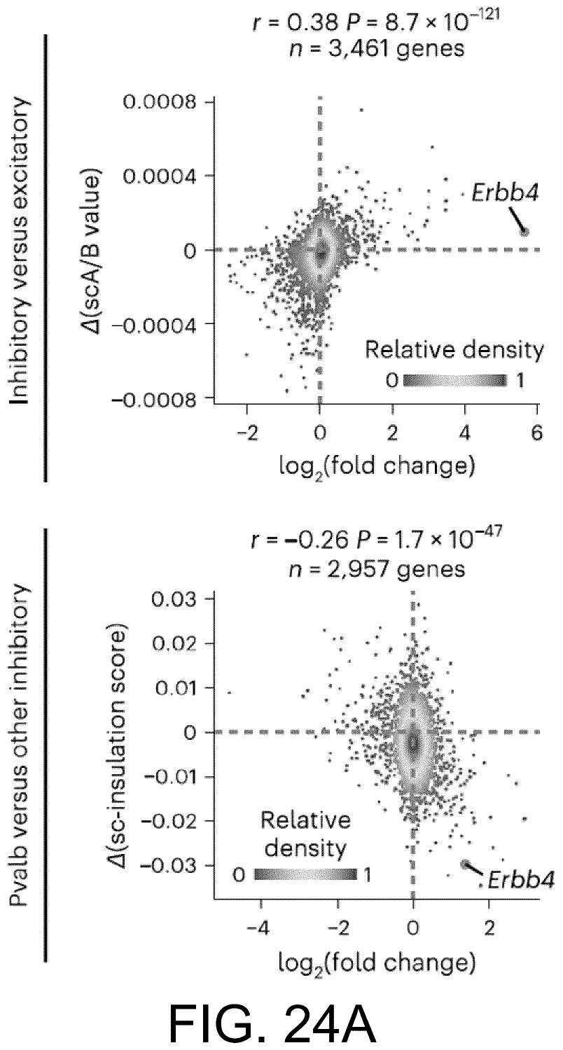

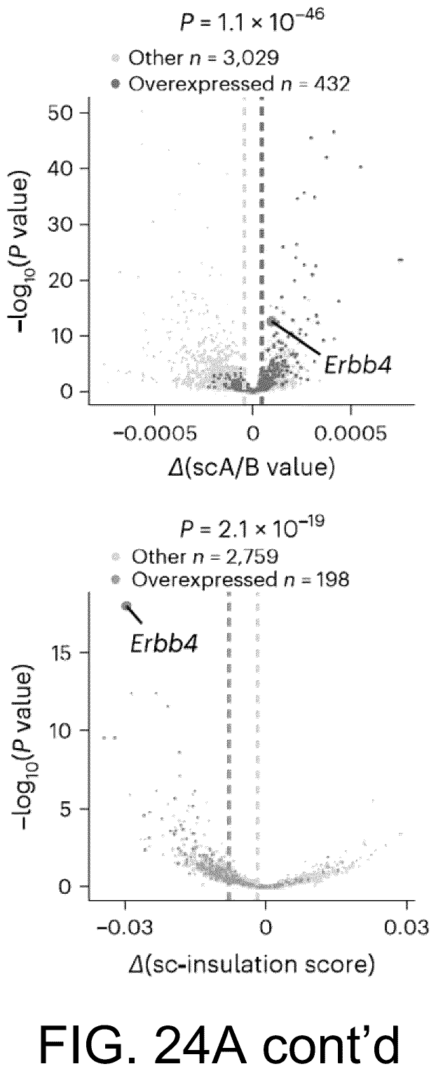

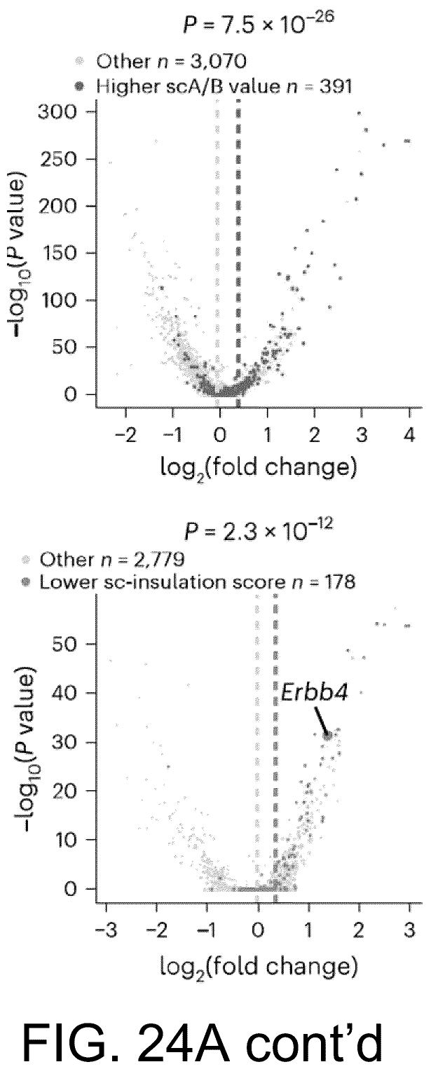

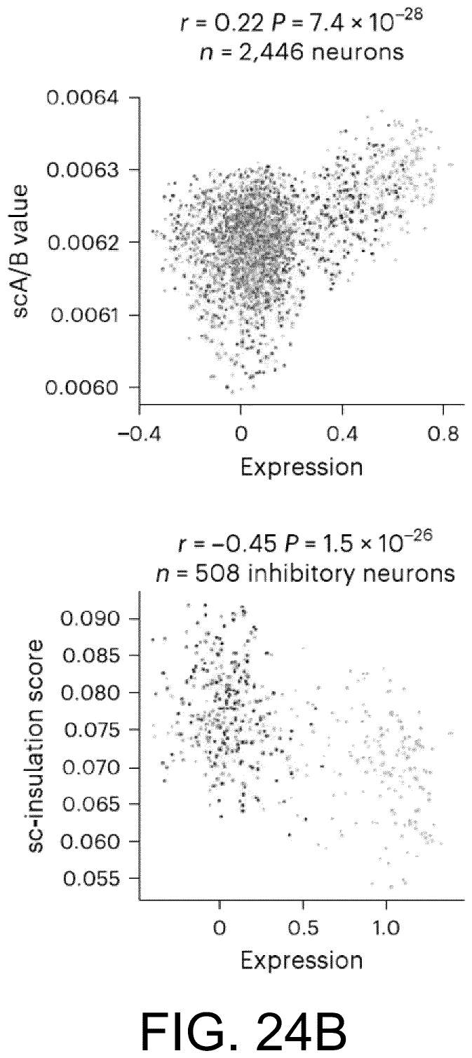

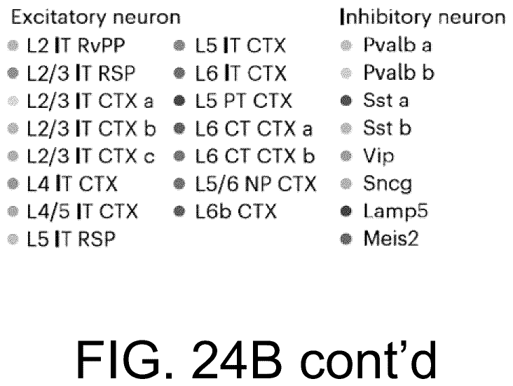

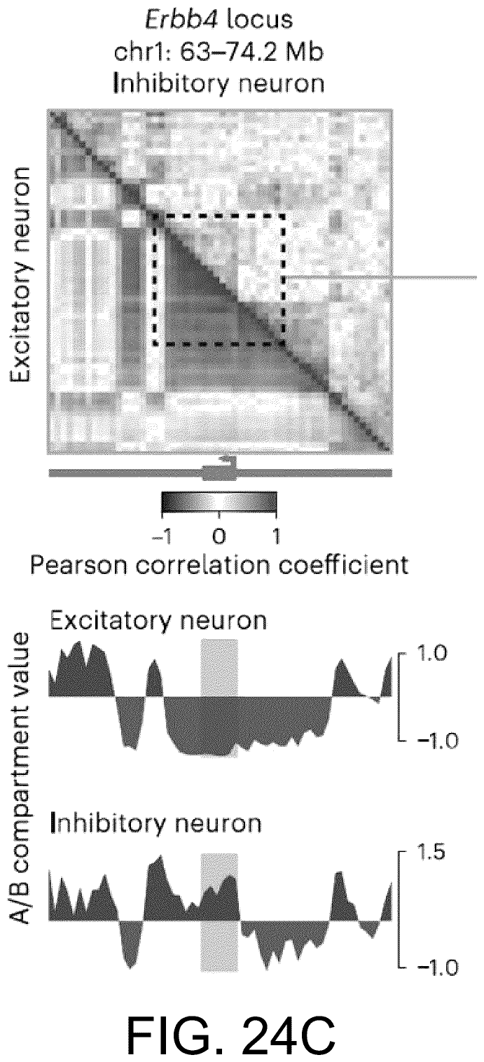

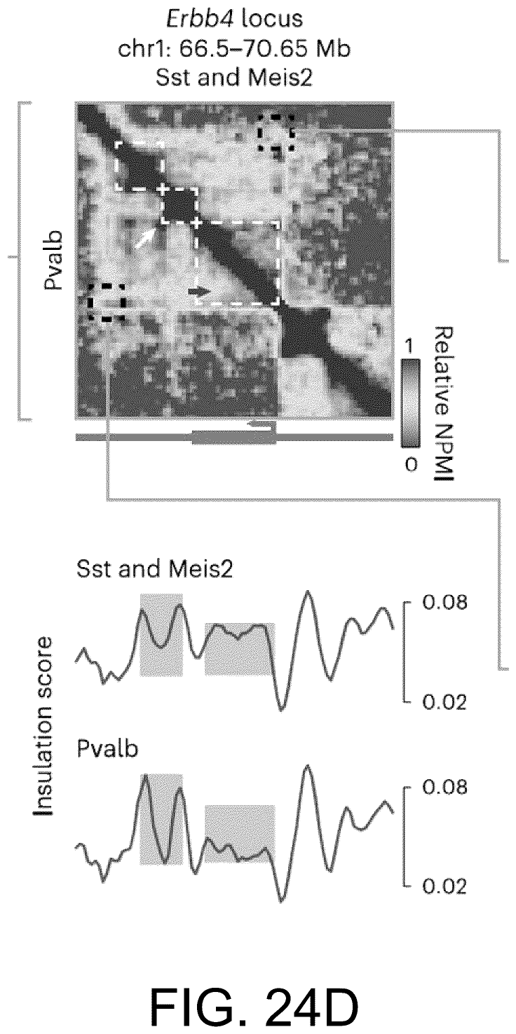

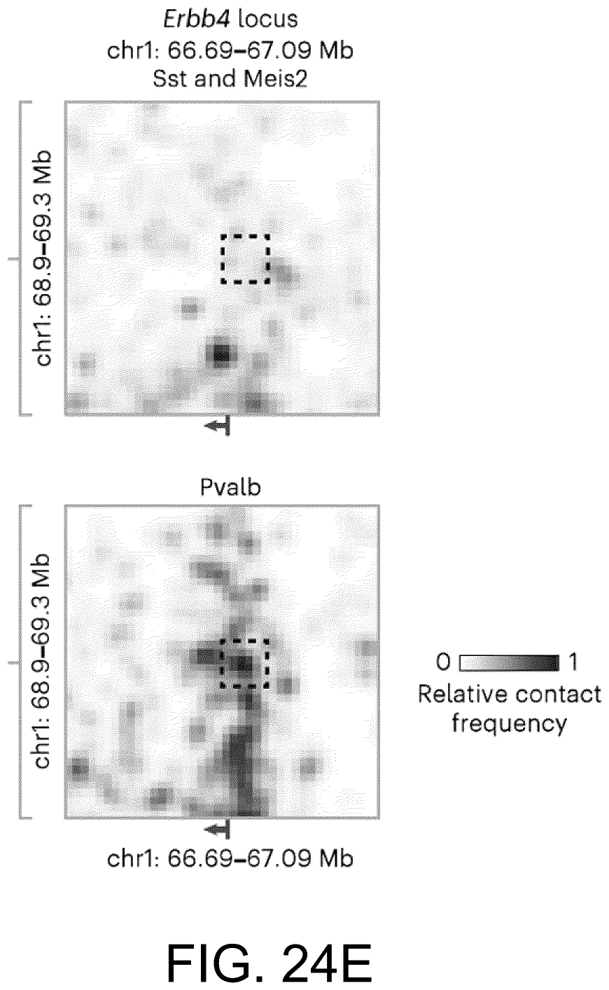

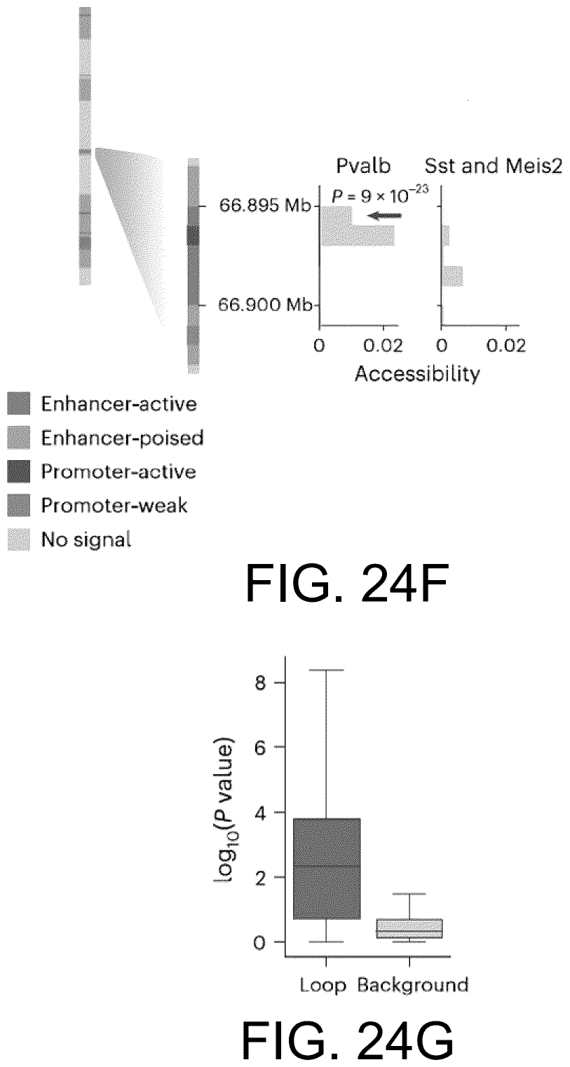

FIGS. 24A-24G illustrate that 3D genome features inform cell type-specific gene expressions in the mouse cortex. (24A) Correlations between gene expression and 3D genome features across neuron cell types. Upper row: inhibitory (n=508) vs. excitatory (n=1938). Lower row: Pvalb (n=188) vs. other inhibitory (n=320). First column: correlation between differential expression and differential 3D genome feature (Pearson's correlation coefficients and the P-values from one-sided tests for nonzero correlations shown). Second column: volcano plot of differential scA/B value and single-cell insulation score; Third column: volcano plot of differential expression. P-values from one-sided t-tests with unequal variance are shown in middle and right columns. (24B) Single-cell level correlation of gene expression with scA/B value (upper) or insulation score (lower) in inhibitory neurons (432 genes) and Pvalb (198 genes), respectively (Spearman's correlation coefficients and the P-values from one-sided tests for nonzero correlations shown). (24C) Comparison of A/B compartment (200-Kb resolution) of the Erbb4 locus between inhibitory and excitatory neurons. Pearson's correlation matrices of aggregated contact maps (top) and the A/B compartment scoretracks (bottom) are shown. (24D) Comparison of the pseudo-bulk contact map (50-Kb resolution) of the Erbb4 locus between Pvalb and other inhibitory subtypes. Pseudo-bulk contact maps (upper) and the insulation scores (bottom) are displayed. Two Pvalb-specific strides (white arrow) and melted TAD (black arrow) are shown in the top panel. The gene body is shown right under the contact matrices in (24C) and (24D), while the bottom panels highlight differential 3D genome features with light red boxes. (24E) Loop example in Pvalb (lower) and Sst and Meis2 (upper) inhibitory subtypes at 10-Kb resolution. Aggregated contact maps, regulatory element annotations52 (right), and TSS of Erbb4 (bittin arrow) are shown. (24f) Differential accessibility around the enhancer in Pvalb (left) vs. Sst and Meis2 (right), with a 1 kb enhancer region highlighted (black arrow). The P-values of one-sided Mann-Whitney U tests are shown. (24G) Loop vs. non-loop contacts correlation with expression. P-values from two-sided tests for nonzero Spearman's correlation coefficients are shown (n=3,105 cells).

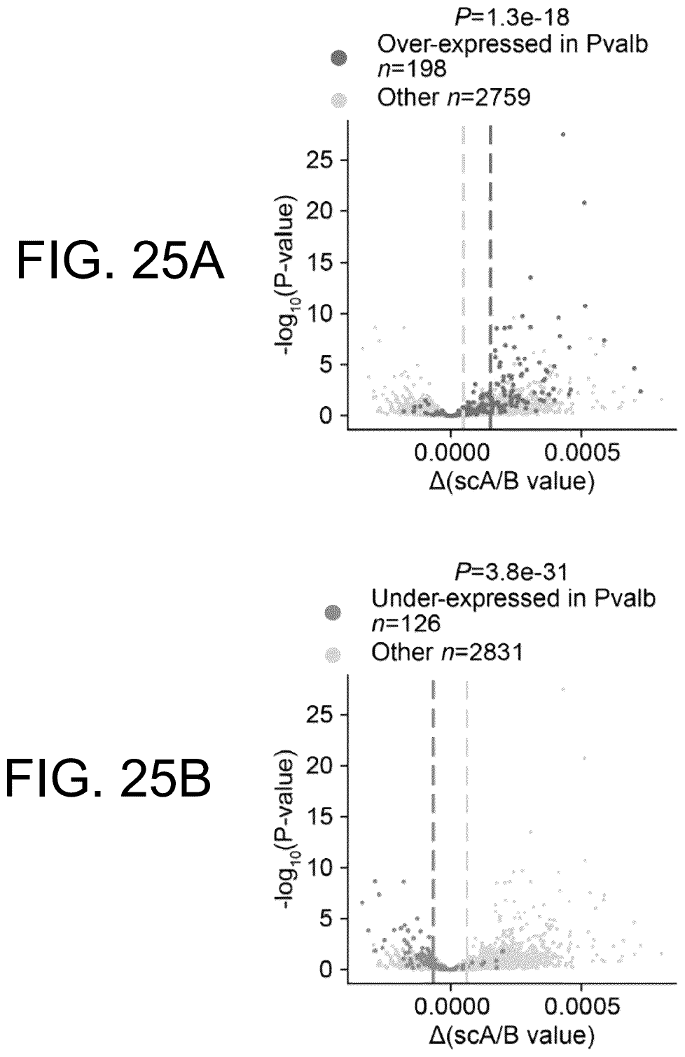

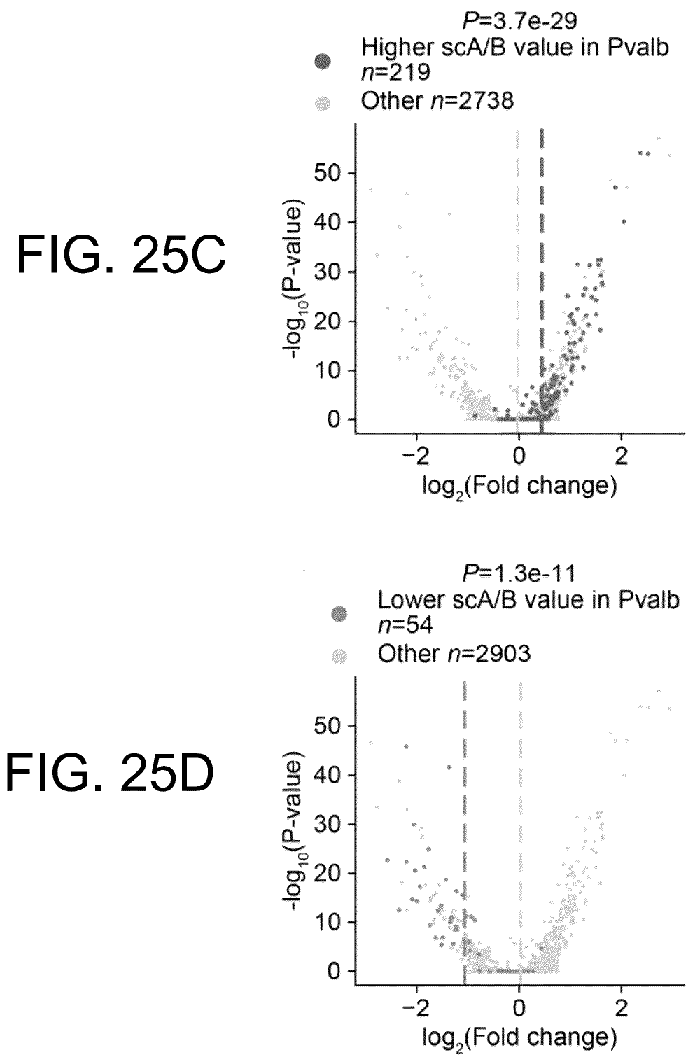

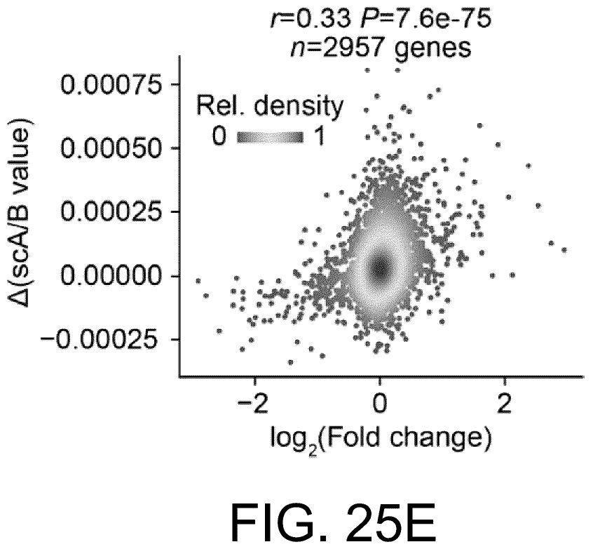

FIGS. 25A-24E illustrate correlation between cell type-specific single-cell A/B value and gene expression when comparing Pvalb and the other inhibitory neurons. (25A, 25B) Volcano plot of differential scA/B value. (25C, 25D) Volcano plot of differential gene expression. (25E) The whole-transcriptome correlation between differential expression and differential scA/B value. The CpG-based scA/B value was used. P-value of one-sided t-test is shown in 25A-25D. Pearson's correlation coefficient and one-sided P-value are shown in 25E.

The analysis of the 3,461 genes expressed in inhibitory neurons (n=508) or excitatory neurons (n=1,938) revealed a strong correlation between cell type-specific gene expression and scA/B value, reflecting compartmentalization variations (Tan L, et al. Cell, supra; Zhang R, supra) (FIG. 24A, top panels). Inhibitory neurons, for instance, showed a much higher expression for 432 genes which corresponded to a higher scA/B value (t-test P=1.1e-46; FIGS. 24A, top middle panel). Most of the 391 genes with a higher scA/B value in inhibitory neurons also snowed notably higher expression levels in these cells compared to excitatory neurons (t-test P=7.5e-26, FIG. 24A, top right panel). Overall, there is a significant correlation between differential gene expression and differential scA/B value (Pearson's r=0.38, P<1e-100, FIG. 24A, top left, FIGS. 25A-25E). At the chromatin domain level, a negative correlation between cell type-specific gene expression and the associated single-cell insulation score was identified across cell types (FIG. 24A, bottom panels), suggesting that TAD-like domain variations around the gene body are accompanied with changes in transcriptional activity of the gene. This phenomenon, aligning with previous findings at the cell type level (Zhang R, supra), may be attributed to domain melting noted in highly expressed long genes in mouse hippocampus and midbrain neurons (Winick-Ng W, supra).

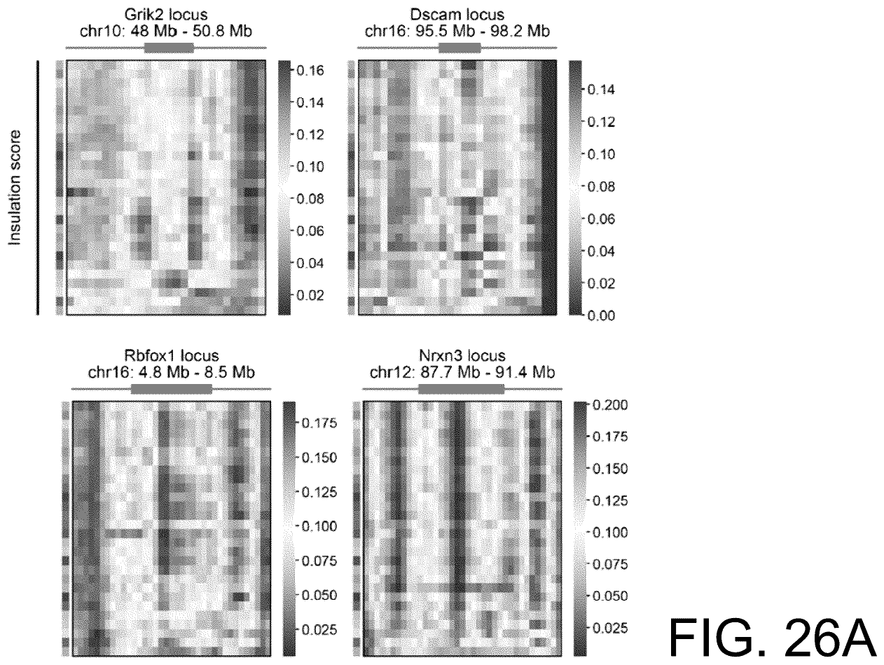

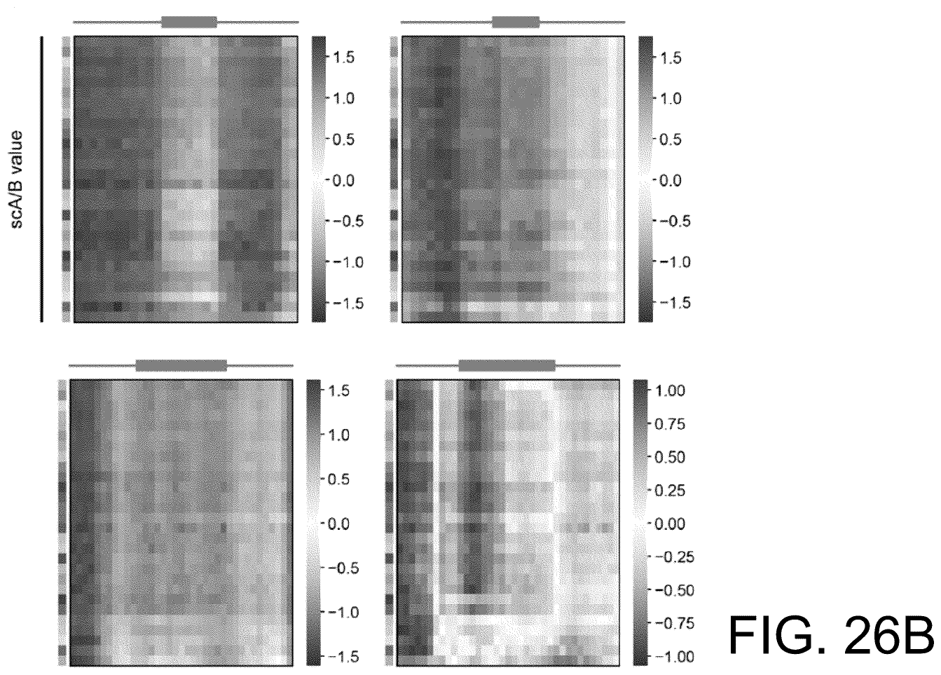

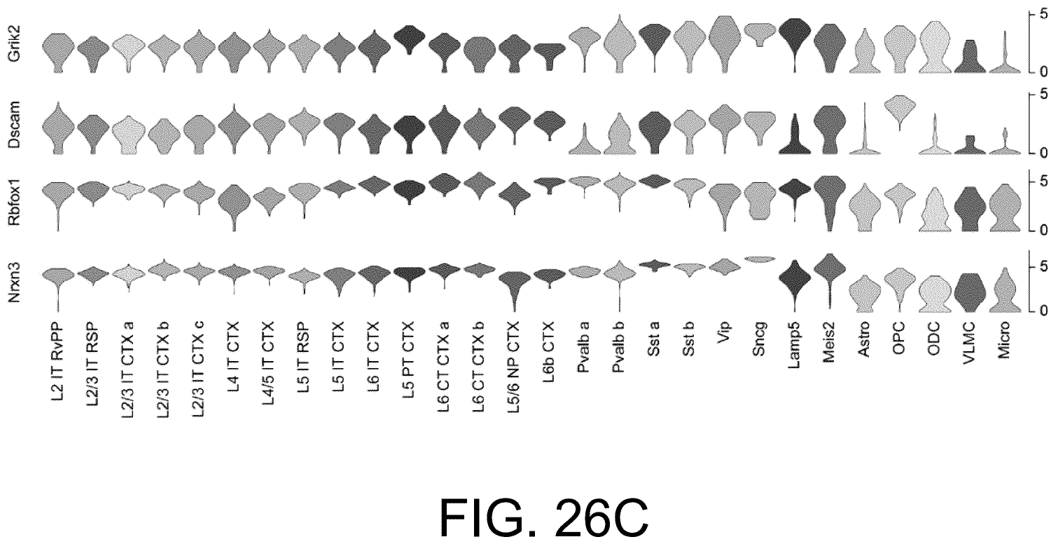

FIGS. 26A-26C illustrate aggregated single-cell insulation score and scA/B value of the four gene loci, Grik2, Dscam, Rbfox1 and Nrxn3 in the annotated 28 cell subtypes. (26A) Aggregated single-cell insulation score calculated on raw contact maps. (26B) Z-scored eigenvector-based scA/B value calculated on Higashi-imputed contact maps. (26C) Violin plots showing single-cell gene expressionprofiles of the four genes. Gene bodies are shown as gray boxes above heat maps.

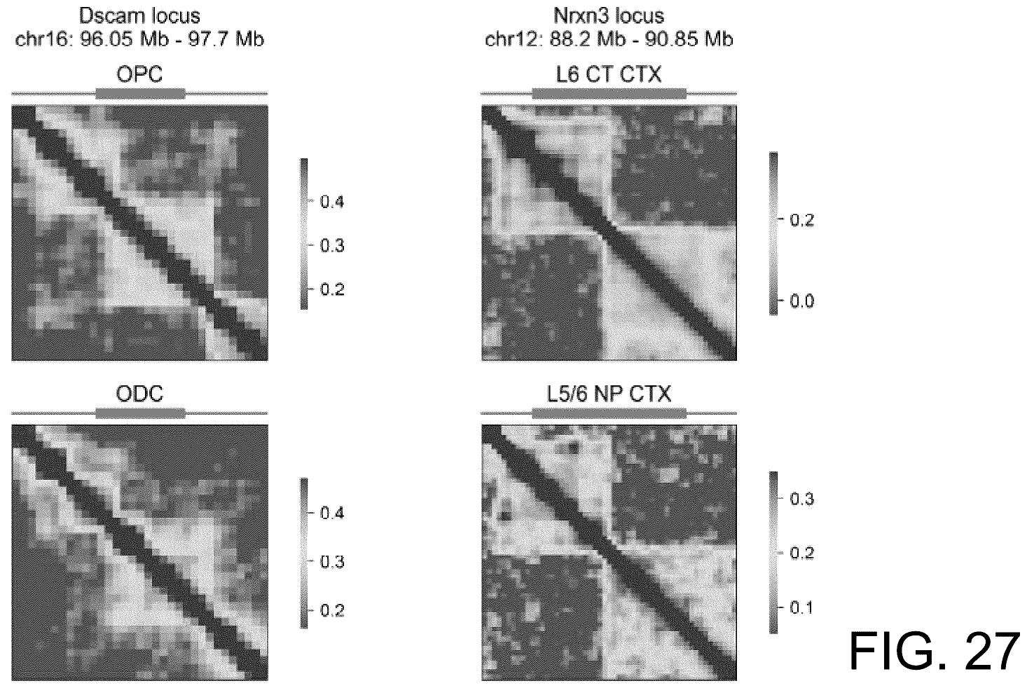

FIG. 27 illustrates aggregated contact maps of the Dscam and Nrxn3 gene loci showing cell type-specific domain organization. For each locus, two cell types with differential expression were selected and the two aggregated contact maps are shown. All contact maps are at the 50 Kb resolution and are normalized by NPMI. Gene bodies are shown as gray boxes above heat maps.

Subsequently the relationship between single-cell insulation score surrounding the gene body and the potential occurrence of domain melting was investigated within the diverse collection of cell types revealed by GAGE-seq. The four genes (Grik2, Dscam, Rbfox1, and Nrxn) known to undergo domain melting were focused on (Winick-Ng W, supra), profiling their scA/B value, single-cell insulation score, and single-cell gene expression. Notably, these genes manifested high expression across almost all 28 cell subtypes revealed by GAGE-seq, with the exception of Dscam and Grik2 in VLMC and Micro cells (FIGS. 26A-26C, 16D). Dscam, Rbfox1, and Nrxn3 were predominantly in the active A compartment in the majority of cell subtypes (FIGS. 26A-26C, 16D), while the Grik2 locus was in a weak B compartment across all the cells, despite its high expression (FIGS. 26A-26C). Aggregated single-cell insulation scores varied across the gene body, with most cell subtypes showing lower scores correlating with elevated gene expression (FIGS. 26A-26C, 16D). The aggregated chromatin contact maps indicate potential occurrence of domain melting around these gene bodies (FIGS. 16D, 27). A similar phenomenon was also detected for the Rbfox1 locus across different excitatory neurons (FIG. 16D, low panels).

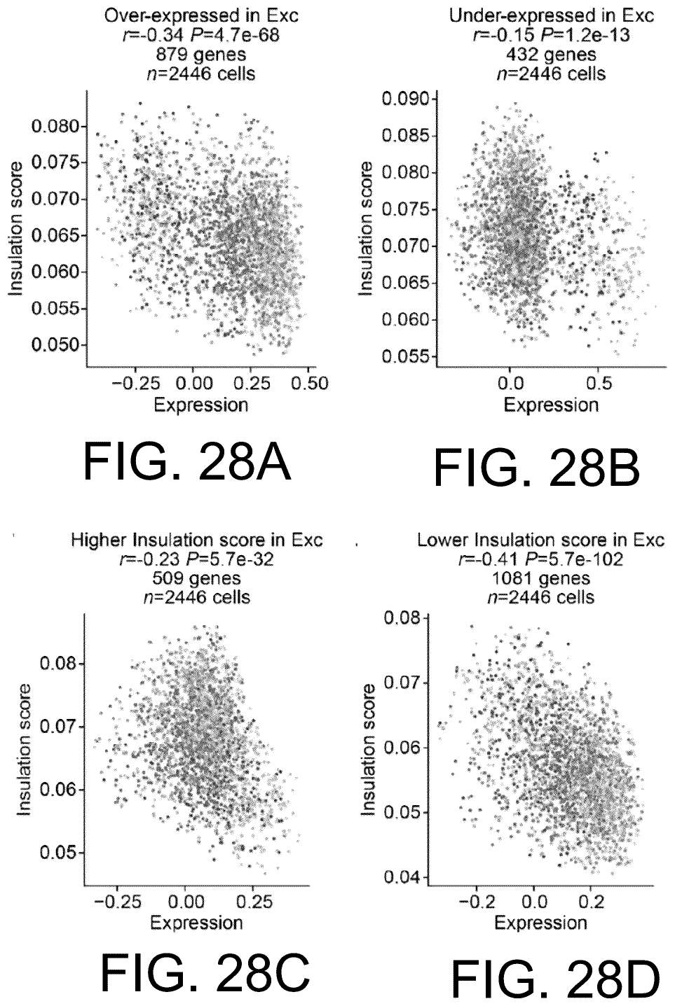

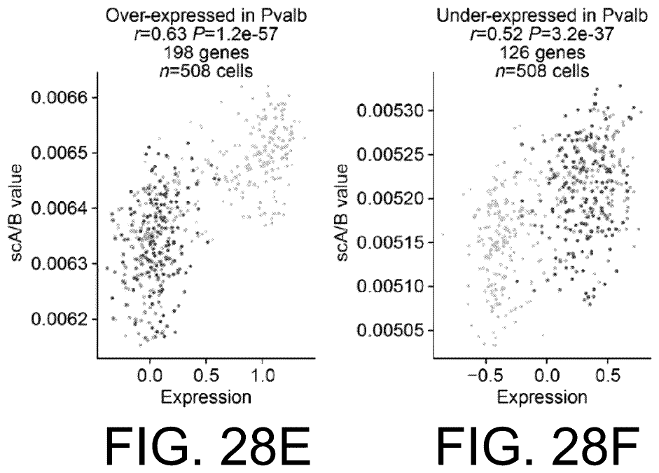

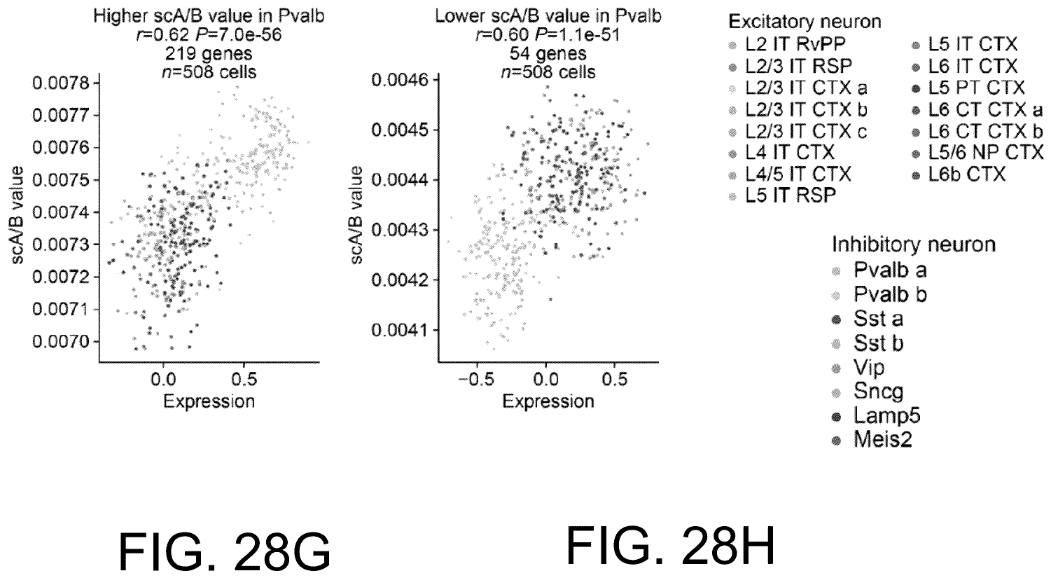

FIGS. 28A-28H illustrate the correlation between gene expression and 3D genome features at the single-cell level. For each cell, the average expression and 3D genome features of a particular set of genes are shown. (28A-28D) The correlation between expression and single-cell insulation score. (28E-28H) The correlation between expression and CpG-based scA/B value. Each cell type was denoted by a distinct color shown at the bottom. The gene set in each panel: (28A) Genes over-expressed in excitatory neurons; (28B) Genes under-expressed in excitatory neurons; (28C) Genes having higher sc-insulation scores in excitatory neurons; (28D) Genes having lower sc-insulation scores in excitatory neurons; (28E) Genes overexpressed in Pvalb; (28F) Genes under-expressed in Pvalb; (28G) Genes having higher scA/B values in Pvalb; (28H) Genes having lower scA/B in Pvalb. Pearson's correlation coefficients and the P-values for one-sided test are shown.

Next the above observed connection between multiscale 3D genome features and gene expression was further confirmed at single cell resolution. Higher gene expression in a cell often corresponded to a higher scA/B value and lower single-cell insulation score in the same cell (FIG. 24B, 28A-28H). For instance, of the 432 genes showing a significantly elevated scA/B value in inhibitory neurons, most displayed higher expression in these neurons than in excitatory neurons (Spearman's r=0.22, P=7.4e-28, n=2446 cells; FIG. 24B, top panel). At the chromatin domain level, the 198 genes expressed highly in Pvalb cells exhibited notably lower single-cell insulation scores than in other inhibitory neurons (Spearman's r=0.45, P=1.5e-26, n=508 cells; FIG. 24B, low panel). Thus, the connection between multiscale 3D genome features and gene expression is evident at the single-cell resolution.

The observations were then confirmed on single loci. As a proof of principle, the Pvalb inhibitory subtype was focused on (including both Pvalb a and Pvalb b). First genes were selected that have 1) significantly higher scA/B values and expression in inhibitory neurons compared to excitatory neurons (FIG. 24A, top panels), and 2) significantly higher expression and lower single-cell insulation scores in Pvalb compared to other inhibitory neurons (FIG. 24A, bottom panels). This approach led us to the Erbb4 gene. The Erbb4 gene plays a pivotal role in the central nervous system and has been linked to schizophrenia (Law A J, et al. Hum Mol Genet 2007; 16:129-41). Differential A/B compartment states correlated with cell type-specific expression of the Erbb4 gene were observed (FIG. 24C), and differential single-cell insulation score that suggests domain melting in the gene locus (FIG. 24D, low panel). The TAD-like domain structure of the Erbb4 gene body in Sst and Meis2 cells appears to be melted in Pvalb cells (i.e., less pronounced), which is again accompanied with high gene expression in Pvalb cells (FIG. 24D, top panel). Additionally, it appears that the Erbb4 gene body interacts more frequently with the downstream two small TAD-like domains in Pvalb cells than in Sst and Meis2 cells (FIG. 24D, top panel). On a finer scale, a cell type-specific putative enhancer-promoter chromatin loop at the TSS of the Erbb4 gene in Pvalb cells was also observed (FIGS. 24E-24G). Moreover, when integrating with chromatin accessibility, the putative enhancer region exhibits differential chromatin accessibility that correlates with the cell type-specific expression of the Erbb4 gene (FIG. 24F).

### Integrative Analysis of GAGE-Seq and Chromatin Accessibility.

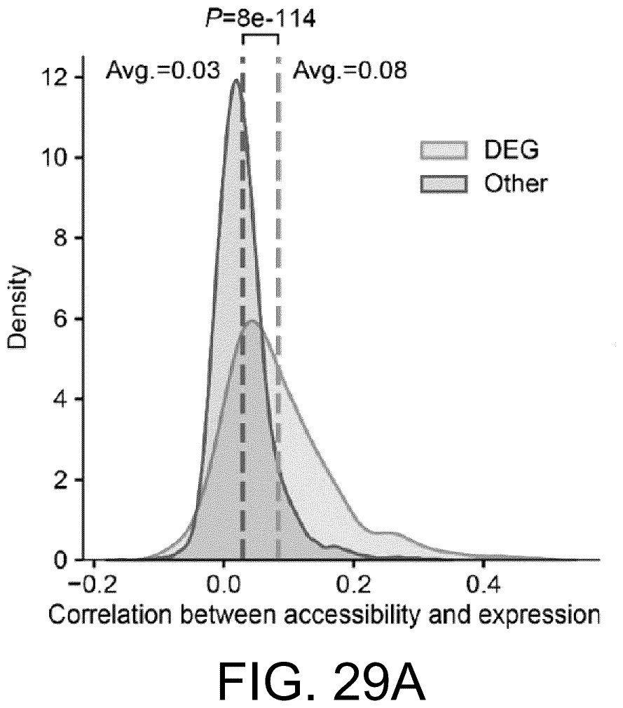

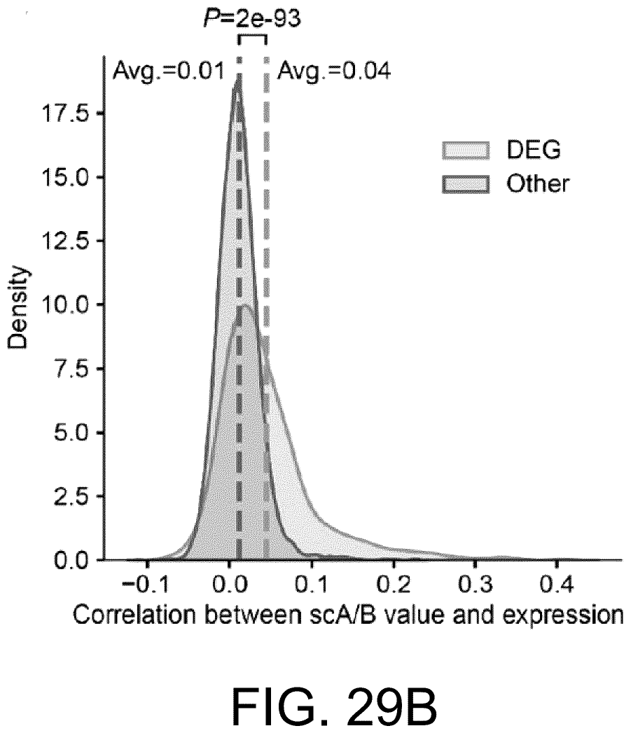

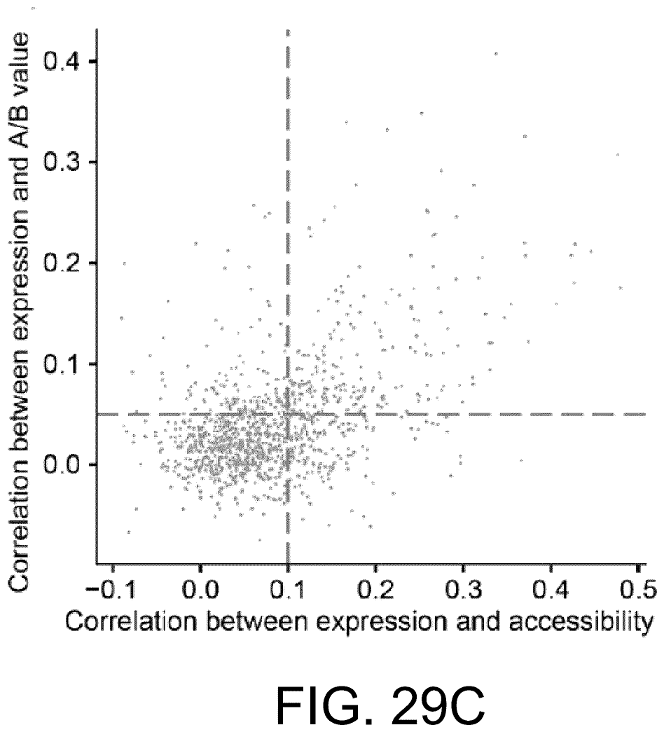

FIGS. 29A-29C illustrate the joint influence of A/B compartment and chromatin accessibility on gene expression. (29A) Correlation coefficients between accessibility and expression of highly variable genes (n=6,472). Differentially expressed genes (DEGs) between Exc and Inh (n=1,066; orange) have significantly stronger correlation than non-DEGs (n=5,405; blue). (29B) Correlation coefficients between scA/B value and expression of highly variable genes (n=6,472). DEGs between Exc and Inh (n=1,066; orange) have significantly stronger correlation than non-DEGs (n=5,405; blue). In 29A, 29B, the Pvalues of two-sided Mann-Whitney U tests are shown at the top of panels (29A) and (29B). (29C) Partitions of genes jointly based on accessibility-expression correlation and scA/B-expression correlation. The cutoff of accessibility-expression correlation is set to 0.1, and the cut-off of scA/B-expression correlation is set to 0.05. The expressions of n=218 genes are highly correlated with both scA/B and accessibility (upper right partition), the expressions of n=142 genes are highly correlated with scA/B but are not correlated with accessibility (upper left partition), the expressions of n=142 genes are highly correlated with accessibility but are not correlated with scA/B (lower right partition), and the expressions of n=564 genes are not correlated with scA/B nor with accessibility (lower left partition).

It was next aimed to demonstrate how integrating GAGE-seq with chromatin accessibility data enhances the connection between CREs and target genes. For this, GAGE-seq was integrated with Paired-seq data (from the same mouse cortex region) (Zhu C, supra). Overall, genes with distinct contributions from 3D genome and chromatin accessibility show varied functions (FIGS. 29A-29C) and integrating 3D genome and chromatin accessibility data markedly improves gene expression prediction accuracy.

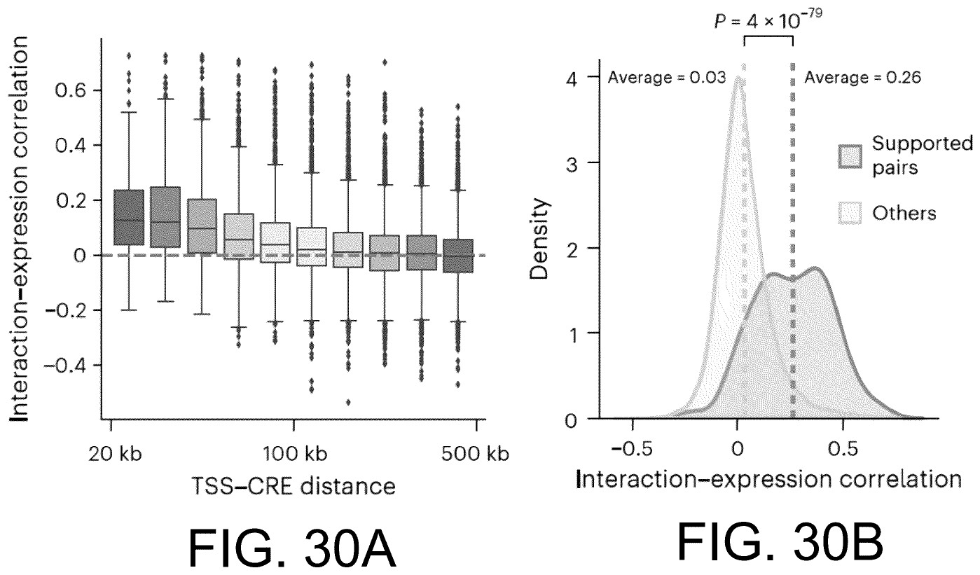

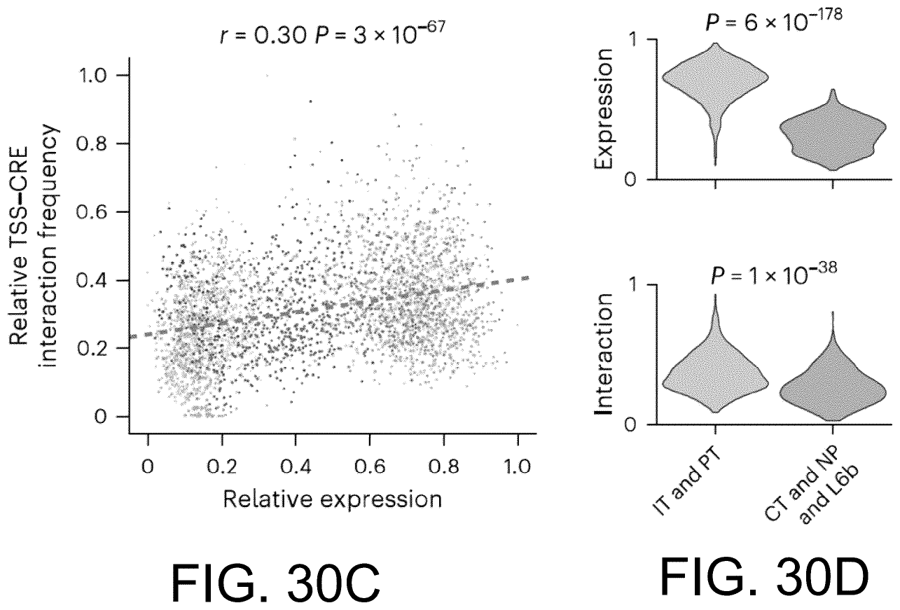

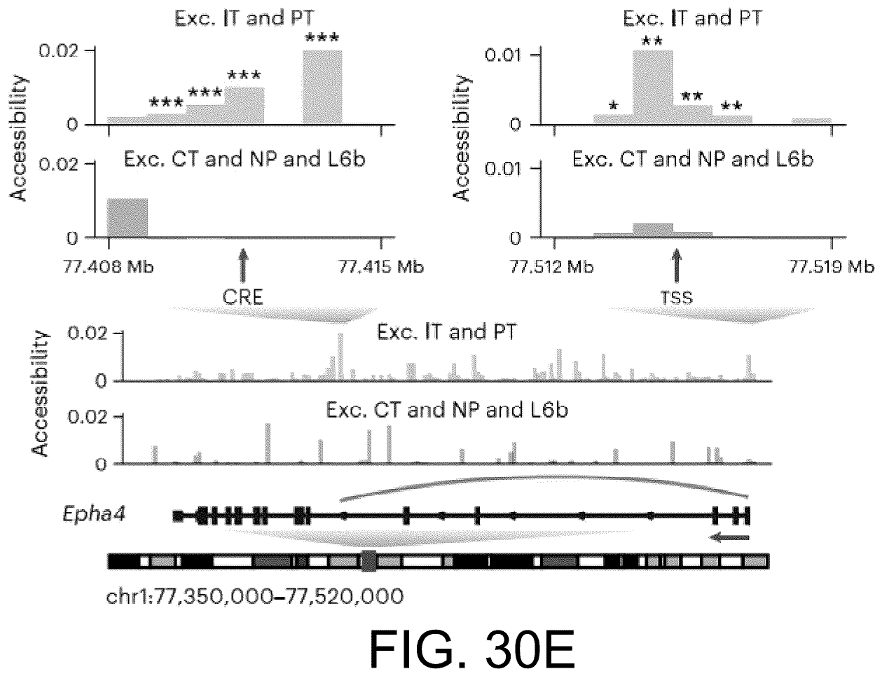

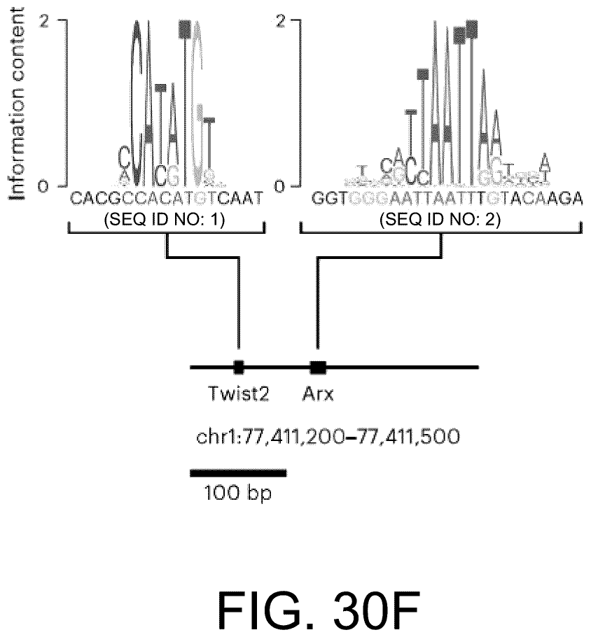

FIGS. 30A-30F illustrate integrative analysis of GAGE-seq and chromatin accessibility in the mouse cortex. (30A) Correlation coefficient (n=3,105 cells) between expression and TSS-CRE interaction frequency for each gene-CRE pairs from Paired-seq data63, grouped by genomic distance between TSS and CRE. (30B) Comparison between gene-CRE pairs corroborated by other sources (red) and those identified only from Paired-seq data63 (yellow). The P-value of two-sided Mann-Whitney U test is shown. (30C-30E) The combined effect of 3D genome and accessibility on expression at the Epha4 locus. (30C) Correlation of interaction-expression for a specific gene-CRE pair at the Epha4 gene, with dots representing single cells colored by cell type. (30D) Expression (upper) and TSS-CRE interaction frequency (lower) comparison among excitatory subtypes, revealing heightened levels in IT and PT subtypes. The P-values of one-sided Mann-Whitney U tests are shown. (30E) Accessibility comparison around the TSS and CRE (chr1: 77410959-77411960) of the Epha4 gene among excitatory subtypes, showing higher accessibility IT and PT subtypes. The P-values of two-sided Mann-Whitney U tests are shown. IT and PT subtypes are compared against CT, NP, and L6b subtypes in (30D) and (30E). In (30E), *: P<1e-3; **: P<1e-5; ***: P<1e-10; the P-values in the upper left plot are (from left to right): 2e-11, 7e-20, 8e-34, 7e-52; the P-values in the upper right plot are: 6e-4, 6e-8, 7e-6, 2e-6, 1e-4. (30F) Binding sites of transcription factors Twist2 and Arx at the CRE of the Epha4 gene, depicting both the canonical motif (top) and the identified binding motif sequence (bottom) for each TF.

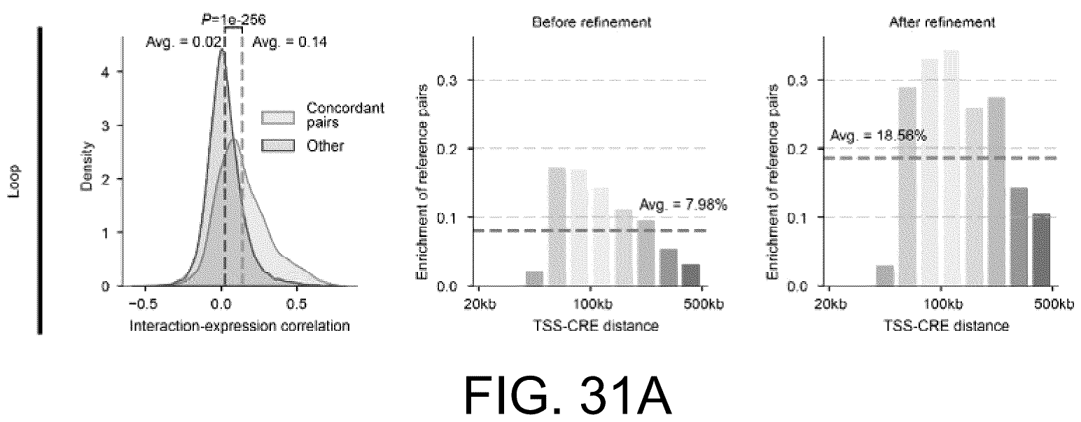

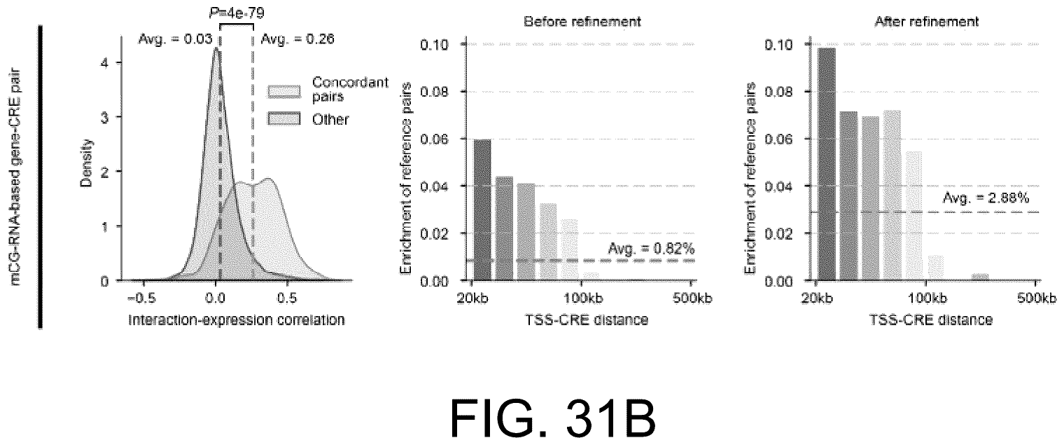

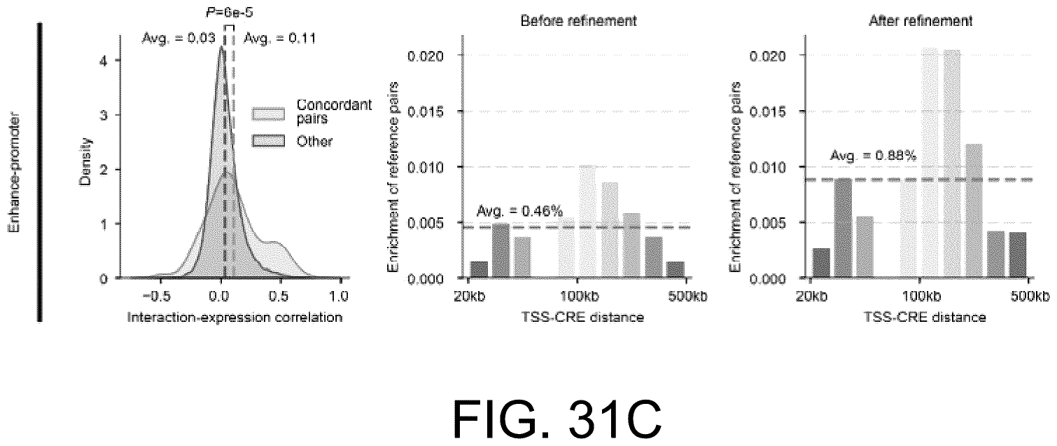

FIGS. 31A-31C illustrate the refinement of gene-CRE pairs enabled by GAGE-seq. Left column. The comparison between gene-CRE pairs supported by other approaches/sources (orange), i.e., orthogonal datasets and gene-CRE pairs identified only from the Paired-seq data (Misteli T. Cell 2020; 183:28-45) (blue). For gene-CRE pairs supported by other sources, expression and TSS-CRE interaction frequency generally have a stronger correlation. The P-values of two-sided Mann-Whitney U tests are shown. Middle column. The percentage of gene-CRE pairs identified from the Paired-seq data that are supported by other sources. Right column. The percentage of gene-CRE pairs identified from the Paired-seq data and refined by GAGE-seq that are supported by other sources. First row. The orthogonal dataset based on loop calling on the GAGE-seq data. Second row. The orthogonal dataset of gene-CRE pairs based on co-assayed sc-mCG and scRNA (Nagano T, supra). Third row. The orthogonal dataset of enhancer-promoter interaction annotated from mouse ENCODE histone modification datasets (Flyamer IM, supra).

The integrative analysis of GAGE-seq and chromatin accessibility enhances the connection of CREs to their target genes. The gene expression and transcription start site (TSS)-CRE interaction frequency correlation decreases with greater genomic distance between TSS and CRE (FIG. 30A). Also, overlaps between Paired-seq-identified gene-CRE pairs and those identified by other approaches generally decrease with increasing genomic distance between TSS and CRE (FIGS. 31A-31C). However, refining with GAGE-seq data markedly improved this overlap, particularly for long-range (>100 kb) gene-CRE pairs (FIGS. 30B, 31A-31C), highlighting the advantage of GAGE-seq in revealing CRE-gene pairs.

The joint regulation of gene expression by 3D genome and chromatin accessibility at individual gene loci was explored. A strong correlation was found between Epha4 gene expression and the chromatin interaction frequency with a distal CRE, as well as between Epha4 gene expression and chromatin accessibility at the TSS and the distal CRE in different excitatory neuron subtypes (FIGS. 30C-30E). Motif analysis of chromatin accessibility peaks identified potential binding sites for transcription factors Twist2 (Spearman's P=1e-289) and Arx (Spearman's P=2e-132) (FIG. 30F). However, no significant differences were noted for A/B compartment value, insulation score, and gene body score of the Epha4 locus across neuron subtypes, indicating that fine-scale CRE-chromatin looping instead of changes in the large-scale 3D features may be responsible for the cell type-specific Epha4 expression.

### Developmental Stages of Human Hematopoiesis.

Hematopoiesis is a classic model system with well-characterized cell type changes and their associated gene expression signatures, making it an ideal model for exploring the dynamic relationship between 3D genome structure and gene expression. GAGE-seq profiles of 2,815 human bone marrow (BM) CD34+ cells were generated after stringent quality filtering, obtaining an average of 265,336 chromatin contacts (at 50% duplication rate) and detecting on average 5,504 μMIs and 985 genes per cell (at 63% duplication rate), which is in line with the publicly available scRNA-seq datasets. To mitigate the potential impact of 3D genome's cell-cycle dynamics (Nagano T, supra), the analysis was restricted to high-quality GO/G1 phase cells (837 cells).

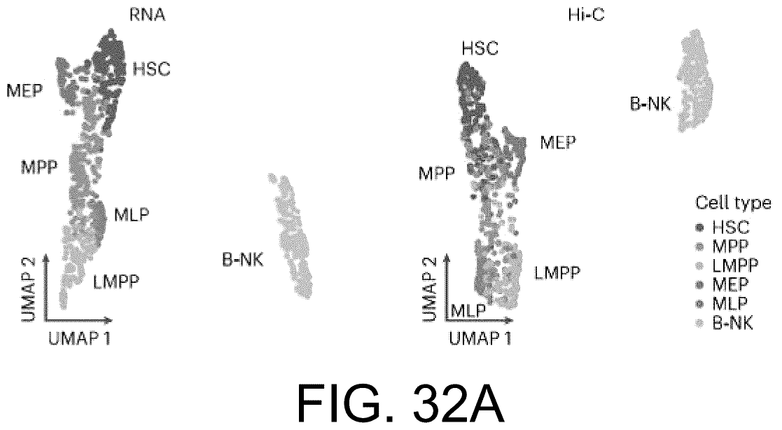

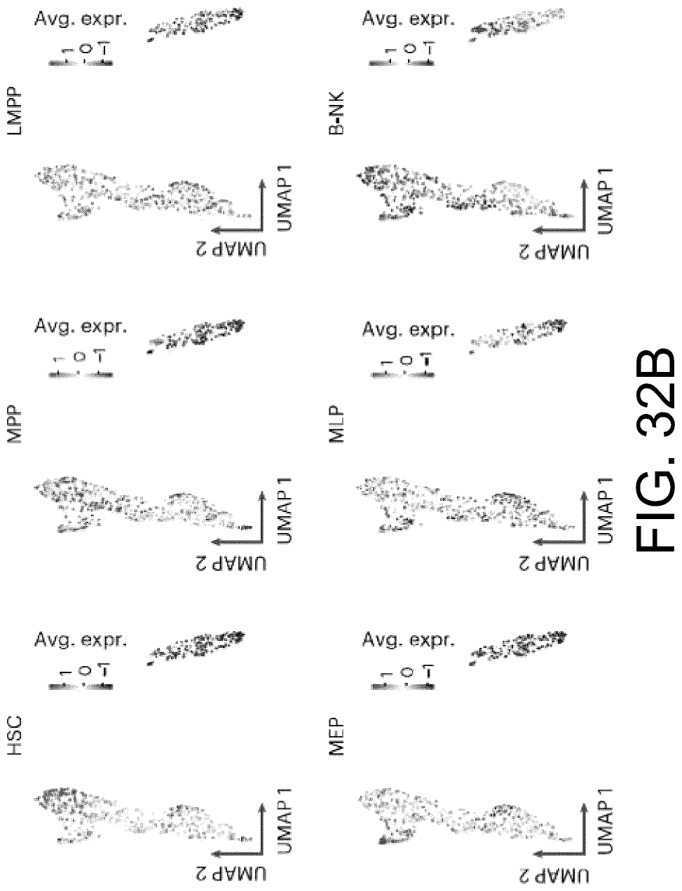

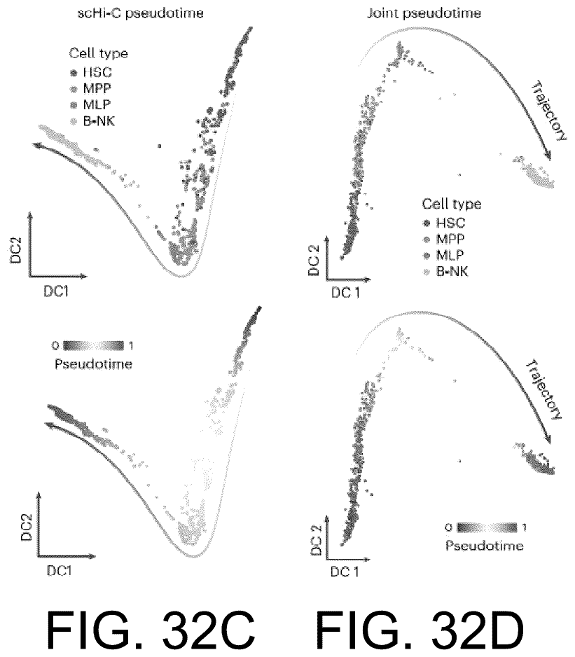

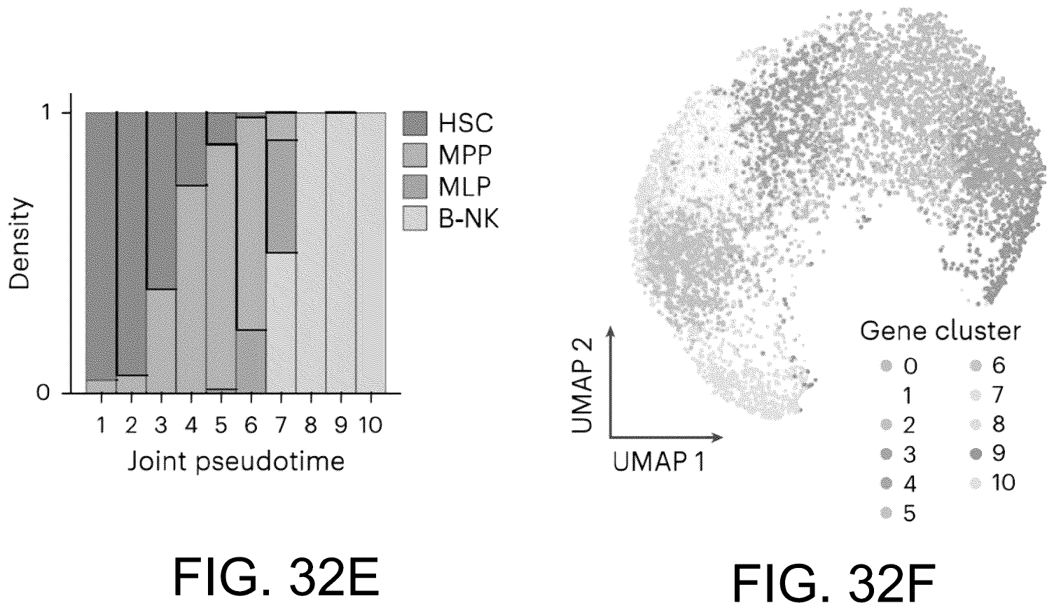

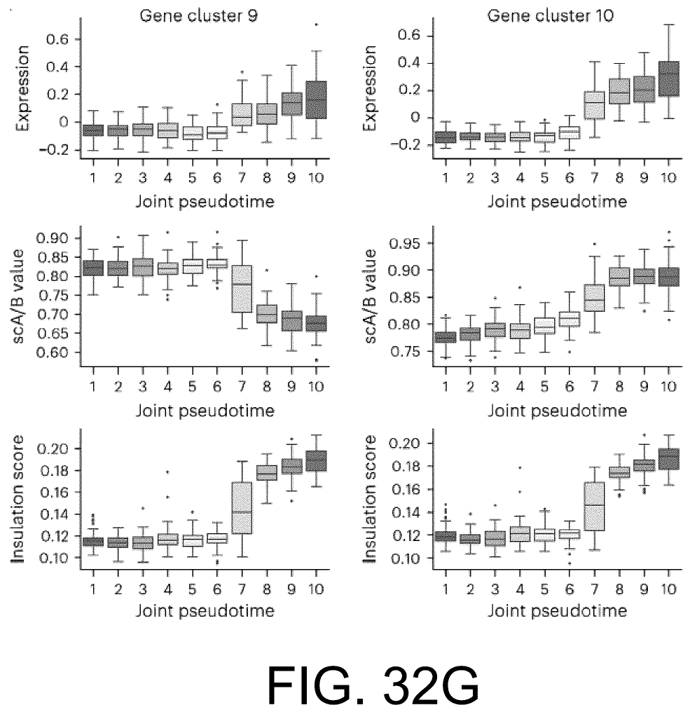

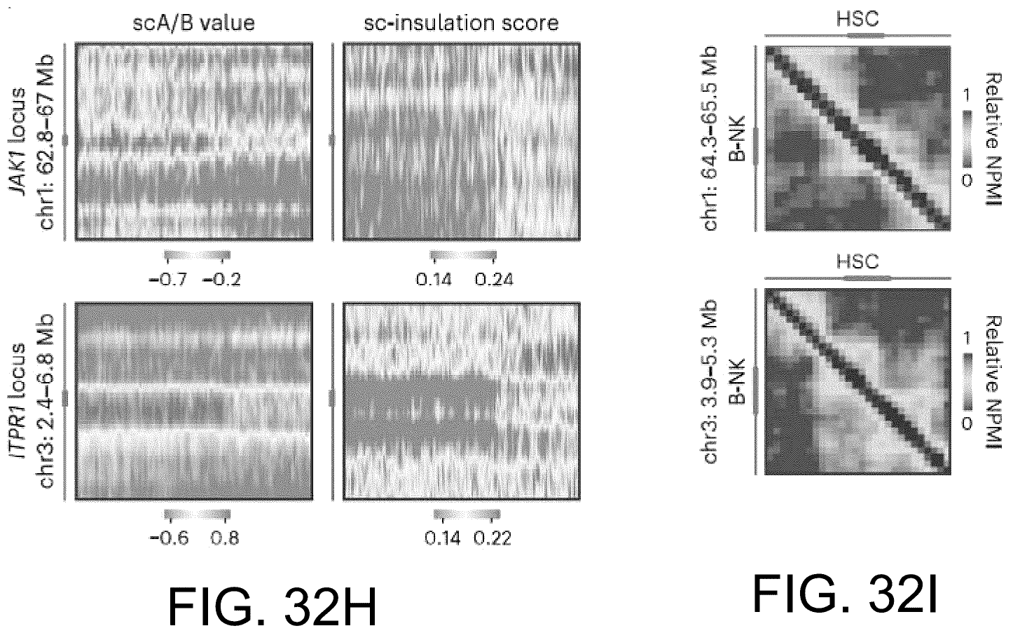

FIGS. 32A-321 illustrate the interplay between 3D genome variation and gene expression changes in human bone marrow differentiation. (32A) UMAP visualization of GAGE-seq scRNA-seq (left) and scHi-C profiles (right) of human bone marrow CD34+ cells. (32B) Average expression of known marker genes on the UMAP plot. The 6 panels include n=124, 78, 24, 82, 126, and 356 genes for HSC, MPP, LMPP, MEP, MLP, and B-NK, respectively. (32C, 32D) Inferred B-NK lineage trajectory and pseudotime from scHi-C profiles (32C) and jointly from scRNA-seq and scHi-C profiles (32D), displayed by cell type (upper) and pseudotime (lower). (32E) Cell type compositions across 10 equally divided pseudotime bins. (32F) UMAP visualization of gene clusters determined by the temporal trend of expression and scA/B value. (32G) Temporal trends of gene expression (upper row), scA/B value (middle row), and single-cell insulation score (lower row) of gene clusters 9 (left column) and 10 (right column). (32H) scA/B (left) and single-cell insulation score (right) of the JAK1 (upper) and ITPR1 (lower) loci (at 100-Kb resolution). Each row represents a cell, ordered by the joint pseudotime from left to right. Heat maps were smoothed by a Gaussian kernel with a receptive field of 10 neighboring cells and 1 neighboring bin in each direction. (32I) Pseudo-bulk contact maps (at 50-Kb resolution) of HSC and B—NK at the JAK1 (upper) and ITPR1 (lower) loci.

Unsupervised clustering of GAGE-seq scRNA-seq data revealed six clusters (five clusters with continuous diffusion and one distinct cluster), each displaying unique gene signatures (FIGS. 32A, 32B). Based on the gene expression signatures and known marker genes (Zhang Y, et al. Dev Cell 2022; 57:2745-2760), these clusters were annotated into known cell types: hematopoietic stem cell (HSC), multipotent progenitor (MPP), lymphoid-primed MPP (LMPP), multi-lymphoid progenitor (MLP), megakaryocyte-erythroid progenitor (MEP), and B lymphocyte natural killer cell progenitors (B-NK) (FIGS. 32A, 32B). These clusters, representing all three major blood cell lineages, showed a lymphoid lineage preference. The GAGE-seq scHi-C data also successfully resolved these six cell types (FIGS. 32A, 32B), further demonstrating the ability of the 3D genome to encode cell type information.

Focusing on four of the six identified cell types (HSC, MPP, MLP and B-NK), which represent early B-NK lineage, GAGE-seq was used to reconstruct the developmental trajectory, demonstrating the dynamic interplay between genome structure and gene expression along this trajectory. Transcriptome and 3D genome-based pseudotime trajectories, inferred from GAGE-seq data, were highly congruent (FIG. 32C), indicating that global 3D genome temporal variations overall mirror transcriptional changes and differentiation progression. Further, an integrated pseudotime trajectory was created (FIG. 32D), which was confirmed by the accurate alignment of the four cell types along the differentiation pseudotime and the observation that earlier-stage progenitors (e.g., HSCs) decrease while later-stage cells (e.g., B-NK) increase along the pseudotime (FIGS. 32D, 32E).

### Temporal Interplays Between 3D Genome and Gene Expression.

Comparisons between marker gene expression and 3D genome features in individual cell types during differentiation pseudotime suggest complex temporal interplay between both scA/B values and single-cell insulation scores with marker gene expressions.

An unsupervised clustering was then performed to further unravel relationships between gene expression and 3D genome features in the B-NK differentiation, based on all genes expressed in at least twenty single cells in the trajectory. 11 distinct gene clusters were identified (FIG. 32F). Notably, 5 of these 11 clusters showed a negative correlation between the changes in gene expression and scA/B value over pseudotime (FIG. 32G, left panel). Gene cluster 9 was closely examined, where expression increases while scA/B value decreases. Two genes were selected, JAK1 and ITPR1, which exhibit the highest similarity with the average temporal patterns of this gene cluster. Their scA/B value at the gene bodies indeed decreases over pseudotime without A/B compartment switches (FIG. 32H, left panels). This analysis identified gene groups with varied temporal patterns, including discordant patterns in expression and scA/B value, as reported previously (Tan L, Cell, supra), during differentiation.

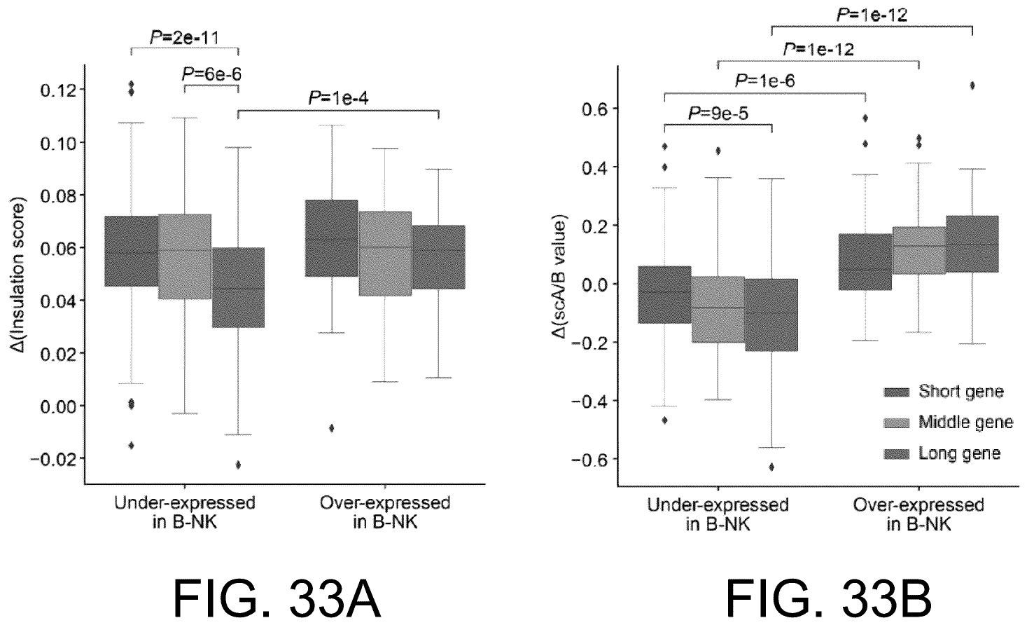

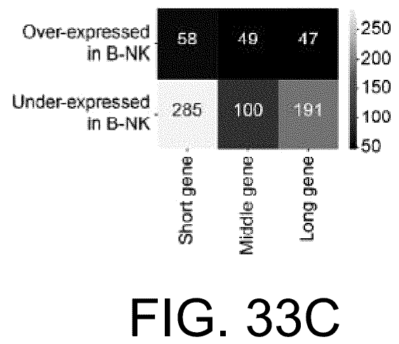

FIGS. 33A-33C illustrate the 3D genome reorganization at the gene loci of the B-NK cell differentially expressed (DE) genes with different gene lengths. (33A, 33B) Differential single-cell insulation score (33A) and scA/B value (33B) of B-NK DEGs. Genes are grouped by both gene length and differential expression. Short genes (length <100 Kb) are colored in blue. Middle genes (length in 100 Kb-200 Kb) are colored in orange. Long genes (length >200 Kb) are colored in green. (33C) The number of genes in each gene group. In (33A, 33B), P-values of one-sided t-test are shown. All boxplots show the median, first, and third quartiles, and whiskers extend no further than 1.5× interquartile range.

Regarding chromatin domains, a uniform temporal trend was observable in the aggregated single-cell insulation scores across all gene clusters, mirroring the pattern seen in the marker gene sets (FIG. 32G), indicating global 3D genome changes, manifested by widespread TAD-like domain re-organizations, in B-NK cells. For JAK1 and ITPR1, the single-cell insulation scores increased abruptly from MLP to B-NK, correlating with gene expression (FIG. 32H, right panels), supported by aggregated contact maps (FIG. 32I). Additionally, it was found that genes of different sizes appear to have distinct patterns with respect to single-cell insulation scores (FIGS. 33A-33C).

### DISCUSSION

The described high-throughput multiomic single-cell technology, GAGE-seq, delivers an integrative approach to co-assay 3D genome structure and gene expression in individual cells with high resolution. In this Example, it is demonstrated that GAGE-seq can reveal complex cell types from complex tissues not identified by other existing methods. Additionally, its data integration with spatial transcriptomic data points to great potential to reach a deeper understanding of 3D genome variation within complex tissues. Importantly, GAGE-seq also facilitates the reconstruction of differentiation trajectories based on 3D genome features, transcriptomes, or both. The disclosed integration of GAGE-seq with single-cell chromatin accessibility data further highlights the advantage of GAGE-seq in linking CREs and their target genes. The high congruence between these modalities underscores the intimate connection between the temporal variations of the 3D genome and transcriptional rewiring during cell differentiation. GAGE-seq has revealed much more nuanced relationships between 3D genome features and gene expression during bone marrow B-NK lineage differentiation, creating a resource for future studies to disentangle causal gene regulatory changes in differentiation through the lens of 3D genome in single cells.

GAGE-seq is characterized by its efficiency, scalability, robustness, cost-effectiveness, and adaptability. GAGE-seq, along with the described analytical tools, could significantly enhance the current toolkit for single-cell epigenomics. With wide-ranging applications, GAGE-seq can deepen the understanding of genome structure and function, providing insights into normal development and disease pathogenesis. GAGE-seq can be integrated with spatial labeling technologies, producing spatially-resolved scHi-C and scRNA-seq data. GAGE-seq offers the opportunity to integrate different molecular features in single cells, leading to a more comprehensive understanding of genome structure, cellular function, and their spatiotemporal variability.

### EXAMPLE CLAUSES

1. A method, including: receiving a single-cell suspension of permeabilized cells with permeabilized nuclei including RNA and cross-linked chromatin; generating transcriptomic DNA ((DNA) including a primer by reverse transcribing the RNA with the primer; generating first fragments by fragmenting the cross-linked chromatin using at least one first restriction enzyme (RE); generating ligated fragments by performing proximity ligation on the first fragments; generating spatial DNA (sDNA) by fragmenting the ligated fragments using at least one second RE; ligating first barcodes onto the tDNA and the sDNA; in response to ligating the first barcodes onto the tDNA and the sDNA, ligating second barcodes onto the tDNA and the sDNA; reverse-crosslinking the tDNA and the sDNA; removing cellular components from a solution including the tDNA and sDNA; and generating a tDNA library and an sDNA library by separating the tDNA and the sDNA.
2. The method of clause 1, wherein the primer includes a poly dT tail.
3. The method of clause 1 or 2, wherein the primer includes a biotinylated nucleotide.
4. The method of clause 3, wherein separating the tDNA and the sDNA includes: binding a magnetic bead to the biotinylated nucleotide; and capturing the tDNA by applying a magnetic field to the solution including the tDNA and sDNA.
5. The method of any of clauses 1-4, wherein the at least one first RE includes Msel and/or CviQI.
6. The method of any of clauses 1-5, wherein the at least one first RE includes a 4-cut RE.
7. The method of any of clauses 1-6, wherein the first fragments include a 5′-TA.
8. The method of any of clauses 1-7, wherein generating the first fragments is performed at a temperature in a range of about 20 degrees Celsius (20° C.) to about 30° C.
9. The method of any of clauses 1-8, wherein generating the ligated fragments is performed at a temperature in a range of about 10° C. to about 20° C.
10. The method of any of clauses 1-9, wherein the at least one second RE includes Ddel.
11. The method of any of clauses 1-10, wherein generating the sDNA includes is performed at a temperature in a range of about 30° C. to about 40° C.
12. The method of any of clauses 1-11, wherein generating the tDNA, generating the first fragments, generating the ligated fragments, generating the sDNA, and ligating the first barcodes onto the tDNA and the sDNA are performed in the presence of the permeabilized cells and/or intact nuclei of the permeabilized cells.
13. The method of any of clauses 1-12, further including: sequencing the tDNA library; and sequencing the sDNA library.
14. The method of clause 13, wherein sequencing the tDNA library includes performing at least one of a massively parallel sequencing (MPS) technique, next generation sequencing, targeted sequencing, direct sequencing, Sanger sequencing, sequencing-by-synthesis, or nanopore sequencing on the tDNA library, and wherein sequencing the sDNA library includes performing at least one of a massively parallel sequencing (MPS) technique, next generation sequencing, targeted sequencing, direct sequencing, Sanger sequencing, sequencing-by-synthesis, or nanopore sequencing on the sDNA library.
15. The method of any of clauses 1-14, further including: in response to ligating the second barcodes onto the tDNA and the sDNA, ligating third barcodes onto the tDNA and the sDNA.
16. A system, including: a fluidic circuit configured to receive a suspension of permeabilized cells including RNA and cross-linked chromatin; at least one storage receptacle configured to store a first reagent, a second reagent, a third reagent, a fourth reagent, a fifth reagent, a sixth reagent, and a seventh reagent; at least one pump configured to move the at least one reagent through the fluidic circuit; and at least one processor configured to: cause generation of transcriptomic DNA ((DNA) including a primer by causing the at least one pump to move the first reagent into the fluidic circuit, the first reagent including the primer and reverse transcriptase; cause generation of first fragments by causing the at least one pump to move the second reagent into the fluidic circuit, the second reagent including at least one first restriction enzyme (RE) that fragments the cross-linked chromatin; cause generation of ligated fragments by causing the at least one pump to move the third reagent into the fluidic circuit, the third reagent including one or more components that perform proximity ligation on the first fragments; cause generation of spatial DNA (sDNA) by causing the at least one pump to move the fourth reagent into the fluidic circuit, the fourth reagent including at least one second RE that fragments the ligated fragments; cause ligation of first barcodes onto the tDNA and the sDNA by causing the at least one pump to move the fifth reagent into the fluidic circuit; in cause ligation of second barcodes onto the tDNA and the sDNA by causing the at least one pump to move the sixth reagent into the fluidic circuit; cause reverse-crosslinking of the tDNA and the sDNA by causing the at least one pump to move the seventh reagent into the fluidic circuit; and cause generation of a tDNA library and an sDNA library by causing the at least one pump to separate, in the fluidic circuit, a solution including the tDNA from a solution including the sDNA.
17. The system of clause 16, further including: a heater configured to maintain a temperature of the fluidic circuit in a range of about 10° C. to about 40° C.
18. The system of clause 16 or 17, further including: a centrifuge configured to centrifuge at least one portion of the fluidic circuit.
19. The system of any of clauses 16-18, further including: a sequencer configured to generate sequence read data by sequencing the tDNA library and the sDNA library.
20. The system of any of clauses 16-20, wherein the processor is further configured to: cause removal of cellular components from a solution including the tDNA and sDNA by causing the at least one pump to move a waste solution out of the fluidic circuit, the waste solution including the cellular components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

This disclosure refers to several references, including articles, books, references, conferences, and other publications. Each one of these references is incorporated by reference herein in its entirety.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
receiving a single-cell suspension of permeabilized cells with permeabilized nuclei comprising RNA and cross-linked chromatin;
generating transcriptomic DNA ((DNA) comprising a primer by reverse transcribing the RNA with the primer;
generating first fragments by fragmenting the cross-linked chromatin using at least one first restriction enzyme (RE);
generating ligated fragments by performing proximity ligation on the first fragments;
generating spatial DNA (sDNA) by fragmenting the ligated fragments using at least one second RE;
ligating first barcodes onto the tDNA and the sDNA;
in response to ligating the first barcodes onto the tDNA and the sDNA, ligating second barcodes onto the tDNA and the sDNA;
reverse-crosslinking the tDNA and the sDNA;
removing cellular components from a solution comprising the tDNA and sDNA; and
generating a tDNA library and an sDNA library by separating the tDNA and the sDNA.

2. The method of claim 1, wherein the primer comprises a poly dT tail.

3. The method of claim 1, wherein the primer comprises a biotinylated nucleotide.

4. The method of claim 3, wherein separating the tDNA and the sDNA comprises:
binding a magnetic bead to the biotinylated nucleotide; and
capturing the tDNA by applying a magnetic field to the solution comprising the tDNA and sDNA.

5. The method of claim 1, wherein the at least one first RE comprises Msel and/or CviQI.

6. The method of claim 1, wherein the at least one first RE comprises a 4-cut RE.

7. The method of claim 1, wherein the first fragments comprise a 5′-TA.

8. The method of claim 1, wherein generating the first fragments is performed at a temperature in a range of about 20 degrees Celsius (20° C.) to about 30° C.

9. The method of claim 1, wherein generating the ligated fragments is performed at a temperature in a range of about 10° C. to about 20° C.

10. The method of claim 1, wherein the at least one second RE comprises Ddel.

11. The method of claim 1, wherein generating the sDNA comprises is performed at a temperature in a range of about 30° C. to about 40° C.

12. The method of claim 1, wherein generating the tDNA, generating the first fragments, generating the ligated fragments, generating the sDNA, and ligating the first barcodes onto the tDNA and the sDNA are performed in the presence of the permeabilized cells and/or intact nuclei of the permeabilized cells.

13. The method of claim 1, further comprising:
sequencing the tDNA library; and
sequencing the sDNA library.

14. The method of claim 13, wherein sequencing the tDNA library comprises performing at least one of a massively parallel sequencing (MPS) technique, next generation sequencing, targeted sequencing, direct sequencing, Sanger sequencing, sequencing-by-synthesis, or nanopore sequencing on the tDNA library, and
wherein sequencing the sDNA library comprises performing at least one of a massively parallel sequencing (MPS) technique, next generation sequencing, targeted sequencing, direct sequencing, Sanger sequencing, sequencing-by-synthesis, or nanopore sequencing on the sDNA library.

15. The method of claim 1, further comprising:
in response to ligating the second barcodes onto the tDNA and the sDNA, ligating third barcodes onto the tDNA and the sDNA.

16. A system, comprising:
a fluidic circuit configured to receive a suspension of permeabilized cells comprising RNA and cross-linked chromatin;
at least one storage receptacle configured to store a first reagent, a second reagent, a third reagent, a fourth reagent, a fifth reagent, a sixth reagent, and a seventh reagent;
at least one pump configured to move the at least one reagent through the fluidic circuit; and
at least one processor configured to:
cause generation of transcriptomic DNA ((DNA) comprising a primer by causing the at least one pump to move the first reagent into the fluidic circuit, the first reagent comprising the primer and reverse transcriptase;
cause generation of first fragments by causing the at least one pump to move the second reagent into the fluidic circuit, the second reagent comprising at least one first restriction enzyme (RE) that fragments the cross-linked chromatin;
cause generation of ligated fragments by causing the at least one pump to move the third reagent into the fluidic circuit, the third reagent comprising one or more components that perform proximity ligation on the first fragments;
cause generation of spatial DNA (sDNA) by causing the at least one pump to move the fourth reagent into the fluidic circuit, the fourth reagent comprising at least one second RE that fragments the ligated fragments;
cause ligation of first barcodes onto the tDNA and the sDNA by causing the at least one pump to move the fifth reagent into the fluidic circuit;
in cause ligation of second barcodes onto the tDNA and the sDNA by causing the at least one pump to move the sixth reagent into the fluidic circuit;
cause reverse-crosslinking of the tDNA and the sDNA by causing the at least one pump to move the seventh reagent into the fluidic circuit; and
cause generation of a tDNA library and an sDNA library by causing the at least one pump to separate, in the fluidic circuit, a solution comprising the tDNA from a solution comprising the sDNA.

17. The system of claim 16, further comprising:
a heater configured to maintain a temperature of the fluidic circuit in a range of about 10° C. to about 40° C.

18. The system of claim 16, further comprising:
a centrifuge configured to centrifuge at least one portion of the fluidic circuit.

19. The system of claim 16, further comprising:
a sequencer configured to generate sequence read data by sequencing the tDNA library and the sDNA library.

20. The system of claim 16, wherein the processor is further configured to:
cause removal of cellular components from a solution comprising the tDNA and sDNA by causing the at least one pump to move a waste solution out of the fluidic circuit, the waste solution comprising the cellular components.


SEQUENCE LISTING (2 sequences)

SEQ ID NO: 1 - DNA, 16 bp, synthetic construct
  source 1..16: mol_type = other DNA
cacgccacatgtcaat

SEQ ID NO: 2 - DNA, 23 bp, synthetic construct
  source 1..23: mol_type = other DNA
ggtgggaattaatttgtacaaga